SEQUENCE LISTING (30 sequences)

SEQ ID NO: 1 - protein, 1031 aa, synthetic construct
  REGION 1..1031: note = Synthetic
INGQQYYIDPTTGQPRKNFLLQNGNDWIYFDKDTGAGTNALKLQFDKGTISADEQYRRGNEAYSYDDKSIENVNGYLTADTWYRPKQILKDGTTWTDSKETDMRPILMVWWPNTVTQAYYLNYMKQYGNLLPASLPSFSTDADSAELNHYSELVQQNIEKRISETGSTDWLRTLMHEFVTKNSMWNKDSENVDYGGLQLQGGFLKYVNSDLTKYANSDWRLMNRTATNIDGKNYGGAEFLLANDIDNSNPVVQAEELNWLYYLMNFGTITGNNPEANFDGIRVDAVDNVDVDLLSIARDYFNAAYNMEQSDASANKHINILEDWGWDDPAYVNKIGNPQLTMDDRLRNAIMDTLSGAPDKNQALNKLITQSLVNRANDNTENAVIPSYNFVRAHDSNAEDQIRQAIQAATGKPYGEFNLDDEKKGMEAYINDQNSTNKKWNLYNMPSAYTILLTNKDSVPRVYYGDLYQDGGQYMEHKTRYFDTITNLLKTRVKYVAGGQTMSVDKNGILTNVRFGKGAMNATDTGTDETRTEGIGVVISNNTNLKLNDGESVVLHMGAAHKNQKYRAVILTTEDGVKNYTNDTDAPVAYTDANGDLHFTNTNLDGQQYTAVRGYANPDVTGYLAVWVPAGAADDQDARTAPSDEAHTTKTAYRSNAALDSNVIYEGFSNFIYWPTTESERTNVRIAQNADLFKSWGITTFELAPQYNSSKDGTFLDSIIDNGYAFTDRYDLGMSTPNKYGSDEDLRNALQALHKAGLQAIADWVPDQIYNLPGKEAVTVTRSDDHGTTWEVSPIKNVVYITNTIGGGEYQKKYGGEFLDTLQKEYPQLFSQVYPVTQTTIDPSVKIKEWSAKYFNGTNILHRGAGYVLRSNDGKYYNLGTSTQQFLPSQLSVQDNEGYGFVKEGNNYHYYDENKQMVKDAFIQDSVGNWYYFDKNGNMVANQSPVEISSNGASGTYLFLNNGTSFRSGLVKTDAGTYYYDGDGRMVRNQTVSDGAMTYVLDENGKLVSESFDSSATEAHPLKPGDLNGQK

SEQ ID NO: 2 - DNA, 3099 bp, synthetic construct
  misc_feature 1..3099: note = Synthetic
  source 1..3099: mol_type = other DNA
atgatcaatggccagcagtattatatcgatccgacgaccggtcagcctcgcaaaaactttttgctgcaaaatgggaacgactggatttactttgacaaagataccggggccggtacgaatgccctcaaactgcaatttgacaagggcactatcagcgcggacgaacagtaccgacgtggtaatgaagcgtacagctatgatgataaatctattgaaaatgtcaatgggtatctgacagcagatacttggtatcgccccaagcagattctgaaagacggcaccacatggacggatagcaaagaaactgacatgcgcccgatcttaatggtctggtggcccaataccgtgacgcaggcctactatctgaattacatgaaacagtatggaaaccttctgccggcctctctgccatcttttagcaccgatgctgattccgctgaattaaaccattatagcgagctagttcaacagaacatcgagaaaagaattagtgaaacgggaagcactgactggctgcggaccttgatgcatgaatttgtgacgaaaaacagcatgtggaacaaagattctgaaaatgtagattatggtggcctccaacttcaggggggtttcctgaaatacgtgaactccgatttaactaagtacgccaatagcgattggagactgatgaaccgcaccgcgacgaacattgatggcaaaaattacggaggtgccgaatttcttttggccaacgacattgacaatagcaacccagtagttcaagcagaggaactgaactggctgtattacctcatgaactttgggaccattaccggcaataatcctgaagctaattttgatgggattcgagttgatgcggtggataatgtggacgttgatcttctgtctattgctcgcgactactttaatgcagcatacaacatggaacaatcagatgcttcggccaacaaacatatcaacattctcgaagattggggctgggatgacccagcgtacgttaacaagattggcaaccctcagttaacgatggacgaccgcctgcgtaacgcgattatggatacgctgtcaggtgcgccagacaaaaatcaggcgctgaacaagctgatcacacaatcgctggtgaacagagcgaacgataataccgaaaacgcggttattccatcctacaattttgtccgcgctcacgatagtaatgccgaagatcaaattcgacaggcaatccaggcggccacaggtaaaccgtacggagagtttaatctcgacgacgaaaaaaaaggtatggaagcgtacattaacgaccaaaattcgaccaacaagaaatggaacctgtacaacatgccttccgcgtacacgattcttctgaccaacaaagatagcgtaccgagggtgtattacggggatttataccaggatggcggccagtacatggagcacaagacccgctattttgacaccatcacaaacctgctgaaaacccgtgtaaaatatgtagctggcggtcagaccatgagtgttgataaaaatggcattctgaccaatgttcgttttggtaaaggagcgatgaatgctacggataccggcaccgacgagacacgtacggagggcattggtgttgtaatatcgaacaataccaacttgaagcttaacgatggagaaagcgtggtactgcacatgggtgcggcgcataaaaatcaaaaataccgtgcggtgatcttgacgacggaagatggagtaaaaaattatacgaacgatacagacgcacccgtggcctacaccgatgctaacggcgatctgcatttcacgaataccaatctggatggccagcaatatactgcggtacgcggttatgctaacccggatgtgaccggctaccttgctgtttgggtgccggcaggggcggcggatgatcaggatgcccgcactgctccgagcgatgaggcgcatacgacaaaaactgcgtatagatcgaatgcagccttggattctaacgttatatacgaaggttttagcaactttatttattggccgactaccgaaagcgaacgaacaaacgtacgcattgctcaaaatgcggacctgtttaaaagctggggaatcaccacctttgaactggccccgcaatacaattcatcaaaagacggcacctttctggattcaattattgataacggctatgcatttaccgatcgttacgatctggggatgtctaccccgaacaaatacggctctgatgaggatctccgcaatgcgctacaagccttacacaaggccggactccaagcgattgctgactgggtgccggatcagatttataatctgccaggtaaggaagctgtgaccgtaacccgtagcgatgaccacggcaccacgtgggaagtctcaccgatcaagaacgttgtctacatcacgaataccattggtggcggtgaataccagaagaaatatggtggcgaatttttggacaccttgcaaaaagaatacccgcagctgttctcacaggtatacccagtgacccaaaccaccatcgatccgagcgtcaaaataaaagaatggagtgcaaagtattttaacggcacaaatattcttcatcggggcgcagggtacgtactccgtagcaatgacggcaaatactataatctcggaacctctactcagcagttcttgccgagccagctgtcagttcaagataacgagggatatgggtttgtcaaagaaggtaacaattatcattactatgatgagaataaacaaatggtaaaggacgcatttatccaggactctgttggtaattggtattactttgacaagaacggtaacatggtggcaaaccagtccccggtggaaatcagttcaaatggggcgtcgggcacgtatctcttcctgaataacggcacctccttccgtagtggcttagttaagaccgatgcaggaacctactactatgatggggacggtcgtatggtgcggaatcaaacggtttctgacggtgccatgacctacgtgttggacgaaaacggaaaactcgtgtccgaatcttttgatagttcggcgaccgaagcccacccacttaagccaggcgacctcaacggccagaagtaa

SEQ ID NO: 3 - protein, 462 aa, synthetic construct
  REGION 1..462: note = Synthetic
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 4 - DNA, 1389 bp, synthetic construct
  misc_feature 1..1389: note = Synthetic
  source 1..1389: mol_type = other DNA
atggattcgggttactcttcctcctatgcggcggctgcgggtatgcacgttgttatctgtccgtggctggcttttggtcacctgctgccgtgcctggatctggcacagcgtctggcttcacgcggccatcgtgtcagcttcgtgtctaccccgcgcaatatttcgcgtctgccgccggttcgtccggcactggctccgctggttgcatttgtcgctctgccgctgccgcgcgtggaaggtctgccggatggtgcggaaagtaccaacgacgtgccgcatgatcgcccggacatggttgaactgcaccgtcgtgcattcgatggtctggcagcaccgttttccgaatttctgggtacggcgtgcgccgattgggtgatcgttgacgtctttcatcactgggcggcggcggcggcgctggaacataaagttccgtgtgcaatgatgctgctgggctcagctcacatgattgcgtcgatcgcagaccgtcgcctggaacgtgcagaaaccgaaagtccggctgcggccggccagggtcgcccggcagctgcgccgaccttcgaagtggcccgcatgaaactgattcgtacgaaaggcagctctggtatgagcctggcagaacgctttagtctgaccctgtcccgtagttccctggtggttggtcgcagttgcgttgaatttgaaccggaaaccgtcccgctgctgtccacgctgcgtggtaaaccgatcacctttctgggtctgatgccgccgctgcatgaaggccgtcgcgaagatggtgaagacgcaacggtgcgttggctggatgcacagccggctaaaagcgtcgtgtatgtcgccctgggctctgaagtgccgctgggtgtggaaaaagttcacgaactggcactgggcctggaactggctggcacccgcttcctgtgggcactgcgtaaaccgacgggtgtgagcgatgcggacctgctgccggccggttttgaagaacgtacccgcggccgtggtgttgtcgcaacgcgttgggtcccgcaaatgagcattctggcgcatgccgcagtgggcgcctttctgacccactgtggttggaacagcacgatcgaaggcctgatgtttggtcacccgctgattatgctgccgatcttcggcgatcagggtccgaacgcacgtctgattgaagcgaaaaatgccggcctgcaagttgcgcgcaacgatggcgacggttctttcgaccgtgagggtgtggctgcggccattcgcgcagtggctgttgaagaagaatcatcgaaagtttttcaggcgaaagccaaaaaactgcaagaaatcgtcgcggatatggcctgccacgaacgctacattgatggtttcattcagcaactgcgctcctacaaagactaa

SEQ ID NO: 5 - protein, 462 aa, synthetic construct
  REGION 1..462: note = Synthetic
MGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTK

SEQ ID NO: 6 - DNA, 1389 bp, synthetic construct
  misc_feature 1..1389: note = Synthetic
  source 1..1389: mol_type = other DNA
atgggtagctcgggcatgtccctggcggaacgcttttcgctgacgctgagtcgctcatccctggttgttggtcgcagttgtgttgaatttgaaccggaaaccgttccgctgctgtctacgctgcgcggcaaaccgattaccttcctgggtctgatgccgccgctgcatgaaggccgtcgcgaagatggtgaagacgccacggtgcgttggctggatgctcagccggcgaaatcggtggtttatgtcgcactgggcagcgaagtgccgctgggtgtcgaaaaagtgcacgaactggccctgggcctggaactggcaggcacccgctttctgtgggcactgcgtaaaccgacgggcgttagcgatgctgacctgctgccggcgggtttcgaagaacgcacccgcggccgtggtgtcgtggccacccgttgggtgccgcaaatgtccattctggctcatgcggccgttggcgcatttctgacccactgcggttggaacagcacgatcgaaggcctgatgtttggtcatccgctgattatgctgccgatcttcggcgatcagggtccgaacgcacgcctgatcgaagccaaaaatgcaggcctgcaagttgcgcgtaacgatggcgacggtagctttgaccgcgaaggtgtcgcagctgcgattcgtgctgtggcggttgaagaagaaagcagcaaagtcttccaggccaaagcgaaaaaactgcaagaaatcgtggctgatatggcgtgtcatgaacgctatattgacggctttatccagcaactgcgttcttacaaagatgacagtggctatagttcctcatacgccgcagctgcgggtatgcatgttgtcatttgcccgtggctggcgtttggtcacctgctgccgtgtctggatctggcacagcgcctggcatctcgcggtcaccgtgtttcgttcgtcagcaccccgcgcaatatcagtcgtctgccgccggttcgtccggcgctggcgccgctggttgcgttcgttgcactgccgctgccgcgtgtggaaggtctgccggatggtgccgaatcgaccaacgacgttccgcatgatcgtccggacatggtcgaactgcatcgtcgcgcctttgatggcctggccgcaccgtttagcgaatttctgggtacggcctgcgcagattgggtcattgtggacgtttttcaccactgggcggcggcggcggcgctggaacataaagtgccgtgtgcgatgatgctgctgggttccgcccacatgattgcttcaatcgcggatcgtcgcctggaacgtgccgaaaccgaaagtccggcggcggcaggccagggtcgtccggcggcggcaccgacctttgaagtggcacgtatgaaactgattcgcacgaaataa

SEQ ID NO: 7 - protein, 459 aa, synthetic construct
  REGION 1..459: note = Synthetic
MDGNSSSSPLHVVICPWLALGHLLPCLDIAERLASRGHRVSFVSTPRNIARLPPLRPAVAPLVDFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKA

SEQ ID NO: 8 - DNA, 1380 bp, synthetic construct
  misc_feature 1..1380: note = Synthetic
  source 1..1380: mol_type = other DNA
atggatggtaactcctcctcctcgccgctgcatgtggtcatttgtccgtggctggctctgggtcacctgctgccgtgtctggatattgctgaacgtctggcgtcacgcggccatcgtgtcagttttgtgtccaccccgcgcaacattgcccgtctgccgccgctgcgtccggctgttgcaccgctggttgatttcgtcgcactgccgctgccgcatgttgacggtctgccggagggtgcggaatcgaccaatgatgtgccgtatgacaaatttgaactgcaccgtaaggcgttcgatggtctggcggccccgtttagcgaatttctgcgtgcagcttgcgcagaaggtgcaggttctcgcccggattggctgattgtggacacctttcatcactgggcggcggcggcggcggtggaaaacaaagtgccgtgtgttatgctgctgctgggtgcagcaacggtgatcgctggtttcgcgcgtggtgttagcgaacatgcggcggcggcggtgggtaaagaacgtccggctgcggaagccccgagttttgaaaccgaacgtcgcaagctgatgaccacgcagaatgcctccggcatgaccgtggcagaacgctatttcctgacgctgatgcgtagcgatctggttgccatccgctcttgcgcagaatgggaaccggaaagcgtggcagcactgaccacgctggcaggtaaaccggtggttccgctgggtctgctgccgccgagtccggaaggcggtcgtggcgtttccaaagaagatgctgcggtccgttggctggacgcacagccggcaaagtcagtcgtgtacgtcgcactgggttcggaagtgccgctgcgtgcggaacaagttcacgaactggcactgggcctggaactgagcggtgctcgctttctgtgggcgctgcgtaaaccgaccgatgcaccggacgccgcagtgctgccgccgggtttcgaagaacgtacccgcggccgtggtctggttgtcacgggttgggtgccgcagattggcgttctggctcatggtgcggtggctgcgtttctgacccactgtggctggaactctacgatcgaaggcctgctgttcggtcatccgctgattatgctgccgatcagctctgatcagggtccgaatgcgcgcctgatggaaggccgtaaagtcggtatgcaagtgccgcgtgatgaatcagacggctcgtttcgtcgcgaagatgttgccgcaaccgtccgcgccgtggcagttgaagaagacggtcgtcgcgtcttcacggctaacgcgaaaaagatgcaagaaattgtggccgatggcgcatgccacgaacgttgtattgacggttttatccagcaactgcgcagttacaaggcgtaa

SEQ ID NO: 9 - protein, 458 aa, synthetic construct
  REGION 1..458: note = Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 10 - DNA, 1377 bp, synthetic construct
  misc_feature 1..1377: note = Synthetic
  source 1..1377: mol_type = other DNA
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattctgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttataa

SEQ ID NO: 11 - protein, 458 aa, synthetic construct
  REGION 1..458: note = Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 12 - DNA, 1377 bp, synthetic construct
  misc_feature 1..1377: note = Synthetic
  source 1..1377: mol_type = other DNA
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattgcgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttataa

SEQ ID NO: 13 - protein, 458 aa, synthetic construct
  REGION 1..458: note = Synthetic
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 14 - DNA, 1377 bp, synthetic construct
  misc_feature 1..1377: note = Synthetic
  source 1..1377: mol_type = other DNA
atgaactggcaaatcctgaaagaaatcctgggtaaaatgatcaaacaaaccaaagcgtcgtcgggcgttatctggaactccttcaaagaactggaagaatcagaactggaaaccgttattcgcgaaatcccggctccgtcgttcctgattccgctgccgaaacatctgaccgcgagcagcagcagcctgctggatcacgaccgtacggtctttcagtggctggatcagcaaccgccgtcatcggtgctgtatgtttcattcggtagcacctctgaagtcgatgaaaaagactttctggaaatcgctcgcggcctggtggatagtaaacagtccttcctgtgggtggttcgtccgggttttgtgaaaggcagcacgtgggttgaaccgctgccggatggcttcctgggtgaacgcggccgtattgtcaaatgggtgccgcagcaagaagtgctggcacatggtgctatcggcgcgttttggacccactctggttggaacagtacgctggaatccgtttgcgaaggtgtcccgatgattttcagcgattttggcctggaccagccgctgaatgcccgctatatgtctgatgttctgaaagtcggtgtgtacctggaaaacggttgggaacgtggcgaaattgcgaatgccatccgtcgcgttatggtcgatgaagaaggcgaatacattcgccagaacgctcgtgtcctgaaacaaaaagcggacgtgagcctgatgaaaggcggtagctcttatgaatcactggaatcgctggttagctacatcagttccctggaaaataaaaccgaaaccacggtgcgtcgccgtcgccgtattatcctgttcccggttccgtttcagggtcatattaacccgatcctgcaactggcgaatgttctgtattcaaaaggcttttcgatcaccatcttccatacgaacttcaacaaaccgaaaaccagtaactacccgcactttacgttccgctttattctggataacgacccgcaggatgaacgtatctccaatctgccgacccacggcccgctggccggtatgcgcattccgattatcaatgaacacggtgcagatgaactgcgccgtgaactggaactgctgatgctggccagtgaagaagatgaagaagtgtcctgtctgatcaccgacgcactgtggtatttcgcccagagcgttgcagattctctgaacctgcgccgtctggtcctgatgacgtcatcgctgttcaattttcatgcgcacgtttctctgccgcaatttgatgaactgggctacctggacccggatgacaaaacccgtctggaagaacaagccagtggttttccgatgctgaaagtcaaagacattaaatccgcctattcgtaa

SEQ ID NO: 15 - protein, 1031 aa, synthetic construct
  REGION 1..1031: note = Synthetic
INGQQYYIDPTTGQPRKNFLLQNGNDWIYFDKDTGAGTNALKLQFDKGTISADEQYRRGNEAYSYDDKSIENVNGYLTADTWYRPKQILKDGTTWTDSKETDMRPILMVWWPNTVTQAYYLNYMKQYGNLLPASLPSFSTDADSAELNHYSELVQQNIEKRISETGSTDWLRTLMHEFVTKNSMWNKDSENVDYGGLQLQGGFLKYVNSDLTKYANSDWRLMNRTATNIDGKNYGGAEFLLANDIDNSNPVVQAEELNWLYYLMNFGTITGNNPEANFDGIRVDAVDNVDVDLLSIARDYFNAAYNMEQSDASANKHINILEDWGWDDPAYVNKIGNPQLTMDDRLRNAIMDTLSGAPDKNQALNKLITQSLVNRANDNTENAVIPSYNFVRAHDSNAQDQIRQAIQAATGKPYGEFNLDDEKKGMEAYINDQNSTNKKWNLYNMPSAYTILLTNKDSVPRVYYGDLYQDGGQYMEHKTRYFDTITNLLKTRVKYVAGGQTMSVDKNGILTNVRFGKGAMNATDTGTDETRTEGIGVVISNNTNLKLNDGESVVLHMGAAHKNQKYRAVILTTEDGVKNYTNDTDAPVAYTDANGDLHFTNTNLDGQQYTAVRGYANPDVTGYLAVWVPAGAADDQDARTAPSDEAHTTKTAYRSNAALDSNVIYEGFSNFIYWPTTESERTNVRIAQNADLFKSWGITTFELAPQYNSSKDGTFLDSIIDNGYAFTDRYDLGMSTPNKYGSDEDLRNALQALHKAGLQAIADWVPDQIYNLPGKEAVTVTRSDDHGTTWEVSPIKNVVYITNTIGGGEYQKKYGGEFLDTLQKEYPQLFSQVYPVTQTTIDPSVKIKEWSAKYFNGTNILHRGAGYVLRSNDGKYYNLGTSTQQFLPSQLSVQDNEGYGFVKEGNNYHYYDENKQMVKDAFIQDSVGNWYYFDKNGNMVANQSPVEISSNGASGTYLFLNNGTSFRSGLVKTDAGTYYYDGDGRMVRNQTVSDGAMTYVLDENGKLVSESFDSSATEAHPLKPGDLNGQK

SEQ ID NO: 16 - DNA, 3096 bp, synthetic construct
  misc_feature 1..3096: note = Synthetic
  source 1..3096: mol_type = other DNA
atcaatggccagcagtattatatcgatccgacgaccggtcagcctcgcaaaaactttttgctgcaaaatgggaacgactggatttactttgacaaagataccggggccggtacgaatgccctcaaactgcaatttgacaagggcactatcagcgcggacgaacagtaccgacgtggtaatgaagcgtacagctatgatgataaatctattgaaaatgtcaatgggtatctgacagcagatacttggtatcgccccaagcagattctgaaagacggcaccacatggacggatagcaaagaaactgacatgcgcccgatcttaatggtctggtggcccaataccgtgacgcaggcctactatctgaattacatgaaacagtatggaaaccttctgccggcctctctgccatcttttagcaccgatgctgattccgctgaattaaaccattatagcgagctagttcaacagaacatcgagaaaagaattagtgaaacgggaagcactgactggctgcggaccttgatgcatgaatttgtgacgaaaaacagcatgtggaacaaagattctgaaaatgtagattatggtggcctccaacttcaggggggtttcctgaaatacgtgaactccgatttaactaagtacgccaatagcgattggagactgatgaaccgcaccgcgacgaacattgatggcaaaaattacggaggtgccgaatttcttttggccaacgacattgacaatagcaacccagtagttcaagcagaggaactgaactggctgtattacctcatgaactttgggaccattaccggcaataatcctgaagctaattttgatgggattcgagttgatgcggtggataatgtggacgttgatcttctgtctattgctcgcgactactttaatgcagcatacaacatggaacaatcagatgcttcggccaacaaacatatcaacattctcgaagattggggctgggatgacccagcgtacgttaacaagattggcaaccctcagttaacgatggacgaccgcctgcgtaacgcgattatggatacgctgtcaggtgcgccagacaaaaatcaggcgctgaacaagctgatcacacaatcgctggtgaacagagcgaacgataataccgaaaacgcggttattccatcctacaattttgtccgcgctcacgatagtaatgcccaggatcaaattcgacaggcaatccaggcggccacaggtaaaccgtacggagagtttaatctcgacgacgaaaaaaaaggtatggaagcgtacattaacgaccaaaattcgaccaacaagaaatggaacctgtacaacatgccttccgcgtacacgattcttctgaccaacaaagatagcgtaccgagggtgtattacggggatttataccaggatggcggccagtacatggagcacaagacccgctattttgacaccatcacaaacctgctgaaaacccgtgtaaaatatgtagctggcggtcagaccatgagtgttgataaaaatggcattctgaccaatgttcgttttggtaaaggagcgatgaatgctacggataccggcaccgacgagacacgtacggagggcattggtgttgtaatatcgaacaataccaacttgaagcttaacgatggagaaagcgtggtactgcacatgggtgcggcgcataaaaatcaaaaataccgtgcggtgatcttgacgacggaagatggagtaaaaaattatacgaacgatacagacgcacccgtggcctacaccgatgctaacggcgatctgcatttcacgaataccaatctggatggccagcaatatactgcggtacgcggttatgctaacccggatgtgaccggctaccttgctgtttgggtgccggcaggggcggcggatgatcaggatgcccgcactgctccgagcgatgaggcgcatacgacaaaaactgcgtatagatcgaatgcagccttggattctaacgttatatacgaaggttttagcaactttatttattggccgactaccgaaagcgaacgaacaaacgtacgcattgctcaaaatgcggacctgtttaaaagctggggaatcaccacctttgaactggccccgcaatacaattcatcaaaagacggcacctttctggattcaattattgataacggctatgcatttaccgatcgttacgatctggggatgtctaccccgaacaaatacggctctgatgaggatctccgcaatgcgctacaagccttacacaaggccggactccaagcgattgctgactgggtgccggatcagatttataatctgccaggtaaggaagctgtgaccgtaacccgtagcgatgaccacggcaccacgtgggaagtctcaccgatcaagaacgttgtctacatcacgaataccattggtggcggtgaataccagaagaaatatggtggcgaatttttggacaccttgcaaaaagaatacccgcagctgttctcacaggtatacccagtgacccaaaccaccatcgatccgagcgtcaaaataaaagaatggagtgcaaagtattttaacggcacaaatattcttcatcggggcgcagggtacgtactccgtagcaatgacggcaaatactataatctcggaacctctactcagcagttcttgccgagccagctgtcagttcaagataacgagggatatgggtttgtcaaagaaggtaacaattatcattactatgatgagaataaacaaatggtaaaggacgcatttatccaggactctgttggtaattggtattactttgacaagaacggtaacatggtggcaaaccagtccccggtggaaatcagttcaaatggggcgtcgggcacgtatctcttcctgaataacggcacctccttccgtagtggcttagttaagaccgatgcaggaacctactactatgatggggacggtcgtatggtgcggaatcaaacggtttctgacggtgccatgacctacgtgttggacgaaaacggaaaactcgtgtccgaatcttttgatagttcggcgaccgaagcccacccacttaagccaggcgacctcaacggccagaagtaa

SEQ ID NO: 17 - protein, 1268 aa, synthetic construct
  REGION 1..1268: note = Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 18 - DNA, 3807 bp, synthetic construct
  misc_feature 1..3807: note = Synthetic
  source 1..3807: mol_type = other DNA
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattctgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttaggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 19 - protein, 1268 aa, synthetic construct
  REGION 1..1268: note = Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 20 - DNA, 3807 bp, synthetic construct
  misc_feature 1..3807: note = Synthetic
  source 1..3807: mol_type = other DNA
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattgcgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttaggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 21 - protein, 1268 aa, synthetic construct
  REGION 1..1268: note = Synthetic
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 22 - DNA, 3807 bp, synthetic construct
  misc_feature 1..3807: note = Synthetic
  source 1..3807: mol_type = other DNA
atgaactggcaaatcctgaaagaaatcctgggtaaaatgatcaaacaaaccaaagcgtcgtcgggcgttatctggaactccttcaaagaactggaagaatcagaactggaaaccgttattcgcgaaatcccggctccgtcgttcctgattccgctgccgaaacatctgaccgcgagcagcagcagcctgctggatcacgaccgtacggtctttcagtggctggatcagcaaccgccgtcatcggtgctgtatgtttcattcggtagcacctctgaagtcgatgaaaaagactttctggaaatcgctcgcggcctggtggatagtaaacagtccttcctgtgggtggttcgtccgggttttgtgaaaggcagcacgtgggttgaaccgctgccggatggcttcctgggtgaacgcggccgtattgtcaaatgggtgccgcagcaagaagtgctggcacatggtgctatcggcgcgttttggacccactctggttggaacagtacgctggaatccgtttgcgaaggtgtcccgatgattttcagcgattttggcctggaccagccgctgaatgcccgctatatgtctgatgttctgaaagtcggtgtgtacctggaaaacggttgggaacgtggcgaaattgcgaatgccatccgtcgcgttatggtcgatgaagaaggcgaatacattcgccagaacgctcgtgtcctgaaacaaaaagcggacgtgagcctgatgaaaggcggtagctcttatgaatcactggaatcgctggttagctacatcagttccctggaaaataaaaccgaaaccacggtgcgtcgccgtcgccgtattatcctgttcccggttccgtttcagggtcatattaacccgatcctgcaactggcgaatgttctgtattcaaaaggcttttcgatcaccatcttccatacgaacttcaacaaaccgaaaaccagtaactacccgcactttacgttccgctttattctggataacgacccgcaggatgaacgtatctccaatctgccgacccacggcccgctggccggtatgcgcattccgattatcaatgaacacggtgcagatgaactgcgccgtgaactggaactgctgatgctggccagtgaagaagatgaagaagtgtcctgtctgatcaccgacgcactgtggtatttcgcccagagcgttgcagattctctgaacctgcgccgtctggtcctgatgacgtcatcgctgttcaattttcatgcgcacgtttctctgccgcaatttgatgaactgggctacctggacccggatgacaaaacccgtctggaagaacaagccagtggttttccgatgctgaaagtcaaagacattaaatccgcctattcgggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 23 - protein, 1272 aa, synthetic construct
  REGION 1..1272: note = Synthetic
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 24 - DNA, 3819 bp, synthetic construct
  misc_feature 1..3819: note = Synthetic
  source 1..3819: mol_type = other DNA
atggattcgggttactcttcctcctatgcggcggctgcgggtatgcacgttgttatctgtccgtggctggcttttggtcacctgctgccgtgcctggatctggcacagcgtctggcttcacgcggccatcgtgtcagcttcgtgtctaccccgcgcaatatttcgcgtctgccgccggttcgtccggcactggctccgctggttgcatttgtcgctctgccgctgccgcgcgtggaaggtctgccggatggtgcggaaagtaccaacgacgtgccgcatgatcgcccggacatggttgaactgcaccgtcgtgcattcgatggtctggcagcaccgttttccgaatttctgggtacggcgtgcgccgattgggtgatcgttgacgtctttcatcactgggcggcggcggcggcgctggaacataaagttccgtgtgcaatgatgctgctgggctcagctcacatgattgcgtcgatcgcagaccgtcgcctggaacgtgcagaaaccgaaagtccggctgcggccggccagggtcgcccggcagctgcgccgaccttcgaagtggcccgcatgaaactgattcgtacgaaaggcagctctggtatgagcctggcagaacgctttagtctgaccctgtcccgtagttccctggtggttggtcgcagttgcgttgaatttgaaccggaaaccgtcccgctgctgtccacgctgcgtggtaaaccgatcacctttctgggtctgatgccgccgctgcatgaaggccgtcgcgaagatggtgaagacgcaacggtgcgttggctggatgcacagccggctaaaagcgtcgtgtatgtcgccctgggctctgaagtgccgctgggtgtggaaaaagttcacgaactggcactgggcctggaactggctggcacccgcttcctgtgggcactgcgtaaaccgacgggtgtgagcgatgcggacctgctgccggccggttttgaagaacgtacccgcggccgtggtgttgtcgcaacgcgttgggtcccgcaaatgagcattctggcgcatgccgcagtgggcgcctttctgacccactgtggttggaacagcacgatcgaaggcctgatgtttggtcacccgctgattatgctgccgatcttcggcgatcagggtccgaacgcacgtctgattgaagcgaaaaatgccggcctgcaagttgcgcgcaacgatggcgacggttctttcgaccgtgagggtgtggctgcggccattcgcgcagtggctgttgaagaagaatcatcgaaagtttttcaggcgaaagccaaaaaactgcaagaaatcgtcgcggatatggcctgccacgaacgctacattgatggtttcattcagcaactgcgctcctacaaagacggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 25 - protein, 1272 aa, synthetic construct
  REGION 1..1272: note = Synthetic
MGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 26 - DNA, 3819 bp, synthetic construct
  misc_feature 1..3819: note = Synthetic
  source 1..3819: mol_type = other DNA
atgggtagctcgggcatgtccctggcggaacgcttttcgctgacgctgagtcgctcatccctggttgttggtcgcagttgtgttgaatttgaaccggaaaccgttccgctgctgtctacgctgcgcggcaaaccgattaccttcctgggtctgatgccgccgctgcatgaaggccgtcgcgaagatggtgaagacgccacggtgcgttggctggatgctcagccggcgaaatcggtggtttatgtcgcactgggcagcgaagtgccgctgggtgtcgaaaaagtgcacgaactggccctgggcctggaactggcaggcacccgctttctgtgggcactgcgtaaaccgacgggcgttagcgatgctgacctgctgccggcgggtttcgaagaacgcacccgcggccgtggtgtcgtggccacccgttgggtgccgcaaatgtccattctggctcatgcggccgttggcgcatttctgacccactgcggttggaacagcacgatcgaaggcctgatgtttggtcatccgctgattatgctgccgatcttcggcgatcagggtccgaacgcacgcctgatcgaagccaaaaatgcaggcctgcaagttgcgcgtaacgatggcgacggtagctttgaccgcgaaggtgtcgcagctgcgattcgtgctgtggcggttgaagaagaaagcagcaaagtcttccaggccaaagcgaaaaaactgcaagaaatcgtggctgatatggcgtgtcatgaacgctatattgacggctttatccagcaactgcgttcttacaaagatgacagtggctatagttcctcatacgccgcagctgcgggtatgcatgttgtcatttgcccgtggctggcgtttggtcacctgctgccgtgtctggatctggcacagcgcctggcatctcgcggtcaccgtgtttcgttcgtcagcaccccgcgcaatatcagtcgtctgccgccggttcgtccggcgctggcgccgctggttgcgttcgttgcactgccgctgccgcgtgtggaaggtctgccggatggtgccgaatcgaccaacgacgttccgcatgatcgtccggacatggtcgaactgcatcgtcgcgcctttgatggcctggccgcaccgtttagcgaatttctgggtacggcctgcgcagattgggtcattgtggacgtttttcaccactgggcggcggcggcggcgctggaacataaagtgccgtgtgcgatgatgctgctgggttccgcccacatgattgcttcaatcgcggatcgtcgcctggaacgtgccgaaaccgaaagtccggcggcggcaggccagggtcgtccggcggcggcaccgacctttgaagtggcacgtatgaaactgattcgcacgaaaggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 27 - protein, 1269 aa, synthetic construct
  REGION 1..1269: note = Synthetic
MDGNSSSSPLHVVICPWLALGHLLPCLDIAERLASRGHRVSFVSTPRNIARLPPLRPAVAPLVDFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKAGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 28 - DNA, 3810 bp, synthetic construct
  misc_feature 1..3810: note = Synthetic
  source 1..3810: mol_type = other DNA
atggatggtaactcctcctcctcgccgctgcatgtggtcatttgtccgtggctggctctgggtcacctgctgccgtgtctggatattgctgaacgtctggcgtcacgcggccatcgtgtcagttttgtgtccaccccgcgcaacattgcccgtctgccgccgctgcgtccggctgttgcaccgctggttgatttcgtcgcactgccgctgccgcatgttgacggtctgccggagggtgcggaatcgaccaatgatgtgccgtatgacaaatttgaactgcaccgtaaggcgttcgatggtctggcggccccgtttagcgaatttctgcgtgcagcttgcgcagaaggtgcaggttctcgcccggattggctgattgtggacacctttcatcactgggcggcggcggcggcggtggaaaacaaagtgccgtgtgttatgctgctgctgggtgcagcaacggtgatcgctggtttcgcgcgtggtgttagcgaacatgcggcggcggcggtgggtaaagaacgtccggctgcggaagccccgagttttgaaaccgaacgtcgcaagctgatgaccacgcagaatgcctccggcatgaccgtggcagaacgctatttcctgacgctgatgcgtagcgatctggttgccatccgctcttgcgcagaatgggaaccggaaagcgtggcagcactgaccacgctggcaggtaaaccggtggttccgctgggtctgctgccgccgagtccggaaggcggtcgtggcgtttccaaagaagatgctgcggtccgttggctggacgcacagccggcaaagtcagtcgtgtacgtcgcactgggttcggaagtgccgctgcgtgcggaacaagttcacgaactggcactgggcctggaactgagcggtgctcgctttctgtgggcgctgcgtaaaccgaccgatgcaccggacgccgcagtgctgccgccgggtttcgaagaacgtacccgcggccgtggtctggttgtcacgggttgggtgccgcagattggcgttctggctcatggtgcggtggctgcgtttctgacccactgtggctggaactctacgatcgaaggcctgctgttcggtcatccgctgattatgctgccgatcagctctgatcagggtccgaatgcgcgcctgatggaaggccgtaaagtcggtatgcaagtgccgcgtgatgaatcagacggctcgtttcgtcgcgaagatgttgccgcaaccgtccgcgccgtggcagttgaagaagacggtcgtcgcgtcttcacggctaacgcgaaaaagatgcaagaaattgtggccgatggcgcatgccacgaacgttgtattgacggttttatccagcaactgcgcagttacaaggcgggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 29 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 30 - DNA, 2427 bp, Arabidopsis thaliana
  source 1..2427: mol_type = genomic DNA
atggcaaacgctgaacgtatgattacccgtgtccactcccaacgcgaacgcctgaacgaaaccctggtgtcggaacgcaacgaagttctggcactgctgagccgtgtggaagctaagggcaaaggtattctgcagcaaaaccagattatcgcggaatttgaagccctgccggaacaaacccgcaaaaagctggaaggcggtccgtttttcgatctgctgaaatctacgcaggaagcgatcgttctgccgccgtgggtcgcactggcagtgcgtccgcgtccgggcgtttgggaatatctgcgtgtcaacctgcatgcactggtggttgaagaactgcagccggctgaatttctgcacttcaaggaagaactggttgacggcgtcaaaaacggtaattttaccctggaactggattttgaaccgttcaatgccagtatcccgcgtccgacgctgcataaatatattggcaacggtgtggactttctgaatcgccatctgagcgcaaagctgttccacgataaagaatctctgctgccgctgctgaaattcctgcgtctgcatagtcaccagggcaagaacctgatgctgtccgaaaaaattcagaacctgaataccctgcaacacacgctgcgcaaggcggaagaatacctggccgaactgaaaagtgaaaccctgtacgaagaattcgaagcaaagttcgaagaaattggcctggaacgtggctggggtgacaatgctgaacgtgttctggatatgatccgtctgctgctggacctgctggaagcaccggacccgtgcaccctggaaacgtttctgggtcgcgtgccgatggttttcaacgtcgtgattctgtccccgcatggctattttgcacaggacaatgtgctgggttacccggataccggcggtcaggttgtctatattctggatcaagttcgtgcgctggaaattgaaatgctgcagcgcatcaagcagcaaggcctgaacatcaaaccgcgtattctgatcctgacccgtctgctgccggatgcagttggtaccacgtgcggtgaacgtctggaacgcgtctatgacagcgaatactgtgatattctgcgtgtcccgtttcgcaccgaaaagggtattgtgcgtaaatggatcagtcgcttcgaagtttggccgtatctggaaacctacacggaagatgcggccgtggaactgtccaaggaactgaatggcaaaccggacctgattatcggcaactatagcgatggtaatctggtcgcatctctgctggctcataaactgggtgtgacccagtgcacgattgcacacgctctggaaaagaccaaatatccggattcagacatctactggaaaaagctggatgacaaatatcatttttcgtgtcagttcaccgcggacatttttgccatgaaccacacggattttattatcaccagtacgttccaggaaatcgcgggctccaaagaaaccgtgggtcaatacgaatcacataccgccttcacgctgccgggcctgtatcgtgtggttcacggtatcgatgtttttgacccgaaattcaatattgtcagtccgggcgcggatatgtccatctattttccgtacaccgaagaaaagcgtcgcctgacgaaattccattcagaaattgaagaactgctgtactcggacgtggaaaacaaggaacacctgtgtgttctgaaagataaaaagaaaccgatcctgtttaccatggcccgtctggatcgcgtgaagaatctgtcaggcctggttgaatggtatggtaaaaacacgcgtctgcgcgaactggcaaatctggtcgtggttggcggtgaccgtcgcaaggaatcgaaagataacgaagaaaaggctgaaatgaagaaaatgtacgatctgatcgaagaatacaagctgaacggccagtttcgttggatcagctctcaaatggaccgtgtgcgcaatggcgaactgtatcgctacatttgcgataccaagggtgcgtttgttcagccggcactgtacgaagctttcggcctgaccgtcgtggaagccatgacgtgcggtctgccgacctttgcgacgtgtaaaggcggtccggccgaaattatcgtgcatggcaaatctggtttccatatcgatccgtatcacggtgatcaggcagctgacaccctggcggatttctttacgaagtgtaaagaagacccgtcacactgggatgaaatttcgaagggcggtctgcaacgtatcgaagaaaaatatacctggcagatttacagccaacgcctgctgaccctgacgggcgtctacggtttttggaaacatgtgtctaatctggatcgcctggaagcccgtcgctatctggaaatgttttacgcactgaagtatcgcccgctggcacaagccgttccgctggcacaggacgactaa

(19) United States
(12) Patent Application Publication
(10) Pub. No.: US 2024/0301462 A1
(43) Pub. Date: Sep. 12, 2024

(54) BIOSYNTHETIC PRODUCTION OF STEVIOL GLYCOSIDES

(71) Applicant: Conagen Inc., Bedford, MA (US)

(72) Inventors: Guohong Mao, Burlington, MA (US); Albertus Jan Vermeulen, Malden, MA (US); Shi Sun, Lexington, MA (US); Oliver Yu, Lexington, MA (US)

(73) Assignee: Conagen Inc., Bedford, MA (US)

(21) Appl. No.: 18/483,622

(22) Filed: Oct. 10, 2023

Related U.S. Application Data
(63) Continuation of application No. PCT/US22/23815, filed on Apr. 7, 2022.
(60) Provisional application No. 63/172,219, filed on Apr. 8, 2021.

(51) Int. Cl. C12P 19/56; C07H 13/08

(52) U.S. Cl. CPC C12P 19/56; C07H 13/08

(57) ABSTRACT

The present invention relates to novel steviol glycosides rebaudioside R6-5, rebaudioside R6-6, and rebaudioside R7-5 and the production of these novel steviol glycosides, such as through enzymatic bioconversion.

### RELATED APPLICATIONS

This application claims the benefit under 35 U.S.C. § 119(c) to U.S. Provisional Application No. 63/172,219, filed on Apr. 8, 2021 and entitled “BIOSYNTHETIC PRODUCTION OF STEVIOL GLYCOSIDES,” the entire contents of which are incorporated herein by reference.

### REFERENCE TO A SEQUENCE LISTING SUBMITTED AS A TEXT FILE

The instant application contains a Sequence Listing which has been submitted in ASCII format and is hereby incorporated by reference in its entirety. Said ASCII copy, created on Apr. 7, 2022, is named C149770046WO00-SEQ-ZJG and is 168,944 bytes in size.

### FIELD OF THE INVENTION

The field of the invention relates to methods and processes useful in the production of several specific steviol glycosides via enzymatic conversion as well as related compositions.

### BACKGROUND

Several steviol glycosides are found as compounds in Stevia rebaudiana leaves, and several of them have been widely used as high intensity, low-calorie sweeteners in food, feed and beverages. These naturally occurring steviol glycosides have the same basic diterpene structure (steviol backbone) but differ in the number and structure of their carbohydrate residue modifications (e.g. glucose, rhamnose, and xylose residues) at the C13 and C19 positions of the steviol backbone. Interestingly, these changes in sugar ‘ornamentation’ of the base steviol structure can affect the properties of the individual steviol glycosides themselves. These properties can include, without limitation: the taste profile, crystallization point, solubility, mouth feel and perceived sweetness among other differences. Steviol glycosides with known structures include stevioside, rebaudioside A, rebaudioside B, rebaudioside C, rebaudioside D, rebaudioside E, rebaudioside I, rebaudioside M, rebaudioside D3, rebaudioside N and rebaudioside O. In terms of commercial use rebaudiosides D and M have become generally regarded as safe (that is, it has ‘GRAS’ status) and are being studied for a wide range of uses in the food and beverage markets.

While consumers approve and actively seek natural and biological sources for food, feed, flavor, or medicinal components they are also concerned about sourcing, consistent taste profile and environmentally sustainable production. Microbial fermentation and production methods can provide rebaudiosides in quantities useful for a variety of industries and research while doing so in a more natural fashion than inorganic synthesis or current plant extraction techniques.

### SUMMARY

A need exists for the development of novel steviol glycoside variants as well as related production methods that can be performed economically and conveniently to further enable human and animal consumption.

The present disclosure, in some aspects, relate novel steviol glycosides (e.g., rebaudioside R6-5, rebaudioside R6-6, and rebaudioside R7-5) and methods of producing them. In some aspects, the present disclosure provides the use of rebaudioside R5-1 in the production of rebaudioside R6-5. In some aspects, the present disclosure provides the use of rebaudioside R6-5 in the production of rebaudioside R7-5. In some aspects, the present disclosure provides the use of rebaudioside R5-1 in the production of rebaudioside R6-6. In some aspects, the present disclosure provides the use of rebaudioside R5-1 for the production of a mixture of rebaudioside R6-5 and R7-5. In some aspects, the present disclosure provides the use of rebaudioside R5-1 for the production of a mixture of rebaudioside R6-5 and R6-6. In some aspects, the present disclosure provides the use of rebaudioside R5-1 for the production of a mixture of rebaudioside R6-5, R6-6, and R7-5. The product steviol glycosides were identified by NMR analysis and after production were subjected to various taste tests and processing tests to identify their particular flavor and performance characteristics.

Some aspects of the present disclosure provide a method of producing rebaudioside R6-6, the method comprising:
(I) preparing a reaction mixture comprising:
   (i) rebaudioside R5-1;
   (ii) one or more substrates selected from the group comprising of sucrose, uridine diphosphate (UDP), uridine diphosphate-glucose (UDP-glucose), and combinations thereof; and
   (iii) an enzyme selected from the group consisting of:
      (a) a UDP-glycosyltransferase (UGT);
      (b) a UDP-glycosyltransferase and a sucrose synthase; and
      (c) a UDP-glycosyltransferase fusion enzyme comprising a UDP-glycosyltransferase domain coupled to a sucrose synthase domain; and
(II) incubating the reaction mixture for a sufficient time to produce rebaudioside R6-5;
wherein the rebaudioside R5-1 has the structure of:

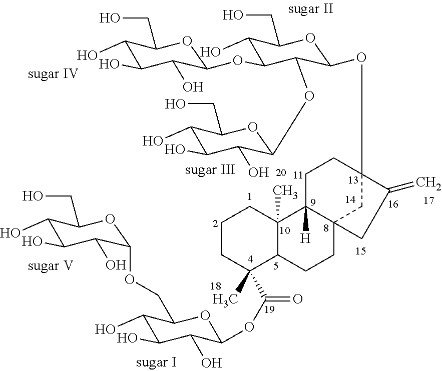

wherein the rebaudioside R5-1 has the structure of:

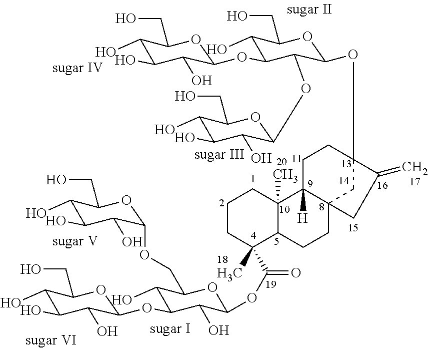

In some embodiments, a glucose is covalently coupled to sugar I of rebaudioside R5-1 by the enzyme to produce rebaudioside R6-6. In some embodiments, the UGT is at least 80% identical to the amino acid sequence of any one of SEQ ID NO: 9, 11, and 13. In some embodiments, the UGT comprises the amino acid sequence of any one of SEQ ID NOs: 9, 11, and 13. In some embodiments, the sucrose synthase or sucrose synthase domain is selected from the group consisting of Arabidopsis sucrose synthase I, an Arabidopsis sucrose synthase 3 and a Vigna radiate sucrose synthase. In some embodiments, the sucrose synthase or sucrose synthase domain is an Arabidopsis thaliana sucrose synthase I. In some embodiments, the sucrose synthase or sucrose synthase domain is at least 80% identical to the amino acid sequence of SEQ ID NO: 29. In some embodiments, the sucrose synthase or sucrose synthase domain comprises the amino acid sequence of SEQ ID NO: 29. In some embodiments, the UDP-glycosyltransferase fusion enzyme is at least 80% identical to the amino acid sequence of any one of SEQ ID NOs: 17, 19, and 21. In some embodiments, the UDP-glycosyltransferase fusion enzyme comprises the amino acid sequence of any one of SEQ ID NOs: 17, 19, and 21. In some embodiments, the methods further comprise producing rebaudioside R5-1 by incubating rebaudioside A with a glucansucrase and a substrate selected from the group consisting of sucrose, UDP, UDP-glucose, and combinations thereof. In some embodiments, a glucose is covalently coupled to sugar I of rebaudioside A by the glucansucrase to produce rebaudioside R5-1. In some embodiments, the glucansucrase comprises an amino acid sequence that is at least 80% identical to SEQ ID NO: 1 or 15. In some embodiments, the glucansucrase comprises the amino acid sequence of SEQ ID NO: 1 or SEQ ID NO: 15. In some embodiments, the reaction mixture is in vitro. In some embodiments, the reaction mixture is a cell-based reaction mixture. In some embodiments, the cell of the cell-based reaction mixture is selected from the group consisting of a yeast, a non-steviol glycoside producing plant, an alga, a fungus, and a bacterium.

Other aspects of the present disclosure provide a method of producing rebaudioside R7-5, the method comprising:
(I) preparing a reaction mixture comprising:
   (i) rebaudioside R6-5;
   (ii) one or more substrates selected from the group comprising of sucrose, uridine diphosphate (UDP), uridine diphosphate-glucose (UDP-glucose), and combinations thereof; and
   (iii) an enzyme selected from the group consisting of:
      (a) a UDP-glycosyltransferase (UGT);
      (b) a UDP-glycosyltransferase and a sucrose synthase; and
      (c) a UDP-glycosyltransferase fusion enzyme comprising a UDP-glycosyltransferase domain coupled to a sucrose synthase domain; and
(II) incubating the reaction mixture for a sufficient time to produce rebaudioside R7-5;
wherein the rebaudioside R6-5 has the structure of:

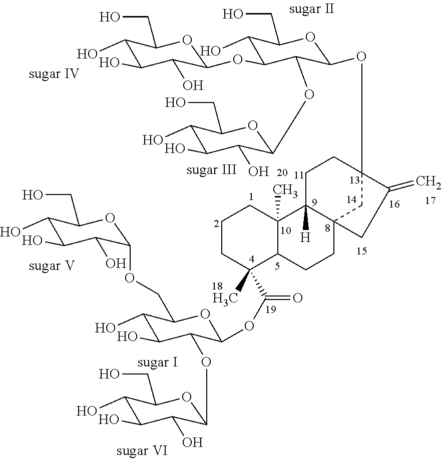

the rebaudioside R7-5 has the structure of:

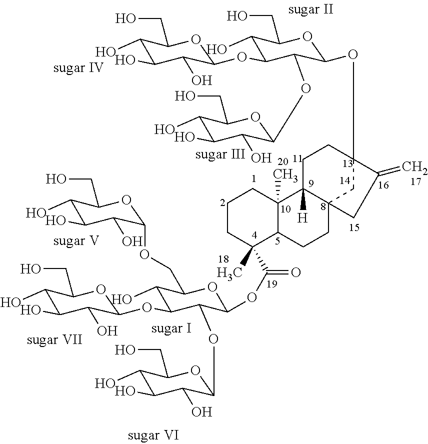

In some embodiments, a glucose is covalently coupled to sugar I of rebaudioside R6-5 by the enzyme to produce rebaudioside R7-5. In some embodiments, the UGT is at least 80% identical to the amino acid sequence of any one of SEQ ID NO: 9, 11, and 13. In some embodiments, the UGT comprises the amino acid sequence of any one of SEQ ID NOs: 9, 11, and 13. In some embodiments, the sucrose synthase or sucrose synthase domain is selected from the group consisting of Arabidopsis sucrose synthase I, an Arabidopsis sucrose synthase 3 and a Vigna radiate sucrose synthase. In some embodiments, the sucrose synthase or sucrose synthase domain is an Arabidopsis thaliana sucrose synthase I. In some embodiments, the sucrose synthase or sucrose synthase domain is at least 80% identical to the amino acid sequence of SEQ ID NO: 29. In some embodiments, the sucrose synthase or sucrose synthase domain comprises the amino acid sequence of SEQ ID NO: 29. In some embodiments, the UDP-glycosyltransferase fusion enzyme is at least 80% identical to the amino acid sequence of any one of SEQ ID NOs: 17, 19, and 21. In some embodiments, the UDP-glycosyltransferase fusion enzyme comprises the amino acid sequence of any one of SEQ ID NOs: 17, 19, and 21. In some embodiments, the methods further comprise producing rebaudioside R6-5 by incubating rebaudioside R5-1 with a second UGT and a substrate selected from the group consisting of sucrose, UDP, UDP-glucose, and a combination thereof. In some embodiments, a glucose is covalently coupled to sugar I of rebaudioside R5-1 by the second UGT to produce rebaudioside R6-5. In some embodiments, the second UGT is at least 80% identical to the amino acid sequence of any one of SEQ ID NOs: 3, 5, and 7. In some embodiments, the second UGT comprises the amino acid sequence of any one of SEQ ID NOs: 3, 5, and 7. In some embodiments, the method further comprises producing rebaudioside R5-1 by incubating rebaudioside A with a glucansucrase and a substrate selected from the group consisting of sucrose, UDP, UDP-glucose, and combinations thereof. In some embodiments, a glucose is covalently coupled to sugar I of rebaudioside A by the glucansucrase to produce rebaudioside R5-1. In some embodiments, the glucansucrase comprises an amino acid sequence that is at least 80% identical to SEQ ID NO: 1 or 15. In some embodiments, the glucansucrase comprises the amino acid sequence of SEQ ID NO: 1 or 15. In some embodiments, the reaction mixture is in vitro. In some embodiments, the reaction mixture is a cell-based reaction mixture. In some embodiments, the cell of the cell-based reaction mixture is selected from the group consisting of a yeast, a non-steviol glycoside producing plant, an alga, a fungus, and a bacterium.

Other aspects of the present disclosure provide a method of producing rebaudioside R6-5, the method comprising:
(I) preparing a reaction mixture comprising:
   (i) rebaudioside R5-1;
   (ii) one or more substrates selected from the group comprising of sucrose, uridine diphosphate (UDP), uridine diphosphate-glucose (UDP-glucose), and combinations thereof; and
   (iii) an enzyme selected from the group consisting of:
      (a) a UDP-glycosyltransferase (UGT);
      (b) a UDP-glycosyltransferase and a sucrose synthase; and
      (c) a UDP-glycosyltransferase fusion enzyme comprising a UDP-glycosyltransferase domain coupled to a sucrose synthase domain; and
(II) incubating the reaction mixture for a sufficient time to produce rebaudioside R6-5;
wherein the rebaudioside R5-1 has the structure of:

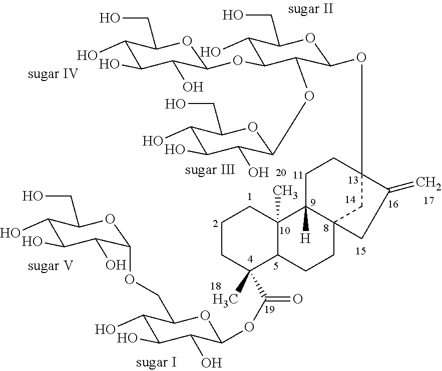

the rebaudioside R6-5 has the structure of:

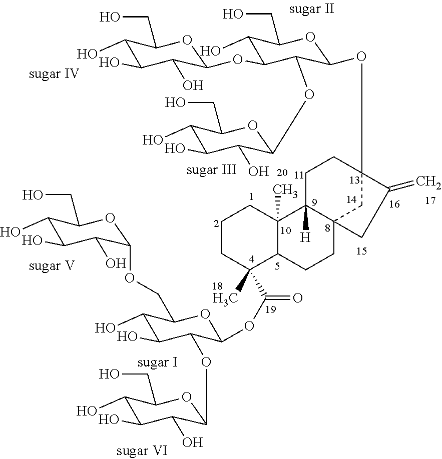

In some embodiments, a glucose is covalently coupled to sugar I of rebaudioside R5-1 by the enzyme to produce rebaudioside R6-5. In some embodiments, the UGT is at least 80% identical to the amino acid sequence of any one of SEQ ID NO: 3, 5, and 7. In some embodiments, the UGT comprises the amino acid sequence of any one of SEQ ID NOs: 3, 5, and 7. In some embodiments, the sucrose synthase or sucrose synthase domain is selected from the group consisting of Arabidopsis sucrose synthase I, an Arabidopsis sucrose synthase 3 and a Vigna radiate sucrose synthase. In some embodiments, the sucrose synthase or sucrose synthase domain is an Arabidopsis thaliana sucrose synthase I. In some embodiments, the sucrose synthase or sucrose synthase domain is at least 80% identical to the amino acid sequence of SEQ ID NO: 29. In some embodiments, the sucrose synthase or sucrose synthase domain comprises the amino acid sequence of SEQ ID NO: 29. In some embodiments, the UDP-glycosyltransferase fusion enzyme is at least 80% identical to the amino acid sequence of any one of SEQ ID NOs: 23, 25, and 27. In some embodiments, the UDP-glycosyltransferase fusion enzyme comprises the amino acid sequence of any one of SEQ ID NOs: 23, 25, and 27. In some embodiments, the method further comprises producing rebaudioside R5-1 by incubating rebaudioside A with a glucansucrase and a substrate selected from the group consisting of sucrose, UDP, UDP-glucose, and combinations thereof. In some embodiments, a glucose is covalently coupled to sugar I of rebaudioside A by the glucansucrase to produce rebaudioside R5-1. In some embodiments, the glucansucrase comprises an amino acid sequence that is at least 80% identical to SEQ ID NO: 1 or 15. In some embodiments, the glucansucrase comprises the amino acid sequence of SEQ ID NO: 1 or 15. In some embodiments, the reaction mixture is in vitro. In some embodiments, the reaction mixture is a cell-based reaction mixture. In some embodiments, the cell of the cell-based reaction mixture is selected from the group consisting of a yeast, a non-steviol glycoside producing plant, an alga, a fungus, and a bacterium.

Other aspects of the present disclosure provide a rebaudioside selected from;
(i) rebaudioside R6-5 having the structure:

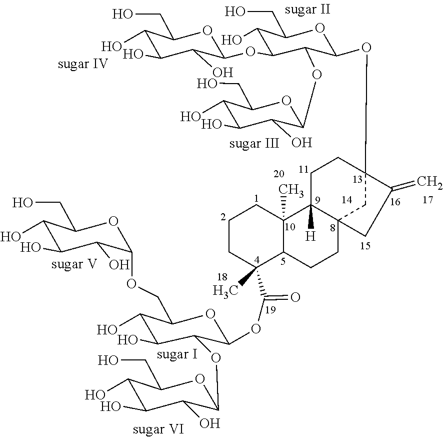

(ii) rebaudioside R6-6 having the structure:

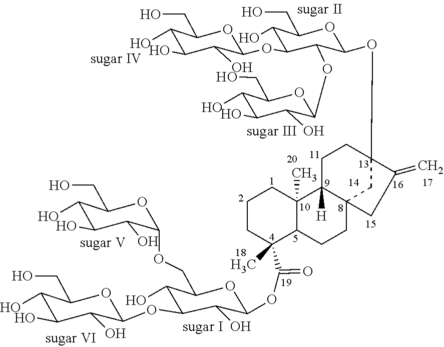

(iii) rebaudioside R7-5 having the structure:

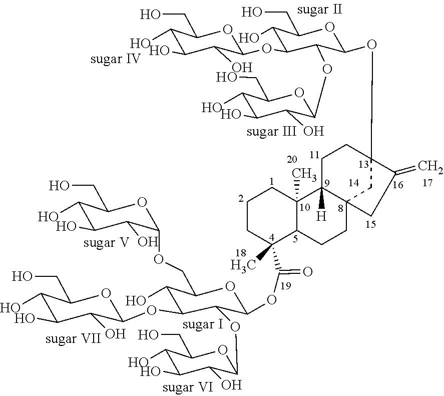

In some embodiments, the rebaudioside is a synthetic rebaudioside.

Other aspects of the present disclosure provide a composition comprising the rebaudioside described herein.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiments disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1. Biosynthetic pathway producing rebaudioside R6-5, rebaudioside R7-5, and rebaudioside R6-6.

FIGS. 2A-2B. IX14M catalysis reaction producing rebaudioside R5-1 from rebaudioside A. FIG. 2A shows the HPLC retention time of rebaudioside A and rebaudioside R5-1 at 0 hr following IX14M enzymatic conversion of rebaudioside A to rebaudioside R5-1. FIG. 2B shows the HPLC retention time of rebaudioside A and rebaudioside R5-1 at 3 hr following IX14M enzymatic conversion of rebaudioside A to rebaudioside R5-1.

FIGS. 3A-3D. UGT catalysis reaction producing rebaudioside R6-5 from rebaudioside R5-1. FIG. 3A shows the HPLC retention time of rebaudioside R5-1 at 20 hr following no addition of UGT enzymes. FIG. 3B shows the HPLC retention time of rebaudioside R5-1 and rebaudioside R6-5 at 20 hr following UGT (EU11) enzymatic conversion of rebaudioside R5-1 to rebaudioside R6-5. FIG. 3C shows the HPLC retention time of rebaudioside R5-1 and rebaudioside R6-5 at 20 hr following UGT (EUCP1) enzymatic conversion of rebaudioside R5-1 to rebaudioside R6-5. FIG. 3D shows the HPLC retention time of rebaudioside R5-1 and rebaudioside R6-5 at 20 hr following UGT (HV1) enzymatic conversion of rebaudioside R5-1 to rebaudioside R6-5.

FIGS. 4A-4D. UGT catalysis reaction producing rebaudioside R6-6 from rebaudioside R5-1. FIG. 4A shows the HPLC retention time of rebaudioside R5-1 at 20 hr following no addition of UGT enzymes. FIG. 4B shows the HPLC retention time of rebaudioside R5-1 and rebaudioside R6-6 at 20 hr following UGT (UGT76G1) enzymatic conversion of rebaudioside R5-1 to rebaudioside R6-6. FIG. 4C shows the HPLC retention time of rebaudioside R5-1 and rebaudioside R6-6 at 20 hr following UGT (LA) enzymatic conversion of rebaudioside R5-1 to rebaudioside R6-6. FIG. 4D shows the HPLC retention time of rebaudioside R5-1 and rebaudioside R6-6 at 20 hr following UGT (CP1) enzymatic conversion of rebaudioside R5-1 to rebaudioside R6-6.

FIGS. 5A-5C. Enzymatic catalysis reaction producing rebaudioside R7-5 from rebaudioside A. FIG. 5A shows the HPLC retention time of rebaudioside R5-1 at 3 hr following IX14M enzymatic conversion of rebaudioside A to rebaudioside R5-1. FIG. 5B shows the HPLC retention time of rebaudioside R6-5 at 20 hr following UGT (EUCP1) enzymatic conversion of rebaudioside R5-1 to rebaudioside R6-5. FIG. 5C shows the HPLC retention time of rebaudioside R6-5 and rebaudioside R7-5 at 20 hr following UGT (LA) enzymatic conversion of rebaudioside R6-5 to rebaudioside R7-5.

FIG. 6. The structure of rebaudioside R5-1.

FIG. 7. Key COSY/TOCSY and HMBC correlations of rebaudioside R5-1.

FIG. 8. The structure of rebaudioside R6-5.

FIG. 9. Key COSY/TOCSY and HMBC correlations of rebaudioside R6-5.

FIG. 10. The structure of rebaudioside R6-6.

FIG. 11. Key COSY/TOCSY and HMBC correlations of rebaudioside R6-6.

FIG. 12. The structure of rebaudioside R7-5.

FIG. 13. Key TOCSY and HMBC correlations of rebaudioside R7-5.

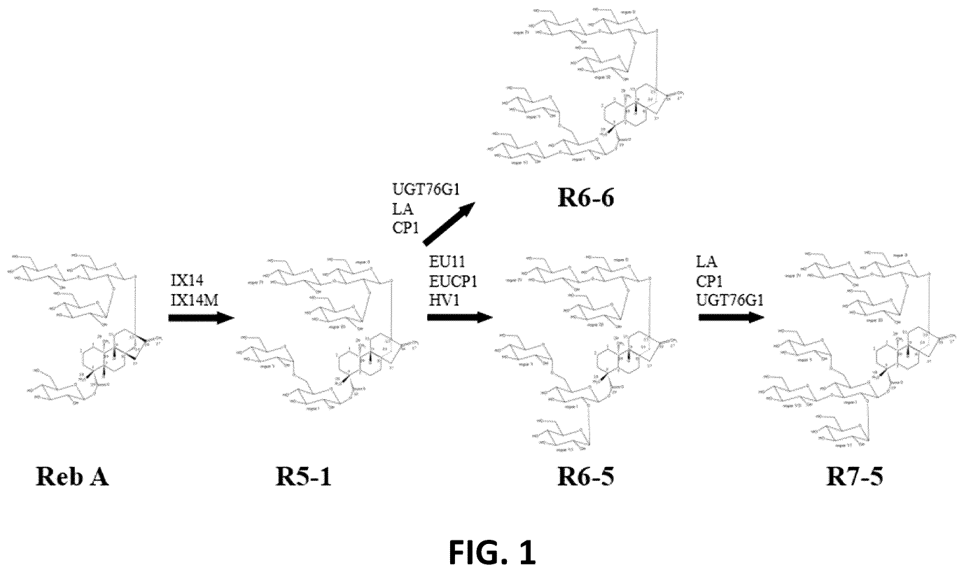

### DEFINITIONS

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

The terms “nucleic acid” and “nucleotide” are used according to their respective ordinary and customary meanings as understood by a person of ordinary skill in the art, and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is used according to its ordinary and customary meaning as understood by a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein refers to a process of mixing two or more chemical or biological entities (such as a chemical compound and an enzyme) and allowing them to interact under conditions favorable for producing a steviol glycoside composition.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide.” “protein,” and “peptide” are used according to their respective ordinary and customary meanings as understood by a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are used according to their ordinary and customary meanings to a person of ordinary skill in the art, and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full length polypeptide or protein, and has substantially the same biological activity, or carries out substantially the same function as the full length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions, and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from superfamilies and homologous polynucleotides or proteins from different species (Reeck et al., Cell 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical.

“Percent (%) amino acid sequence identity” with respect to the variant polypeptide sequences of the subject technology refers to the percentage of amino acid residues in a candidate sequence that are identical with the amino acid residues of a reference polypeptide after aligning the sequences and introducing gaps, if necessary, to achieve the maximum percent sequence identity.

Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, the % amino acid sequence identity may be determined using the sequence comparison program NCBI-BLAST2. The NCBI-BLAST2 sequence comparison program may be downloaded from ncbi.nlm.nih.gov. NCBI BLAST2 uses several search parameters, wherein all of those search parameters are set to default values including, for example, unmask yes, strand=all, expected occurrences 10, minimum low complexity length=15/5, multi-pass e-value=0.01, constant for multi-pass=25, dropoff for final gapped alignment=25 and scoring matrix=BLOSUM62. In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

In this sense, techniques for determining amino acid sequence “similarity” are well known in the art. In general, “similarity” refers to the exact amino acid to amino acid comparison of two or more polypeptides at the appropriate place, where amino acids are identical or possess similar chemical and/or physical properties such as charge or hydrophobicity. A so-termed “percent similarity” may then be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded therein, and comparing this to a second amino acid sequence. In general, “identity” refers to an exact nucleotide to nucleotide or amino acid to amino acid correspondence of two polynucleotides or polypeptide sequences, respectively. Two or more polynucleotide sequences can be compared by determining their “percent identity”, as can two or more amino acid sequences. The programs available in the Wisconsin Sequence Analysis Package, Version 8 (available from Genetics Computer Group, Madison, Wis.), for example, the GAP program, are capable of calculating both the identity between two polynucleotides and the identity and similarity between two polypeptide sequences, respectively. Other programs for calculating identity or similarity between sequences are known by those skilled in the art.

An amino acid position “corresponding to” a reference position refers to a position that aligns with a reference sequence, as identified by aligning the amino acid sequences. Such alignments can be done by hand or by using well-known sequence alignment programs such as ClustalW2, Blast 2, etc.

Unless specified otherwise, the percent identity of two polypeptide or polynucleotide sequences refers to the percentage of identical amino acid residues or nucleotides across the entire length of the shorter of the two sequences.

“Coding sequence” is used according to its ordinary and customary meaning as understood by a person of ordinary skill in the art, and is used without limitation to refer to a DNA sequence that encodes for a specific amino acid sequence.

“Suitable regulatory sequences” is used according to its ordinary and customary meaning as understood by a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is used according to its ordinary and customary meaning as understood by a person of ordinary skill in the art, and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is used according to its ordinary and customary meaning as understood by a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is used according to its ordinary and customary meaning as understood by a person of ordinary skill in the art, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “recombinant” or “transformed” organisms.

The terms “transformed,” “transgenic.” and “recombinant,” when used herein in connection with host cells, are used according to their ordinary and customary meanings as understood by a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are used according to their ordinary and customary meanings as understood by a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are used according to their ordinary and customary meanings as understood by a person of ordinary skill in the art, and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Standard recombinant DNA and molecular cloning techniques used herein are well known in the art and are described, for example, by Sambrook, J., Fritsch, E. F. and Maniatis, T. Molecular Cloning: A Laboratory Manual, 2^{nd} ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-Interscience, 1987; the entireties of each of which are hereby incorporated herein by reference to the extent they are consistent herewith.

As used herein, “synthetic” or “organically synthesized” or “chemically synthesized” or “organically synthesizing” or “chemically synthesizing” or “organic synthesis” or “chemical synthesis” are used to refer to preparing the compounds through a series of chemical reactions; this does not include extracting the compound, for example, from a natural source.

As used herein, the term “stereoisomer” is a general term for all isomers of individual molecules that differ only in the orientation of their atoms in space. “Stereoisomer” includes enantiomers and isomers of compounds with more than one chiral center that are not mirror images of one another (diastereomers).

Cellular system is any cells that provide for the expression of ectopic proteins. It included bacteria, yeast, plant cells and animal cells. It includes both prokaryotic and eukaryotic cells. It also includes the in vitro expression of proteins based on cellular components, such as ribosomes.

Growing the Cellular System. Growing includes providing an appropriate medium that would allow cells to multiply and divide. It also includes providing resources so that cells or cellular components can translate and make recombinant proteins.

Protein Expression. Protein production can occur after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration”. Any embodiment described herein as “exemplary” is not necessarily to be construed as preferred or advantageous over other embodiments.

### DETAILED DESCRIPTION

The present disclosure relates, at least in part, to novel steviol glycosides, rebaudioside R6-5, rebaudioside R6-6, and rebaudioside R7-5, and methods of producing these novel steviol glycosides, such as through enzymatic conversion. The chemical structures can be confirmed by LC MS and NMR analysis. Rebaudioside R6-5 and rebaudioside R6-6 contain 6 glucosyl groups and rebaudioside R7-5 contains 7 glucosyl groups. In some embodiments, the rebaudioside R6-5, rebaudioside R6-6, or rebaudioside R7-5 can be used in food and beverages.

### Rebaudioside R6-5 and Methods of Producing

Some aspects of the present disclosure provide a compound that has been given the name “Rebaudioside R6-5 (Reb R6-5).” The Reb 6-5 compound is a steviol glycoside with glucosyl moieties covalently bound at the C-13 hydroxyl in the form of an ether and at C-19 as an ester.

Rebaudioside R6-5 has the molecular formula of C₅₆H₉₀O₃₃ and has the structure of:

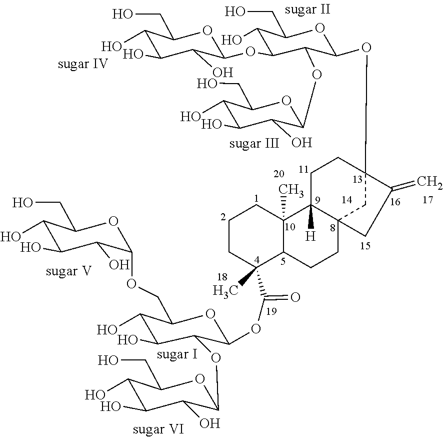

Some aspects of the present disclosure provide methods of producing rebaudioside R6-5. In some embodiments of any one of the methods provided, the rebaudioside R6-5 is produced from one or more of rebaudioside A (Reb A) and rebaudioside R5-1 (Reb R5-1). A glucose can be covalently coupled to rebaudioside A (e.g., to sugar I of rebaudioside A) by a glucansucrase (e.g., IX14 or IX14M) to produce rebaudioside R5-1. The enzymes EU11, EUCP1, or HV1 can further covalently couple a second glucose to rebaudioside R5-1 (e.g., to sugar I of rebaudioside R5-1), yielding rebaudioside R6-5. Rebaudioside R5-1 has the structure of:

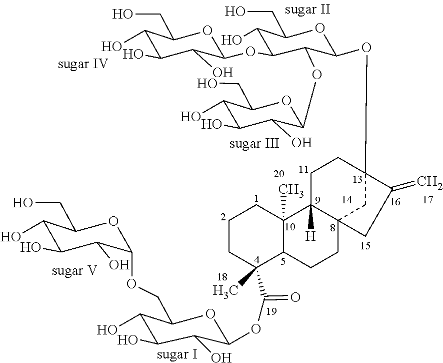

In some embodiments, the method of producing rebaudioside R6-5 comprises (I) preparing a reaction mixture comprising: (i) rebaudioside R5-1; (ii) one or more substrates selected from the group consisting of sucrose, uridine diphosphate (UDP), uridine diphosphate-glucose (UDP-glucose), and combinations thereof; and (iii) an enzyme selected from the group consisting of: (a) a UDP-glycosyltransferase (UGT); (b) a UDP-glycosyltransferase and a sucrose synthase separately added to the reaction mixture; and (c) a UDP-glycosyltransferase fusion enzyme comprising a UDP-glycosyltransferase domain coupled to a sucrose synthase domain; and (II) incubating the reaction mixture for a sufficient time to produce rebaudioside R6-5.

In some embodiments of any one of the methods or compositions provided herein, the enzyme for producing a rebaudioside R6-5 from rebaudioside R5-1 comprises an EU11 enzyme. The EU11 enzyme is an UDP-glycosyltransferase (UGT). In some embodiments of any one of the methods or compositions provided herein, the EU11 comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 3. In some embodiments of any one of the methods or compositions provided herein, the EU11 comprises an amino acid sequence at least 70%, 75%, 80%, 85%, 90%, 95%, or 99% identical to the amino acid sequence of SEQ ID NO: 3. In some embodiments of any one of the methods or compositions provided herein, the EU11 comprises the amino acid sequence of SEQ ID NO: 3. In some embodiments of any one of the methods or compositions provided herein, the EU11 consists essentially of or consists of the amino acid sequence of SEQ ID NO: 3.

In some embodiments of any one of the methods or compositions provided herein, the enzyme for producing a rebaudioside R6-5 from rebaudioside 5-1 comprises an EUCP1 enzyme. The EUCP1 enzyme is an UDP-glycosyltransferase (UGT). In some embodiments of any one of the methods or compositions provided herein, the EUCP1 comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 5. In some embodiments of any one of the methods or compositions provided herein, the EU11 comprises an amino acid sequence at least 70%, 75%, 80%, 85%, 90%, 95%, or 99% identical to the amino acid sequence of SEQ ID NO: 5. In some embodiments of any one of the methods or compositions provided herein, the EUCP1 comprises the amino acid sequence of SEQ ID NO: 5. In some embodiments of any one of the methods or compositions provided herein, the EUCP1 consists essentially of or consists of the amino acid sequence of SEQ ID NO: 5.

In some embodiments of any one of the methods or compositions provided herein, the enzyme for producing a rebaudioside R6-5 from rebaudioside 5-1 comprises an HV1 enzyme. The HV1 enzyme is an UDP-glycosyltransferase (UGT). In some embodiments of any one of the methods or compositions provided herein, the HV1 comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 7. In some embodiments of any one of the methods or compositions provided herein, the EU11 comprises an amino acid sequence at least 70%, 75%, 80%, 85%, 90%, 95%, or 99% identical to the amino acid sequence of SEQ ID NO: 7. In some embodiments of any one of the methods or compositions provided herein, the HV1 comprises the amino acid sequence of SEQ ID NO: 7. In some embodiments of any one of the methods or compositions provided herein, the HV1 consists essentially of or consists of the amino acid sequence of SEQ ID NO: 7.

In some embodiments of any one of the methods or compositions provided herein, the enzyme for producing a rebaudioside R6-5 from rebaudioside 5-1 comprises a UDP-glycosyltransferase fusion enzyme comprising a UDP-glycosyltransferase domain coupled to a sucrose synthase domain. The fusion enzyme has the activity of the UDP-glycosyltransferase and sucrose synthase activity. In some embodiments of any one of the methods or compositions provided herein, the UDP-glycosyltransferase domain in the fusion enzyme EU11, EUCP1, or HV1, and the sucrose synthase domain is any one of the sucrose synthase (e.g., an Arabidopsis sucrose synthase I, an Arabidopsis sucrose synthase 3, or a Vigna radiate sucrose synthase) and variants as described herein.

In some embodiments of any one of the methods or compositions provided herein, the sucrose synthase domain comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 29. In some embodiments of any one of the methods or compositions provided herein, the sucrose synthase domain comprises an amino acid sequence at least 70%, 75%, 80%, 85%, 90%, 95%, or 99% identical to the amino acid sequence of SEQ ID NO: 29. In some embodiments of any one of the methods or compositions provided herein, the sucrose synthase domain comprises the amino acid sequence of SEQ ID NO: 29. In some embodiments of any one of the methods or compositions provided herein, the sucrose synthase domain consists essentially of or consists of the amino acid sequence of SEQ ID NO: 29.

In some embodiments of any one of the methods or compositions provided herein, the fusion enzyme comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of any one of SEQ ID NOs:23, 25, or 27. In some embodiments of any one of the methods or compositions provided herein, the fusion enzyme comprises an amino acid sequence at least? 70%, 75%, 80%, 85%, 90%, 95%, or 99% identical to the amino acid sequence of any one of SEQ ID NOs: 23, 25, or 27. In some embodiments of any one of the methods or compositions provided herein, the fusion enzyme comprises the amino acid sequence of any one of SEQ ID NOs: 23, 25, or 27. In some embodiments of any one of the methods or compositions provided herein, the fusion enzyme consists essentially of or consists of the amino acid sequence of any one of SEQ ID NOs: 23, 25, or 27.

In some embodiments of any one of the methods or compositions provided herein, the methods of producing rebaudioside R6-5 described herein further comprises a step of producing rebaudioside R5-1. In some embodiments, Rebaudioside R5-1 can be produced by incubating rebaudioside A with a glucansucrase and a substrate selected from the group consisting of sucrose, UDP, UDP-glucose, and combinations thereof. In some embodiments of any one of the methods or compositions provided herein, the glucansucrase used to produce rebaudioside R5-1 from rebaudioside A is IX14 or IX14M.

In some embodiments of any one of the methods or compositions provided herein, the enzyme for producing a rebaudioside 5-1 from rebaudioside A comprises a glucansucrase (e.g., an IX14 enzyme or an IX14M enzyme). In some embodiments of any one of the methods or compositions provided herein, the IX14 comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 15. In some embodiments of any one of the methods or compositions provided herein, the IX14 comprises an amino acid sequence at least 70%, 75%, 80%, 85%, 90%, 95%, or 99% identical to the amino acid sequence of SEQ ID NO: 15. In some embodiments of any one of the methods or compositions provided herein, the IX14 comprises the amino acid sequence of SEQ ID NO: 15. In some embodiments of any one of the methods or compositions provided herein, the IX14 consists essentially of or consists of the amino acid sequence of SEQ ID NO: 15.

In some embodiments of any one of the methods or compositions provided herein, the IX14M comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 1. In some embodiments of any one of the methods or compositions provided herein, the IX14M comprises an amino acid sequence at least 70%, 75%, 80%, 85%, 90%, 95%, or 99% identical to the amino acid sequence of SEQ ID NO: 1. In some embodiments of any one of the methods or compositions provided herein, the IX14 comprises the amino acid sequence of SEQ ID NO: 1. In some embodiments of any one of the methods or compositions provided herein, the IX14M consists essentially of or consists of the amino acid sequence of SEQ ID NO: 1.

In some embodiments, any one of the methods of producing rebaudioside R6-5 described herein further comprises isolating the produced rebaudioside R6-5.

### Rebaudioside R6-6 and Methods of Producing

Some aspects of the present disclosure provide a compound that has been given the name “Rebaudioside R6-6 (Reb R6-6).” The Reb 6-6 compound is a steviol glycoside with glucosyl moieties covalently bound at the C-13 hydroxyl in the form of an ether and at C-19 as an ester.

Rebaudioside R6-6 has the molecular formula of C₅₆H₉₀O₃₃ and has the structure of:

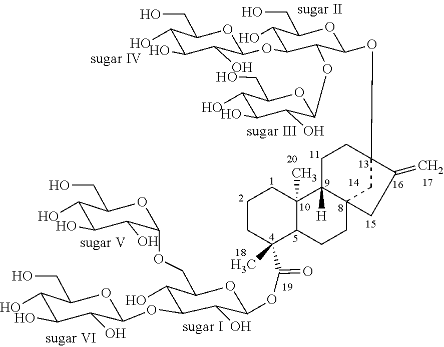

Some aspects of the present disclosure provide methods of producing rebaudioside R6-6. In some embodiments of any one of the methods provided, the rebaudioside R6-6 is produced from one or more of rebaudioside A (Reb A) and rebaudioside R5-1 (Reb R5-1). A glucose can be covalently coupled to rebaudioside (e.g., to sugar I of rebaudioside A) by a glucansucrase (e.g., IX14 or IX14M) to produce rebaudioside R5-1. The enzymes UGT76G1 and its variants (e.g., L200A, and CP1 variants) can further covalently couple a second glucose to rebaudioside R5-1 (e.g., to sugar I of rebaudioside R5-1), yielding rebaudioside R6-6.

In some embodiments of any one of the methods provided, the method comprises (I) preparing a reaction mixture comprising: (i) rebaudioside R5-1; (ii) one or more substrates selected from the group consisting of sucrose, uridine diphosphate (UDP), uridine diphosphate-glucose (UDP-glucose), and combinations thereof; and (iii) an enzyme selected from the group consisting of: (a) a UDP-glycosyltransferase (UGT); (b) a UDP-glycosyltransferase and a sucrose synthase separately added to the reaction mixture; and (b) a UDP-glycosyltransferase fusion enzyme comprising a UDP-glycosyltransferase domain coupled to a sucrose synthase domain; and (II) incubating the reaction mixture for a sufficient time to produce rebaudioside R6-6.

In some embodiments of any one of the methods or compositions provided herein, the enzyme for producing a rebaudioside R6-6 from rebaudioside R5-1 comprises a UDP-glycosyltransferase (UGT). In some embodiments of any one of the methods or compositions provided herein, the UGT is a uridine diphospho glycosyltransferase (e.g., UGT76G1 or a functional variant thereof). UGT76G1 is a UGT with a 1,3-13-O-glucose glycosylation activity. It has also been shown that UGT76G1 has 1,3-19-O-glucose glycosylation activity. In some embodiments of any one of the methods or compositions provided herein, the UGT76G1 comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 9. In some embodiments of any one of the methods or compositions provided herein, the UGT76G1 comprises an amino acid sequence at least 70%, 75%, 80%, 85%, 90%, 95%, or 99% identical to the amino acid sequence of SEQ ID NO: 9. In some embodiments of any one of the methods or compositions provided herein, the UGT76G1 comprises the amino acid sequence of SEQ ID NO: 9. In some embodiments of any one of the methods or compositions provided herein, the UGT76G1 consists essentially of or consists of the amino acid sequence of SEQ ID NO: 9.

In some embodiments of any one of the methods or compositions provided herein, the enzyme for producing a rebaudioside R6-6 from rebaudioside 5-1 comprises a UGT76G1 L200A variant (also referred to herein as “LA”). In some embodiments of any one of the methods or compositions provided herein, the UGT76G1 L200A variant comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 11. In some embodiments of any one of the methods or compositions provided herein, the UGT76G1 L200A variant comprises an amino acid sequence at least 70%, 75%, 80%, 85%, 90%, 95%, or 99% identical to the amino acid sequence of SEQ ID NO: 11. In some embodiments of any one of the methods or compositions provided herein, the UGT76G1 L200A variant comprises the amino acid sequence of SEQ ID NO: 11. In some embodiments of any one of the methods or compositions provided herein, the UGT76G1 L200A variant consists essentially of or consists of the amino acid sequence of SEQ ID NO: 11.

In some embodiments of any one of the methods or compositions provided herein, the enzyme for producing a rebaudioside R6-6 from rebaudioside 5-1 comprises an a UGT76G1 circular permutation (CP1) variant (also referred to herein as “CP1”). In some embodiments of any one of the methods or compositions provided herein, the UGT76G1 CP1 variant comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 13. In some embodiments of any one of the methods or compositions provided herein, the UGT76G1 CP1 variant comprises an amino acid sequence at least 70%, 75%, 80%, 85%, 90%, 95%, or 99% identical to the amino acid sequence of SEQ ID NO: 13. In some embodiments of any one of the methods or compositions provided herein, the UGT76G1 CP1 variant comprises the amino acid sequence of SEQ ID NO: 13. In some embodiments of any one of the methods or compositions provided herein, the UGT76G1 CP1 variant consists essentially of or consists of the amino acid sequence of SEQ ID NO: 13.

In some embodiments of any one of the methods or compositions provided herein, the enzyme for producing a rebaudioside R6-6 from rebaudioside 5-1 comprises a UDP-glycosyltransferase fusion enzyme comprising a UDP-glycosyltransferase domain coupled to a sucrose synthase domain. The fusion enzyme has the activity of the UDP-glycosyltransferase and sucrose synthase activity. In some embodiments of any one of the methods or compositions provided herein, the UDP-glycosyltransferase domain in the fusion enzyme is any one of the UGT76G1 and variants (e.g., LA or CP1) as described herein, and the sucrose synthase domain is any one of the sucrose synthase (e.g., an Arabidopsis sucrose synthase I, an Arabidopsis sucrose synthase 3, or a Vigna radiate sucrose synthase) and variants as described herein.

In some embodiments of any one of the methods or compositions provided herein, the sucrose synthase domain comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 29. In some embodiments of any one of the methods or compositions provided herein, the sucrose synthase domain comprises an amino acid sequence at least 70%, 75%, 80%, 85%, 90%, 95%, or 99% identical to the amino acid sequence of SEQ ID NO: 29. In some embodiments of any one of the methods or compositions provided herein, the sucrose synthase domain comprises the amino acid sequence of SEQ ID NO: 29. In some embodiments of any one of the methods or compositions provided herein, the sucrose synthase domain consists essentially of or consists of the amino acid sequence of SEQ ID NO: 29.

In some embodiments of any one of the methods or compositions provided herein, the fusion enzyme comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of any one of SEQ ID NOs: 17, 19, or 21. In some embodiments of any one of the methods or compositions provided herein, the fusion enzyme comprises an amino acid sequence at least 70%, 75%, 80%, 85%, 90%, 95%, or 99% identical to the amino acid sequence of any one of SEQ ID NOs: 17, 19, or 21. In some embodiments of any one of the methods or compositions provided herein, the fusion enzyme comprises the amino acid sequence of any one of SEQ ID NOs: 17, 19, or 21. In some embodiments of any one of the methods or compositions provided herein, the fusion enzyme consists essentially of or consists of the amino acid sequence of any one of SEQ ID NOs: 17, 19, or 21.

In some embodiments of any one of the methods or compositions provided herein, the methods of producing rebaudioside R6-6 described herein further comprises a step of producing rebaudioside R5-1. In some embodiments, Rebaudioside R5-1 can be produced using any one of the methods of producing rebaudioside R5-1 described herein. In some embodiments, Rebaudioside R5-1 can be produced by incubating rebaudioside A with a glucansucrase and a substrate selected from the group consisting of sucrose, UDP, UDP-glucose, and combinations thereof. In some embodiments of any one of the methods or compositions provided herein, the glucansucrase used to produce rebaudioside R5-1 from rebaudioside A is IX14 or IX14M.

In some embodiments, any one of the methods of producing rebaudioside R6-6 described herein further comprises isolating the produced rebaudioside R6-6.

### Rebaudioside R7-5 and Methods of Producing

Some aspects of the present disclosure provide a compound that has been given the name “Rebaudioside R7-5 (Reb R7-5).” The Reb 7-5 compound is a steviol glycoside with glucosyl moieties covalently bound at the C-13 hydroxyl in the form of an ether and at C-19 as an ester.

Rebaudioside R7-5 has the molecular formula of C₆₂H₉₉O₃₈ and has the structure of:

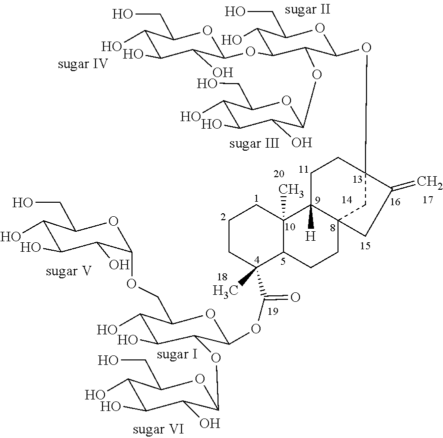

Some aspects of the present disclosure provide methods of producing rebaudioside R7-5. In some embodiments of any one of the methods provided, the rebaudioside R7-5 is produced from one or more of rebaudioside A (Reb A), rebaudioside R5-1 (Reb R5-1), and rebaudioside R6-5. A glucose can be covalently coupled to the rebaudioside A (e.g., to sugar I of rebaudioside A) by a glucansucrase (e.g., IX14 or IX14M) to produce rebaudioside R5-1. The enzymes EU11, EUCP1, and HV1 can further covalently couple a second glucose to rebaudioside R5-1 (e.g., to sugar I of rebaudioside R5-1), yielding rebaudioside R6-5. The enzymes UGT76G1 and its variants (e.g., L200A, and CP1 variants) can further covalently couple a third glucose to rebaudioside R6-5 (e.g., to sugar I of rebaudioside R6-5), yielding rebaudioside R7-5.

In some embodiments of any one of the methods provided, the method comprises (I) preparing a reaction mixture comprising: (i) rebaudioside R6-5; (ii) one or more substrates selected from the group consisting of sucrose, uridine diphosphate (UDP), uridine diphosphate-glucose (UDP-glucose), and combinations thereof; and (iii) an enzyme selected from the group consisting of: (a) a UDP-glycosyltransferase (UGT); (b) a UDP-glycosyltransferase and a sucrose synthase separately added to the reaction mixture; and (c) a UDP-glycosyltransferase fusion enzyme comprising a UDP-glycosyltransferase domain coupled to a sucrose synthase domain; and (II) incubating the reaction mixture for a sufficient time to produce rebaudioside R7-5.

In some embodiments of any one of the methods or compositions provided herein, the enzyme for producing a rebaudioside R7-5 from rebaudioside R6-5 comprises a UDP-glycosyltransferase (UGT). In some embodiments of any one of the methods or compositions provided herein, the UGT is a uridine diphospho glycosyltransferase (e.g., UGT76G1 or a functional variant thereof). UGT76G1 is a UGT with a 1,3-13-O-glucose glycosylation activity. It has also been shown that UGT76G1 has 1,3-19-O-glucose glycosylation activity. In some embodiments of any one of the methods or compositions provided herein, the UGT76G1 comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 9. In some embodiments of any one of the methods or compositions provided herein, the UGT76G1 comprises an amino acid sequence at least 70%, 75%, 80%, 85%, 90%, 95%, or 99% identical to the amino acid sequence of SEQ ID NO: 9. In some embodiments of any one of the methods or compositions provided herein, the UGT76G1 comprises the amino acid sequence of SEQ ID NO: 9. In some embodiments of any one of the methods or compositions provided herein, the UGT76G1 consists essentially of or consists of the amino acid sequence of SEQ ID NO: 9.

In some embodiments of any one of the methods or compositions provided herein, the enzyme for producing a rebaudioside R7-5 from rebaudioside R6-5 comprises a UGT76G1 L200A variant (also referred to herein as “LA”). In some embodiments of any one of the methods or compositions provided herein, the UGT76G1 L200A variant comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 11. In some embodiments of any one of the methods or compositions provided herein, the UGT76G1 L200A variant comprises an amino acid sequence at least 70%, 75%, 80%, 85%, 90%, 95%, or 99% identical to the amino acid sequence of SEQ ID NO: 11. In some embodiments of any one of the methods or compositions provided herein, the UGT76G1 L200A variant comprises the amino acid sequence of SEQ ID NO: 11. In some embodiments of any one of the methods or compositions provided herein, the UGT76G1 L200A variant consists essentially of or consists of the amino acid sequence of SEQ ID NO: 11.

In some embodiments of any one of the methods or compositions provided herein, the enzyme for producing a rebaudioside R7-5 from rebaudioside R6-5 comprises an a UGT76G1 circular permutation (CP1) variant (also referred to herein as “CP1”). In some embodiments of any one of the methods or compositions provided herein, the UGT76G1 CP1 variant comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 13. In some embodiments of any one of the methods or compositions provided herein, the UGT76G1 CP1 variant comprises an amino acid sequence at least 70%, 75%, 80%, 85%, 90%, 95%, or 99% identical to the amino acid sequence of SEQ ID NO: 13. In some embodiments of any one of the methods or compositions provided herein, the UGT76G1 CP1 variant comprises the amino acid sequence of SEQ ID NO: 13. In some embodiments of any one of the methods or compositions provided herein, the UGT76G1 CP1 variant consists essentially of or consists of the amino acid sequence of SEQ ID NO: 13.

In some embodiments of any one of the methods or compositions provided herein, the enzyme for producing a rebaudioside R7-5 from rebaudioside R6-5 comprises a UDP-glycosyltransferase fusion enzyme comprising a UDP-glycosyltransferase domain coupled to a sucrose synthase domain. The fusion enzyme has the activity of the UDP-glycosyltransferase and sucrose synthase activity. In some embodiments of any one of the methods or compositions provided herein, the UDP-glycosyltransferase domain in the fusion enzyme is any one of the UGT76G1 and variants (e.g., LA or CP1) as described herein, and the sucrose synthase domain is any one of the sucrose synthase (e.g., an Arabidopsis sucrose synthase I, an Arabidopsis sucrose synthase 3, or a Vigna radiate sucrose synthase) and variants as described herein.

In some embodiments of any one of the methods or compositions provided herein, the sucrose synthase domain comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of SEQ ID NO: 29. In some embodiments of any one of the methods or compositions provided herein, the sucrose synthase domain comprises an amino acid sequence at least 70%, 75%, 80%, 85%, 90%, 95%, or 99% identical to the amino acid sequence of SEQ ID NO: 29. In some embodiments of any one of the methods or compositions provided herein, the sucrose synthase domain comprises the amino acid sequence of SEQ ID NO: 29. In some embodiments of any one of the methods or compositions provided herein, the sucrose synthase domain consists essentially of or consists of the amino acid sequence of SEQ ID NO: 29.

In some embodiments of any one of the methods or compositions provided herein, the fusion enzyme comprises an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical to the amino acid sequence of any one of SEQ ID NOs: 17, 19, or 21. In some embodiments of any one of the methods or compositions provided herein, the fusion enzyme comprises an amino acid sequence at least 70%, 75%, 80%, 85%, 90%, 95%, or 99% identical to the amino acid sequence of any one of SEQ ID NOs: 17, 19, or 21. In some embodiments of any one of the methods or compositions provided herein, the fusion enzyme comprises the amino acid sequence of any one of SEQ ID NOs: 17, 19, or 21. In some embodiments of any one of the methods or compositions provided herein, the fusion enzyme consists essentially of or consists of the amino acid sequence of any one of SEQ ID NOs: 17, 19, or 21.

In some embodiments of any one of the methods or compositions provided herein, the methods of producing rebaudioside R7-5 described herein further comprises a step of producing rebaudioside R6-5. In some embodiments, Rebaudioside 6-5 can be produced using any one of the methods of producing R6-5 described herein. In some embodiments, Rebaudioside 6-5 can be produced by incubating rebaudioside R5-1 with a UDP-glycotransferase and a substrate selected from the group consisting of sucrose, UDP, UDP-glucose, and combinations thereof. In some embodiments of any one of the methods or compositions provided herein, the UDP-glycotransferases used to produce rebaudioside 6-5 from rebaudioside R5-1 are EU11, EUCP1, and HV1.

In some embodiments of any one of the methods or compositions provided herein, the methods of producing rebaudioside R7-5 described herein further comprises a step of producing rebaudioside R5-1. In some embodiments, Rebaudioside R5-1 can be produced using any one of the methods of producing rebaudioside R5-1 described herein. In some embodiments, Rebaudioside R5-1 can be produced by incubating rebaudioside A with a glucansucrase and a substrate selected from the group consisting of sucrose, UDP, UDP-glucose, and combinations thereof. In some embodiments of any one of the methods or compositions provided herein, the glucansucrase used to produce rebaudioside R5-1 from rebaudioside A is IX14 or IX14M.

In some embodiments, any one of the methods of producing rebaudioside R7-5 described herein further comprises isolating the produced rebaudioside R7-5.

### Precursor Synthesis

As previously stated steviol glycosides are the chemical compounds responsible for the sweet taste of the leaves of the South American plant Stevia rebaudiana (Asteraceae) and in the plant Rubus chingii (Rosaceae). These compounds are glycosylated diterpenes. Specifically, their molecules can be viewed as a steviol molecule, with its hydroxyl hydrogen atom replaced by a glucose molecule to form an ester, and a hydroxyl hydrogen with combinations of glucose and rhamnose to form an acetal.

One method of making the compounds of interest in the current invention is to take common or inexpensive precursors, such as steviol, stevioside, Reb E, Reb D or rubusoside, such as derived chemically or produced via biosynthesis in engineered microbes, such as bacteria and/or yeast, and to synthesize target steviol glycosides, such as through known or inexpensive methods, such as Reb A.

Aspects of the present invention relate to methods involving recombinantly expressing enzymes in a microbial system capable of producing steviol. In general, such enzymes may include: a copalyl diphosphate synthase (CPS), a kaurene synthase (KS) and a geranylgeranyl diphosphate to synthase (GGPPS) enzyme. Preferably, in some embodiments, this occurs in a microbial strain that expresses an endogenous isoprenoid synthesis pathway, such as the non-mevalonate (MEP) pathway or the mevalonic acid pathway (MVA). In some embodiments of any one of the methods or compositions provided herein, the cell is a bacterial cell, such as E. coli, or a yeast cell, such as a Saccharomyces cell, Pichia cell, or a Yarrowia cell. In some embodiments of any one of the methods or compositions provided herein, the cell is an algal cell or a plant cell.

Thereafter, the precursor can be recovered from the fermentation culture and used in chemical synthesis. Typically, this is steviol though it can be kaurene, or a steviol glycoside from the cell culture. In some embodiments of any one of the methods or compositions provided herein, the steviol, kaurene and/or steviol glycosides is recovered from the gas phase while in other embodiments, an organic layer or polymeric resin is added to the cell culture, and the kaurene, steviol and/or steviol glycosides is recovered from the organic layer or polymeric resin. In some embodiments of any one of the methods or compositions provided herein, the steviol glycoside is selected from rebaudioside A. It should also be appreciated that in some embodiments, at least one enzymatic step, such as one or more glycosylation steps, are performed ex vivo.

As described herein, the enzymes used in the methods described herein have UDP-glycosyltransferase or glucansucrase activities and are useful for developing biosynthetic methods for preparing steviol glycosides that are either not present in nature or typically of low abundance in natural sources, such as rebaudioside R5-1, R6-5, R6-6, and R7-5, respectively.

The substrate can be any natural or synthetic compound capable of being converted into a steviol glycoside compound in a reaction catalyzed by one or more UDP-glucosyltransferases or glucansucrases. For example, the substrate can be natural stevia extract, steviol, steviol-13-O-glucoside, steviol-19-O-glucoside, 2-bioside, rubusoside, stevioside, rebaudioside A, rebaudioside D, rebaudioside D3, rebaudioside Z1, rebaudioside Z2, or rebaudioside E. The substrate can be a pure compound or a mixture of different compounds.

Also described herein is a coupling reaction system in which the enzymes (e.g., UDP transferases) described herein can function in combination with one or more additional enzymes (e.g., sucrose synthase) to improve the efficiency or modify the outcome of the overall biosynthesis of steviol glycoside compounds. For example, the additional enzyme may regenerate the UDP-glucose needed for the glycosylation reaction by converting the UDP produced from the glycosylation reaction back to UDP-glucose (using, for example, sucrose as a donor of the glucose residue), thus improving the efficiency of the glycosylation reaction.

Sucrose synthase catalyzes the chemical reaction between UDP-glucose and D-fructose to produce UDP and sucrose. Sucrose synthase is a glycosyltransferase. The systematic name of this enzyme class is UDP-glucose:D-fructose 2-alpha-D-glucosyltransferase. Other names in common use include UDP glucose-fructose glucosyltransferase, sucrose synthetase, sucrose-UDP glucosyltransferase, sucrose-uridine diphosphate glucosyltransferase, and uridine diphosphoglucose-fructose glucosyltransferase. Addition of the sucrose synthase to the reaction mixture that includes a uridine diphospho glycosyltransferase creates a “UGT-SUS coupling system”. In the UGT-SUS coupling system, UDP-glucose can be regenerated from UDP and sucrose, which allows for omitting the addition of extra UDP-glucose to the reaction mixture or using UDP in the reaction mixture.

Suitable sucrose synthase for use in the methods described herein include Arabidopsis sucrose synthase I, an Arabidopsis sucrose synthase 3 and a Vigna radiate sucrose synthase. In some embodiments of any one of the methods or compositions provided herein, the sucrose synthase or sucrose synthase domain is an Arabidopsis thaliana sucrose synthase I.

Suitable UDP-glycosyltransferase includes any UGT known in the art as capable of catalyzing one or more reactions in the biosynthesis of steviol glycoside compounds, such as UGT85C2, UGT74G1, HV1, UGT76G1, or the functional homologs thereof. In some embodiments, the UDP-glycotransferase used in any one of the methods described herein is UGT76G1. In some embodiments, the UDP-glycotransferase used in any one of the methods described herein is a UGT76G1-sucrose synthase fusion enzyme. In some embodiments, the UDP-glycotransferase used in any one of the methods described herein is HV1 UGT. In some embodiments, the UDP-glycotransferase used in any one of the methods described herein is a HV1 UGT-sucrose synthase fusion enzyme. In some embodiments, the UDP-glycotransferase used in any one of the methods described herein is a EU11 UGT. In some embodiments, the UDP-glycotransferase used in any one of the methods described herein is a EU11 UGT-sucrose synthase fusion enzyme. In some embodiments, the UDP-glycotransferase used in any one of the methods described herein is a EUCP1 UGT. In some embodiments, the UDP-glycotransferase used in any one of the methods described herein is a EUCP1-sucrose synthase fusion enzyme. In some embodiments, the UDP-glycotransferase used in any one of the methods described herein is a UGT76G1 L200A variant (LA). In some embodiments, the UDP-glycotransferase used in any one of the methods described herein is a LA-sucrose synthase fusion enzyme. In some embodiments, the UDP-glycotransferase used in any one of the methods described herein is a UGT76G1 CP1 variant. In some embodiments, the UDP-glycotransferase used in any one of the methods described herein is a CP1-sucrose synthase fusion enzyme.

Suitable glucansucrases includes any glucansucrase known in the art as capable of catalyzing one or more reactions in the biosynthesis of steviol glycoside compounds, such as Gtf180-ΔN glucansucrase, or the functional homolog thereof. In some embodiments, the glucansucrase used in any one of the methods described herein is a Gtf180-ΔN glucansucrase. In some embodiments, the glucansucrase used in any one of the methods described herein is a Gtf180-ΔN glucansucrase-Q1140E.

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by Greene Publishing and Wiley-Interscience, 1987; (the entirety of each of which is hereby incorporated herein by reference).

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described herein.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

Glycosylation is often considered a ubiquitous reaction controlling the bioactivity and storage of plant natural products. Glycosylation of small molecules is catalyzed by a superfamily of transferases in most plant species that have been studied to date. These glycosyltransferases (GTs) have been classified into over 60 families. Of these, the family 1 GT enzymes, also known as the UDP glycosyltransferases (UGTs) and UDP-rhamnosyltransferase, transfer sugar moieties to specific acceptor molecules. These are the molecules that transfer such sugar moieties in the steviol glycosides to help create various rebaudiosides. Each of these enzymes have their own activity profile and preferred structure locations where they transfer their activated sugar moieties.

### Reaction Mixtures, Nucleic Acids and Cellular Systems

In some embodiments of any one of the methods provided, the reaction mixture is in vitro, i.e., the method described herein is performed in vitro. For in vitro reactions, isolated enzymes (e.g., UDP-glycosyltransferase, the sucrose synthase, and/or the UDP-glycosyltransferase fusion enzymes) can be added to the in vitro reaction mixture.

In some embodiments of any one of the methods provided, the reaction mixture is a cell-based reaction mixture, i.e., the reaction is performed in a cell. For cell-based reactions, the enzymes (e.g., UDP-glycosyltransferase, the sucrose synthase, and/or the UDP-glycosyltransferase fusion enzymes) are expressed in a host cell.

In some embodiments of any one of the methods provided, the enzymes (e.g., UDP-glycosyltransferase, the sucrose synthase, and/or the UDP-glycosyltransferase fusion enzymes) are expressed from nucleotide sequences encoding them, respectively. As such, nucleic acids encoding any one of the enzymes described herein are provided. The present disclosure further provides host cells comprising a nucleotide sequence having at least 80% (e.g., at least 80%, at least 85%, at least 90%, at least 95%, at least 96%, at least 97%, at least 98%, at least 99% or 100%) identity to any one of SEQ ID NOs: 4, 6, 8, 10, 12, 14, 16, 18, 20, 22, 24, 26, and 30.

In some embodiments of any one of the methods provided, the enzymes (e.g., glucansucrase) are expressed from nucleotide sequences encoding them. As such, nucleic acids encoding any one of the enzymes described herein are provided. The present disclosure further provides host cells comprising a nucleotide sequence having at least 80% (e.g., at least 80%, at least 85%, at least 90%, at least 95%, at least 96%, at least 97%, at least 98%, at least 99% or 100%) identity to any one of SEQ ID NOs: 2 and 16.

In some embodiments of any one of the methods provided, the host cell is selected from the group consisting of a yeast, a non-steviol glycoside producing plant, an alga, a fungus, and a bacterium.

In some embodiments of any one of the methods provided, the host cell is selected from the group consisting of Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveromyces; Candida; Hansenula; Debaryomyces; Mucor; Pichia; Torulopsis; Aspergillus; Arthrobotrys; Brevibacteria; Microbacterium; Arthrobacter; Citrobacter; Klebsiella; Pantoea; and Clostridium. In some embodiments of any one of the methods provided, the host cell is a bacterial cell (e.g., an E. coli cell). In some embodiments of any one of the methods provided, the host cell is a yeast cell (e.g., a Saccharomyces cerevisiae cell).

In some embodiments of any one of the methods provided, the host cell is a cell isolated from plants selected from the group consisting of soybean; rapeseed; sunflower; cotton; corn; tobacco; alfalfa; wheat; barley; oats; sorghum; rice; broccoli; cauliflower; cabbage; parsnips; melons; carrots; celery; parsley; tomatoes; potatoes; strawberries; peanuts; grapes; grass seed crops; sugar beets; sugar cane; beans; peas; rye; flax; hardwood trees; softwood trees; forage grasses; Arabidopsis thaliana; rice (Oryza sativa); Hordeum yulgare; switchgrass (Panicum vigratum); Brachypodium spp.; Brassica spp.; and Crambe abyssinica.

Expression of proteins in prokaryotes is most often carried out in a bacterial host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and, 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

In an embodiment, the expression vector includes those genetic elements for expression of the recombinant polypeptide in bacterial cells. The elements for transcription and translation in the bacterial cell can include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR).

Several molecular biology techniques can be developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities and fill in recessed 3′-ends with their polymerizing activities, thereby generating blunt ended DNA segments. The blunt-ended segments are then incubated with a large molar excess of linker molecules in the presence of an enzyme that can catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In an embodiment, to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared using PCR using appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into plant or microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector,

In some embodiments, the transformed cell is an animal cell, an insect cell, a plant cell, an algal cell, a fungal cell, or a yeast cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell expression systems and expression vectors containing regulatory sequences that direct high-level expression of foreign proteins are well known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically, the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. In some embodiments, it is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYCI, HIS3, GALI, GALIO, ADHI, PGK, PH05, GAPDH, ADCI, TRPI, URA3, LEU2, ENO, TPI (useful for expression in Saccharomyces); AOXI (useful for expression in Pichia); and lac, trp, JPL, IPR, T7, tac, and trc (useful for expression in Escherichia coli).

Termination control regions may also be derived from various genes native to the microbial hosts. A termination site optionally may be included for the microbial hosts described herein.

In plant cells, the expression vectors of the subject technology can include a coding region operably linked to promoters capable of directing expression of the recombinant polypeptide of the subject technology in the desired tissues at the desired stage of development. For reasons of convenience, the polynucleotides to be expressed may comprise promoter sequences and translation leader sequences derived from the same polynucleotide. 3′ non-coding sequences encoding transcription termination signals should also be present. The expression vectors may also comprise one or more introns to facilitate polynucleotide expression.

For plant host cells, any combination of any promoter and any terminator capable of inducing expression of a coding region may be used in the vector sequences of the subject technology. Some suitable examples of promoters and terminators include those from nopaline synthase (nos), octopine synthase (ocs) and cauliflower mosaic virus (CaMV) genes. One type of efficient plant promoter that may be used is a high-level plant promoter. Such promoters, in operable linkage with an expression vector of the subject technology should be capable of promoting the expression of the vector. High level plant promoters that may be used in the subject technology include the promoter of the small subunit (ss) of the ribulose-1, 5-bisphosphate carboxylase for example from soybean (Berry-Lowe et al., J. MOLECULAR AND APP. GEN., 1:483 498 (1982), the entirety of which is hereby incorporated herein to the extent it is consistent herewith), and the promoter of the chlorophyll a/b binding protein. These two promoters are known to be light-induced in plant cells (see, for example, GENETIC ENGINEERING OF PLANTS, AN AGRICULTURAL PERSPECTIVE, A. Cashmore, Plenum, N.Y. (1983), pages 29-38; Coruzzi, G. et al., THE JOURNAL OF BIOLOGICAL CHEMISTRY, 258: 1399 (1983), and Dunsmuir, P. et al., JOURNAL OF MOLEC. APPL. GEN., 2:285 (1983), each of which is hereby incorporated herein by reference to the extent they are consistent herewith).

### EXAMPLES

### Example 1: Production of Steviol Glycoside Rebaudioside R5-1 by Enzymatic Bioconversion

In this study, full length DNA fragments of all candidate enzyme genes were commercially synthesized. Almost all codons of the cDNA were changed to those preferred for E. coli (Genscript, NJ). The synthesized UGT DNA was cloned into a bacterial expression vector pETite N-His SUMO Kan Vector (Lucigen) and the synthesized glucansucrase DNA was cloned into pET15b vector.

Each expression construct was transformed into E. coli BL21 (DE3), which was subsequently grown in TB media containing 50 μg/mL kanamycin or carbenicillin at 37° C. until reaching an OD600 of 0.8-1.0. Protein expression was induced by addition of 0.5-1 mM isopropyl β-D-1-thiogalactopyranoside (IPTG) and the culture was further grown at 16° C. for 22 hr. Cells were harvested by centrifugation (3,000×g; 10 min; 4° C.). The cell pellets were collected and were either used immediately or stored at −80° C.

The cell pellets typically were re-suspended in lysis buffer (50 mM potassium phosphate buffer or Tris-HCl buffer, pH 7.2, 25 μg/ml lysozyme, 5 μg/ml DNase I, 20 mM imidazole, 500 mM NaCl, 10% glycerol, and 0.4% Triton X-100). The cells were disrupted by sonication under 4° C., and the cell debris was clarified by centrifugation (18,000×g; 30 min). Supernatant was loaded to an equilibrated (equilibration buffer: 50 mM potassium phosphate buffer or Tris-HCl buffer, pH 7.2, 20 mM imidazole, 500 mM NaCl, 10% glycerol) Ni-NTA (Qiagen) affinity column. After loading of protein sample, the column was washed with equilibration buffer to remove unbound contaminant proteins. The His-tagged recombinant polypeptides were eluted by equilibration buffer containing 250 mM imidazole.

The purified candidate UGTs recombinant polypeptides were assayed for glycosylation activity by using various steviol glycosides as substrate. Typically, the recombinant polypeptide was tested in a 200 μl in vitro reaction system. The reaction system contained 50 mM potassium phosphate buffer, pH 7.2, 3 mM MgCl₂, steviol glycoside substrate, UDP-glucose or UDP and/or sucrose synthase (SUS). The reaction was performed at 30-37° C. and 50 ul reaction was terminated by adding 200 μL 1-butanol at various time points. The samples were extracted three times with 200 μL 1-butanol. The pooled fraction was dried and dissolved in 100 μL 80% methanol for high-performance liquid chromatography (HPLC) analysis.

The purified glucansucrase candidates were assayed for α-glycosylation activity by using rebaudioside A as substrate. The reaction system contained 25 mM Acetate buffer (pH 4.8), 1 mM CaCl₂), 80 g/L rebaudioside A, 0.3 mM sucrose and 0.2 g/L glucansucrase enzyme. The reaction was performed at 37° C. and 50 ul reaction was terminated by adding 200 μL 1-butanol at various time points. The samples were extracted three times with 200 μL 1-butanol. The pooled fraction was dried and dissolved in 100 μL 80% methanol for high-performance liquid chromatography (HPLC) analysis.

HPLC analysis was then performed using a Dionex UPLC ultimate 3000 system (Sunnyvale, CA), including a quaternary pump, a temperature-controlled column compartment, an auto sampler and a UV absorbance detector. A Synergi Hydro-RP column (Phenomenex) with guard column was used for the characterization of steviol glycosides in the pooled samples. Acetonitrile in water was used for isocratic elution in the HPLC analysis. The detection wavelength used in the HPLC analysis was 210 nm.

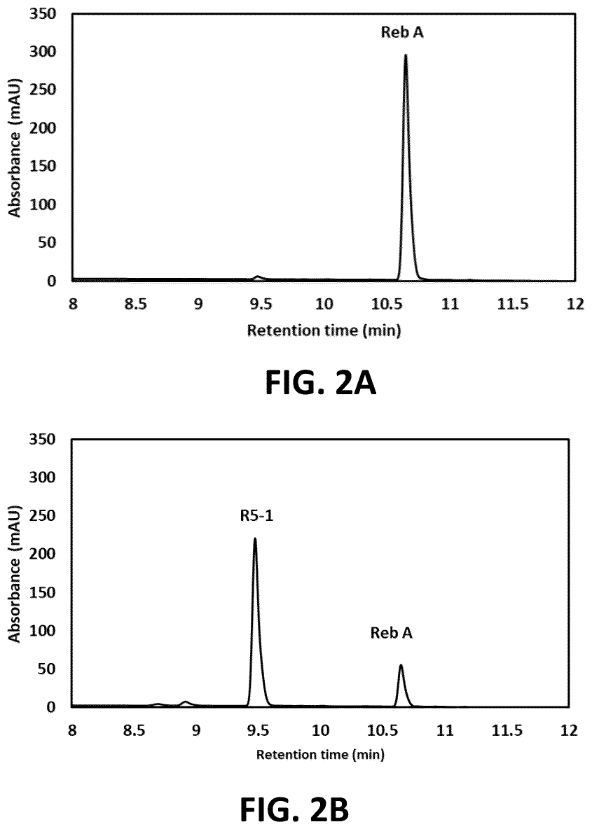

The wild-type Gtf180-ΔN glucansucrase enzyme (IX14, SEQ ID NO: 15) from Lactobacillus reuteri 180 was found to catalyze the α-glucosylation of the steviol glycoside rebaudioside A, using sucrose as glucosyl donor in a transglucosylation process. In particular, its Q1140E-mutant (IX14M, SEQ ID NO: 1), efficiently α-glucosylated rebaudioside A (Reb A) to produce rebaudioside R5-1 with the formation of an α-1,6 glucosyl linkage at C19 residue of rebaudioside A. In this invention, we cloned codon optimized Q1140E mutant into pET15b E. coli expression vector and transformed the expression construct into BL21(DE3). The engineered strain was induced by IPTG and the recombinant enzyme (IX14M) was purified by Ni NTA resin and tested the enzymatic activity using rebaudioside A and sucrose substrate. After optimization of reaction condition, we can enhance the bioconversion rate to produce large amount of rebaudioside R5-1 compound. As shown in FIG. 2A-2B, IX14M enzyme can transfer glucose moiety from sucrose, sugar donor, to rebaudioside A (sugar acceptor) and form rebaudioside R5-1 steviol glycoside compound. The majority of Reb A can be converted to R5-1 at 3 hr in the reaction (FIG. 2B). The produced rebaudioside R5-1 was extracted by butanol and dried by vacuum centrifugation. The extracted rebaudioside R5-1 can be used for further UGT enzymatic assay.

### Example 2: Identification of Rebaudioside R5-1 Production Via LC-MS Analysis

In order to confirm the produced compound, the produced compound was analyzed by LC-MS analysis comparing to standards and its identity confirmed.

The same sample from the above enzymatic bioconversion was analyzed by LC-MS using the Synergy Hydro-RP column. Mobile phase A was 0.1% formic acid in water, and mobile phase B was 0.1% formic acid in acetonitrile. The flow rate was 0.6 ml/minute. Mass spectrometry analysis of the samples was done on the Q Exactive Hybrid Quadrupole-Orbitrap Mass Spectrometer (Thermo Fisher Scientific) with an optimized method in positive ion mode.

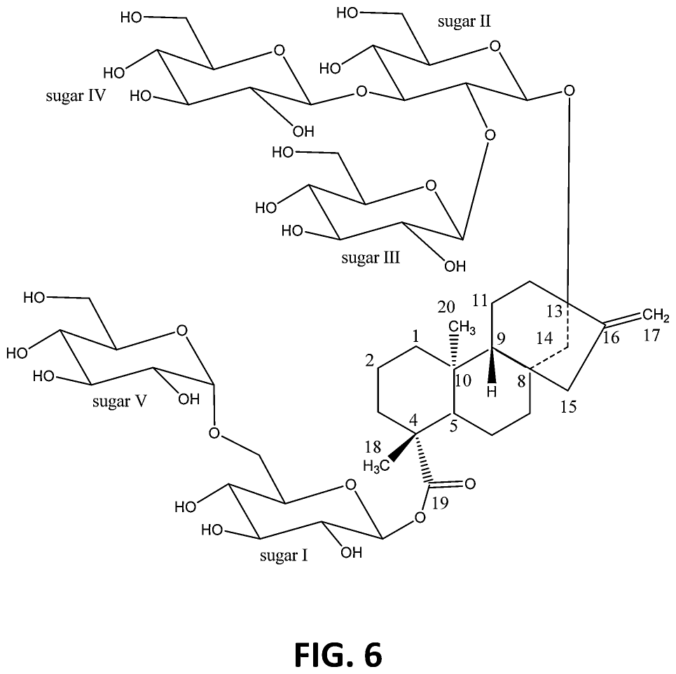

The molecular formula of compound rebaudioside R5-1 has been deduced as C₅₀H₈₀O₂₈ on the basis of its positive high resolution (HR) mass spectrum which showed an [M-H]-adduct ion at m/z 1127.4767 (calculated 1127.4758). The predicted structure of rebaudioside R5-1 is presented in FIG. 6.

### Example 3: Structure of Rebaudioside R5-1 as Analyzed by NMR

The produced rebaudioside R5-1 compound was purified by semi preparative chromatography as described above.

High resolution mass spectral data were generated with a LTQ Orbitrap Discovery HRESIMS instrument, with its resolution set to 70 k. Scanned data from m/z 100 to 1500 in positive ion electrospray mode. The needle voltage was set to 4 kV; the other source conditions were sheath gas=50, aux gas=10, sweep gas=2 (all gas flows in arbitrary units), capillary voltage=30V, capillary temperature=300° C., and tube lens voltage=75. Sample was dissolved and diluted with 50% methanol and injected 5 microliters.

NMR spectra were acquired on Bruker Avance DRX 500 MHz instrument, TMS as internal standard, using standard pulse sequences. The 1D (¹H and ¹³C) and 2D (COSY, TOCSY, HMQC, and HMBC) NMR spectra were performed in C₅D₅N.

The ¹H NMR spectrum of rebaudioside R5-1 showed the presence of two methyl singlets at δ 1.29 and 1.31, two olefinic protons as singlets at δ 4.98 and 5.60 of an exocyclic double bond, nine methylene and two methine protons between δ 0.72-2.64, characteristic for the ent-kaurane diterpenoids isolated earlier from the genus Stevia. The basic skeleton of ent-kaurane diterpenoids was supported by TOCSY (H-1/H-2; H-2/H-3; H-5/H-6; H-6/H-7; H-9/H-11; H-11/H-12) and HMBC (H-1/C-2, C-10; H-3/C-1, C-2, C-4, C-5, C-18, C-19; H-5/C-4, C-6, C-7, C-9, C-10, C-18, C-19, C-20; H-9/C-8, C-10, C-11, C-12, C-14, C-15; H-14/C-8, C-9, C-13, C-15, C-16 and H-17/C-13, C-15, C-16) correlations. The ¹H NMR spectrum of R5-1 also showed the presence of anomeric protons resonating at δ 5.06 (d, J=9.5 Hz), 5.31 (d, J=9.5 Hz), 5.53 (d, J-9.5 Hz), 6.01 (d. J=8.5 Hz), and 5.36 (d. J=4.5 Hz), and suggested five sugar units in its structure, the first four with β-orientation, and last one with α-orientation, as reported for steviol glycosides.

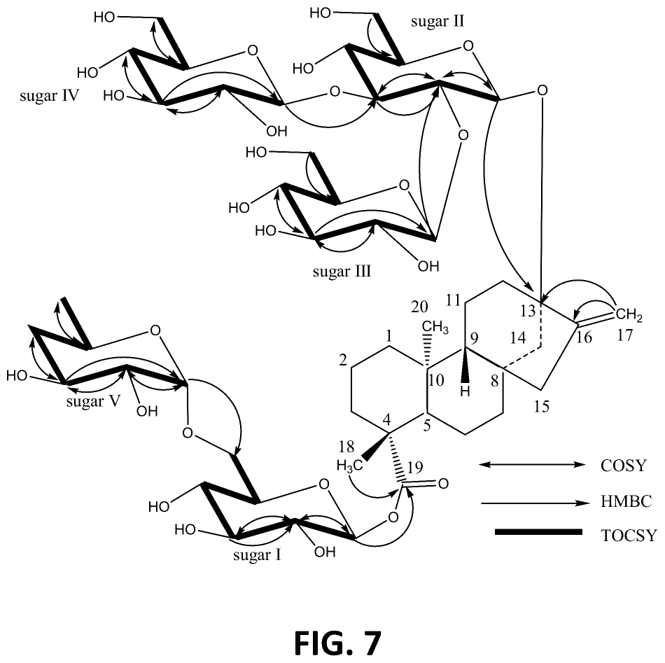

A comparison of the ¹H and ¹³C NMR spectrum of rebaudioside R5-1 with Reb A suggested that compound R5-1 is also a steviol glycoside which has three glucose residues that are attached at the C-13 hydroxyl as a 2,3-branched glucotriosyl substituent and 1-substituted glucobiosyl moiety in the form of an ester at C-19 leaving the assignment of the additional glucosyl moiety. The key COSY and HMBC correlations (FIG. 7) suggested the placement of the fifth glucosyl moiety at C-6 position of Sugar I. The ¹H and ¹³C NMR values for selected protons and carbons in R5-1 were assigned on the basis of HSQC, COSY, TOCSY, and HMBC correlations (Table 1).

Based on the results of NMR and mass spectral studies and in comparison with the spectral values of rebaudioside A-G1 reported from the literature, the structure of rebaudioside R5-1 was assigned as 13-[(2-O-β-D-glucopyranosyl-3-O-β-D-glucopyranosyl-β-D-glucopyranosyl)oxy] ent-kaur-16-en-19-oic acid-[(6-O-α-D-glucopyranosyl-β-D-glucopyranosyl)ester].

TABLE 1
¹H and ¹³C NMR spectral data (chemical shifts and
coupling constants) for rebaudioside R5-1 ^{a-c}.

| Position | ¹H NMR | ¹³C NMR |
|---|---|---|
| 1 | 0.72 (t, 11.0 Hz), 1.75 (d, 11.0 Hz) | 40.8 |
| 2 | 1.39 (br d, 9.5 Hz), 2.14 (br d, 12.5 Hz) | 19.4 |
| 3 | 0.97 (m), 2.35 (d, 12.5 Hz) | 38.4 |
| 4 | | 44.0 |
| 5 | 0.99 (d, 12.0 Hz) | 57.2 |
| 6 | 1.87 (m), 2.26 (m) | 22.3 |
| 7 | 1.28 (m), 1.35 (m) | 41.75 |
| 8 | | 42.3 |
| 9 | 0.86 (d, 7.5 Hz) | 54.1 |
| 10 | | 41.0 |
| 11 | 1.69 (m) | 20.6 |
| 12 | 1.94 (m), 2.27 (m) | 37.3 |
| 13 | | 86.8 |
| 14 | 1.77 (m), 2.64 (d, 14.5 Hz) | 44.3 |
| 15 | 2.03 (br s) | 47.7 |
| 16 | | 154.5 |
| 17 | 4.98 (s), 5.60 (s) | 104.2 |
| 18 | 1.28 (s) | 28.4 |
| 19 | | 177.4 |
| 20 | 1.31 (s) | 15.8 |
| 1′ | 6.01 (d, 8.5 Hz) | 95.5 |
| 2′ | 4.08 (m) | 73.8 |
| 3′ | 3.96 (m) | 77.1 |
| 4′ | 4.14 (m) | 71.6 |
| 5′ | 4.07 (m) | 78.8 |
| 6′ | 4.47 (m), 4.21 (m) | 67.9 |
| 1″ | 5.06 (d, 9.5 Hz) | 98.3 |
| 2″ | 4.34 (m) | 80.9 |
| 3″ | 4.18 (m) | 88.0 |
| 4″ | 3.85 (m) | 70.6 |
| 5″ | 3.76 (m) | 77.5 |
| 6″ | 4.36 (m), 4.29 (m) | 62.5 |
| 1′″ | 5.53 (d, 9.5 Hz) | 104.7 |
| 2′″ | 4.16 (m) | 76.3 |
| 3′″ | 4.23 (m) | 78.3 |
| 4′″ | 4.24 (m) | 71.9 |
| 5′″ | 3.89 (m) | 78.5 |
| 6′″ | 4.44 (m), 4.20 (m) | 62.8 |
| 1″″ | 5.31 (d, 9.5 Hz) | 103.9 |
| 2″″ | 4.01 (m) | 75.2 |
| 3″″ | 3.95 (m) | 77.2 |
| 4″″ | 3.86 (m) | 70.8 |
| 5″″ | 4.16 (m) | 76.4 |
| 6″″ | 4.43 (m), 4.35 (m) | 62.8 |
| 1′″″ | 5.36 (d, 4.5 Hz) | 99.6 |
| 2′″″ | 4.09 (m) | 74.0 |
| 3′″″ | 4.55 (m) | 75.4 |
| 4′″″ | 4.66 (m) | 72.0 |
| 5′″″ | 4.45 (m) | 74.0 |
| 6′″″ | 4.28 (m), 4.54 (m) | 62.5 |

^{a} recorded in pyridine-d5 at 299 K; ^{b} assignments made on the basis of COSY, TOCSY, HSQC and HMBC correlations; Chemical shift values are in δ (ppm).

### Example 4: Production of Novel Steviol Glycoside Rebaudioside R6-5 by Enzymatic Bioconversion

According to the current invention, full length DNA fragments of all candidate enzyme genes were commercially synthesized. Almost all codons of the cDNA were changed to those preferred for E. coli (Genscript, NJ). The synthesized UGT DNA was cloned into a bacterial expression vector pETite N-His SUMO Kan Vector (Lucigen).

Each expression construct was transformed into E. coli BL21 (DE3), which was subsequently grown in TB media containing 50 μg/mL kanamycin or carbenicillin at 37° C. until reaching an OD600 of 0.8-1.0. Protein expression was induced by addition of 0.5-1 mM isopropyl β-D-1-thiogalactopyranoside (IPTG) and the culture was further grown at 16° C. for 22 hr. Cells were harvested by centrifugation (3,000×g; 10 min; 4° C.). The cell pellets were collected and were either used immediately or stored at −80° C.

The cell pellets typically were re-suspended in lysis buffer (50 mM potassium phosphate buffer or Tris-HCl buffer, pH 7.2, 25 μg/ml lysozyme, 5 μg/ml DNase I, 20 mM imidazole, 500 mM NaCl, 10% glycerol, and 0.4% Triton X-100). The cells were disrupted by sonication under 4° C., and the cell debris was clarified by centrifugation (18,000×g; 30 min). Supernatant was loaded to an equilibrated (equilibration buffer: 50 mM potassium phosphate buffer or Tris-HCl buffer, pH 7.2, 20 mM imidazole, 500 mM NaCl, 10% glycerol) Ni-NTA (Qiagen) affinity column. After loading of protein sample, the column was washed with equilibration buffer to remove unbound contaminant proteins. The His-tagged recombinant polypeptides were eluted by equilibration buffer containing 250 mM imidazole. The purified candidate UGTs recombinant polypeptides were assayed for glycosylation activity by using various steviol glycosides as substrate. Typically, the recombinant polypeptide was tested in a 200 μl in vitro reaction system. The reaction system contained 50 mM potassium phosphate buffer, pH 7.2, 3 mM MgCl₂, steviol glycoside substrate, UDP-glucose or UDP and/or sucrose synthase (SUS). The reaction was performed at 30-37° C. and 50 ul reaction was terminated by adding 200 μL 1-butanol at various time points. The samples were extracted three times with 200 μL 1-butanol. The pooled fraction was dried and dissolved in 100 μL 80% methanol for high-performance liquid chromatography (HPLC) analysis.

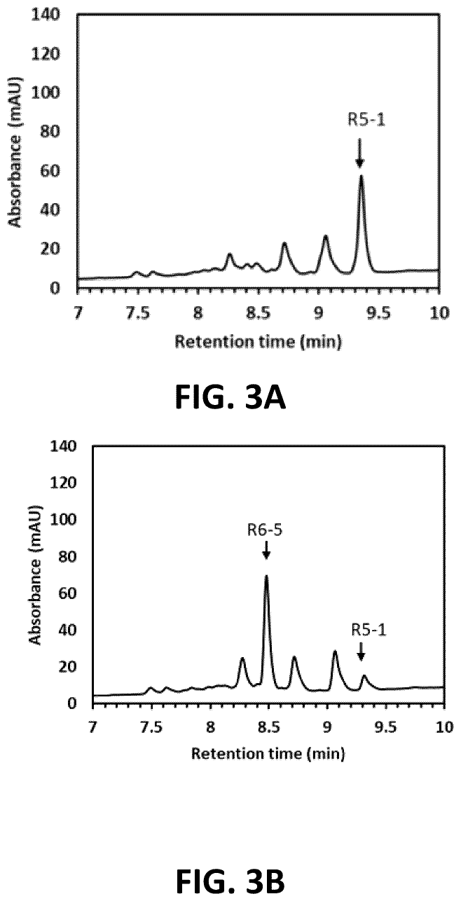

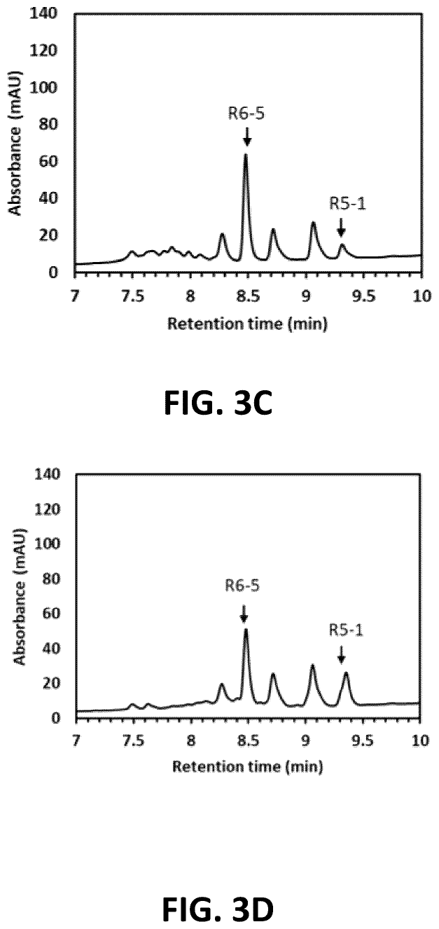

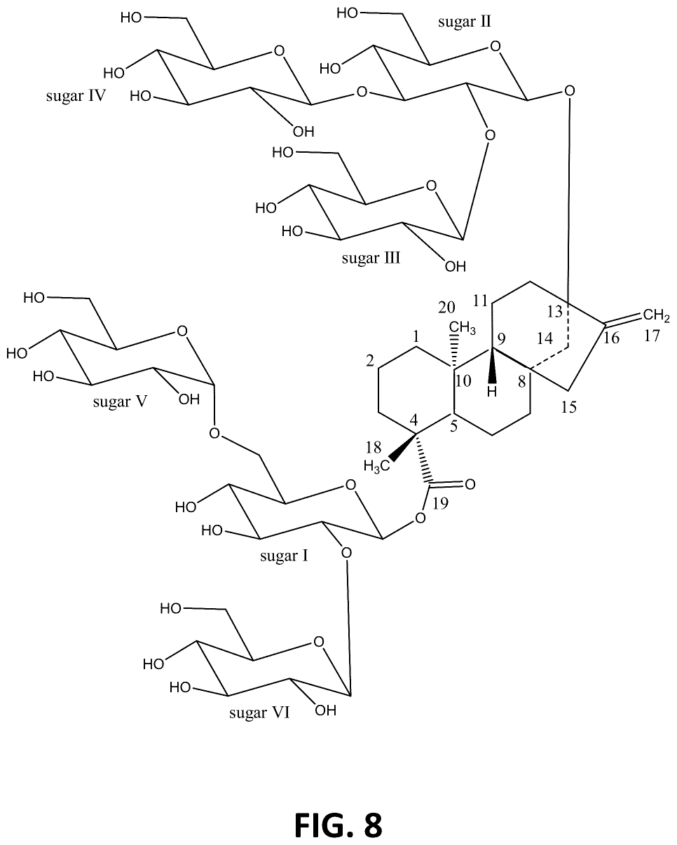

The purified candidate UGT recombinant polypeptides were assayed for β 1,2 glycosylation activity using rebaudioside R5-1 as the substrate. As shown in FIGS. 3A-D, all selected UGT candidates can convert rebaudioside R5-1 compound to a novel steviol glycoside (rebaudioside R6-5). Rebaudioside R6-5 can be formed by glycosylation of the C-2′ of the C-19-O-glucose of R5-1 by EU11 (SEQ ID NO: 3), EUCP1 (SEQ ID NO: 5) and HV1 (SEQ ID NO: 7) UGTs. The structure of rebaudioside R6-5 (FIG. 8) was identified by NMR analysis as described in Example 2.

### Example 5: Identification of Rebaudioside R6-5 Production Via LC-MS Analysis

In order to confirm the produced compound, the produced compound was analyzed by LC-MS analysis comparing to standards and its identity confirmed.

The same sample from the above enzymatic bioconversion was analyzed by LC-MS using the Synergy Hydro-RP column. Mobile phase A was 0.1% formic acid in water, and mobile phase B was 0.1% formic acid in acetonitrile. The flow rate was 0.6 ml/minute. Mass spectrometry analysis of the samples was done on the Q Exactive Hybrid Quadrupole-Orbitrap Mass Spectrometer (Thermo Fisher Scientific) with an optimized method in positive ion mode.

The molecular formula of compound rebaudioside R6-5 has been deduced as C₅₆H₉₀O₃₃ on the basis of its positive high resolution (HR) mass spectrum which showed an [M-H]⁻ adduct ion at m/z 1289.5313 (calculated 1289.5286). The predicted structure of rebaudioside R6-5 is presented in FIG. 8.

### Example 6: Structure of Rebaudioside R6-5 as Analyzed by NMR

The produced R6-5 compound was purified by semi preparative chromatography as described above.

High resolution mass spectral data were generated with a LTQ Orbitrap Discovery HRESIMS instrument, with its resolution set to 70 k. Scanned data from m/z 100 to 1500 in positive ion electrospray mode. The needle voltage was set to 4 kV; the other source conditions were sheath gas=50, aux gas=10, sweep gas=2 (all gas flows in arbitrary units), capillary voltage=30V, capillary temperature=300° C., and tube lens voltage=75. Sample was dissolved and diluted with 50% methanol and injected 5 microliters.

NMR spectra were acquired on Bruker Avance DRX 500 MHZ instrument, TMS as internal standard, using standard pulse sequences. The 1D (¹H and ¹³C) and 2D (COSY, TOCSY, HMQC, and HMBC) NMR spectra were performed in C₅D₅N.

The ¹H NMR spectrum of rebaudioside R6-5 showed the presence of two methyl singlets at δ 1.31 and 1.41, two olefinic protons as singlets at δ 4.97 and 5.60 of an exocyclic double bond, nine methylene and two methine protons between δ 0.74-2.75, characteristic for the ent-kaurane diterpenoids isolated earlier from the genus Stevia. The basic skeleton of ent-kaurane diterpenoids was supported by COSY/TOCSY (H-1/H-2; H-2/H-3; H-5/H-6; H-6/H-7; H-9/H-11; H-11/H-12) and HMBC (H-1/C-2, C-10; H-3/C-1, C-2, C-4, C-5, C-18, C-19; H-5/C-4, C-6, C-7, C-9, C-10, C-18, C-19, C-20; H-9/C-8, C-10, C-11, C-12, C-14, C-15; H-14/C-8, C-9, C-13, C-15, C-16 and H-17/C-13, C-15, C-16) correlations. The ¹H NMR spectrum of R6-5 also showed the presence of anomeric protons resonating at δ 6.23 (d, J=7.0 Hz), 5.22 (d, J=7.5 Hz), 5.58 (d, J-8.0 Hz), 5.43 (d. J=7.5 Hz), and 5.41 (d. J=7.5 Hz), and suggested five sugar units in its structure with β-orientation; and an anomeric protons resonating at δ 5.35 (d. J=4.5 Hz), one sugar units in its structure with α-orientation, as reported for steviol glycosides.

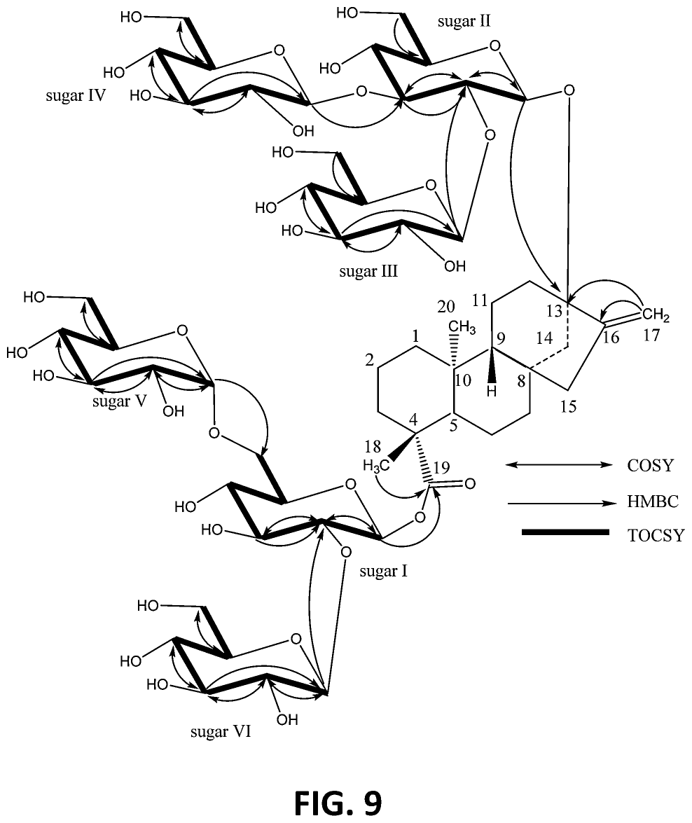

A comparison of the ¹H and ¹³C NMR spectrum of rebaudioside R6-5 with Reb A suggested that compound rebaudioside R6-5 is also a steviol glycoside which has three glucose residues that are attached at the C-13 hydroxyl as a 2,3-branched glucotriosyl substituent and a 2, 6-branched glucobiosyl substituent moiety in the form of an ester at C-19 leaving the assignment of the additional glucosyl moiety (FIG. 8). The key COSY/TOCSY and HMBC correlations (FIG. 9) suggested the placement of the fifth glucosyl moiety at C-6 position and the sixth glucosyl moiety at C-2 position of Sugar I. The ¹H and ¹³C NMR values for selected protons and carbons in R6-5 were assigned on the basis of HSQC, COSY, TOCSY, and HMBC correlations (Table 2).

TABLE 2
¹H and ¹³C NMR spectral data (chemical shifts and
coupling constants) for rebaudioside R6-5^{a-c}.

| Position | ¹H NMR | ¹³C NMR |
|---|---|---|
| 1 | 0.74 (t, 11.0 Hz), 1.84 (m) | 40.7 |
| 2 | 1.44 (m), 2.21 (m) | 19.9 |
| 3 | 1.08 (br t, 13.0), 2.76 (br t, 12.5 Hz) | 37.7 |
| 4 | | 44.3 |
| 5 | 1.01 (d, 12.0 Hz) | 57.4 |
| 6 | 1.89 (m), 2.19 (m) | 22.2 |
| 7 | 1.27 (br t, 10.5 Hz), 1.38 (br s) | 41.8 |
| 8 | | 42.5 |
| 9 | 0.89 (d, 7.5 Hz) | 54.0 |
| 10 | | 40.7 |
| 11 | 1.66 (br s), 1.78 (br s) | 20.5 |
| 12 | 1.97 (m), 2.41(m) | 37.8 |
| 13 | | 87.0 |
| 14 | 1.83 (d, 10.5 Hz), 2.59 (d, 10.5 Hz) | 43.6 |
| 15 | 2.04 (br s) | 47.7 |
| 16 | | 154.4 |
| 17 | 4.97 (s), 5.60 (s) | 104.2 |
| 18 | 1.41 (s) | 29.1 |
| 19 | | 175.8 |
| 20 | 1.31 (s) | 16.8 |
| 1′ | 6.23 (d, 7.0 Hz) | 93.2 |
| 2′ | 4.27 (m) | 80.8 |
| 3′ | 4.18 (m) | 78.6 |
| 4′ | 4.08 (m) | 73.9 |
| 5′ | 4.01 (m) | 77.1 |
| 6′ | 4.44 (m), 4.26 (m) | 68.6 |
| 1″ | 5.22(d,7.5 Hz) | 98.1 |
| 2″ | 4.37 (m) | 80.8 |
| 3″ | 4.62 (m) | 86.9 |
| 4″ | 4.08 (m) | 69.7 |
| 5″ | 3.7 (m) | 77.2 |
| 6″ | 4.24 (m), 4.12 (m) | 62.1 |
| 1′″ | 5.58 (d, 8.0 Hz) | 104.4 |
| 2′″ | 4.12 (m) | 76.4 |
| 3′″ | 4.22 (m) | 78.2 |
| 4′″ | 4.2 (m) | 71.9 |
| 5′″ | 3.88 (m) | 78.5 |
| 6′″ | 4.39 (m), 4.44 (m) | 62.9 |
| 1″″ | 5.43 (d, 7.5 Hz) | 104.3 |
| 2″″ | 4.02 (m) | 75.3 |
| 3″″ | 3.98 (m) | 78.5 |
| 4″″ | 4.01 (m) | 71.1 |
| 5″″ | 4.04 (m) | 76.1 |
| 6″″ | 4.42 (m), 4.37 (m) | 62.6 |
| 1′″″ | 5.35 (d, 4.5 Hz) | 100.7 |
| 2′″″ | 4.07 (m) | 73.9 |
| 3′″″ | 4.5 (m) | 75.3 |
| 4′″″ | 4.05 (m) | 71.8 |
| 5′″″ | 4.38 (m) | 73.8 |
| 6′″″ | 4.57 (m), 4.38 (m) | 62.8 |
| 1″″″ | 5.41 (d, 7.5 Hz) | 105.7 |
| 2″″″ | 4.05 (m) | 76.2 |
| 3″″″ | 4.23 (m) | 78.2 |
| 4″″″ | 4.08 (m) | 73.9 |
| 5″″″ | 3.88 (m) | 78.1 |
| 6″″″ | 4.20 (m), 4.24 (m) | 64.8 |

^{a}recorded in pyridine-d5 at 299 K; ^{b}assignments made on the basis of COSY, TOCSY, HSQC and HMBC correlations; Chemical shift values are in δ (ppm).

Based on the results of NMR and mass spectral studies and in comparison with the spectral values of rebaudioside M2 reported from the literature, there is orientation difference between anomeric proton of the sixth sugar units, Rebaudioside M2 is β-orientation, however, compound rebaudioside R6-5 is α-orientation, the structure was assigned as 13-[(2-O-β-D-glucopyranosyl-3-O-β-D-glucopyranosyl-β-D-glucopyranosyl)oxy] ent-kaur-16-en-19-oic acid-[(2-O-β-D-glucopyranosyl-6-O-α-D-glucopyranosyl-β-D-gluco-pyranosyl)ester].

### Example 7: Production of Novel Steviol Glycoside Rebaudioside R6-6 by Enzymatic Bioconversion

According to the current invention, full length DNA fragments of all candidate enzyme genes were commercially synthesized. Almost all codons of the cDNA were changed to those preferred for E. coli (Genscript, NJ). The synthesized UGT DNA was cloned into a bacterial expression vector pETite N-His SUMO Kan Vector (Lucigen).

Each expression construct was transformed into E. coli BL21 (DE3), which was subsequently grown in TB media containing 50 μg/mL kanamycin or carbenicillin at 37° C. until reaching an OD600 of 0.8-1.0. Protein expression was induced by addition of 0.5-1 mM isopropyl β-D-1-thiogalactopyranoside (IPTG) and the culture was further grown at 16° C. for 22 hr. Cells were harvested by centrifugation (3,000×g; 10 min; 4° C.). The cell pellets were collected and were either used immediately or stored at −80° C.

The cell pellets typically were re-suspended in lysis buffer (50 mM potassium phosphate buffer or Tris-HCl buffer, pH 7.2, 25 μg/ml lysozyme, 5 μg/ml DNase I, 20 mM imidazole, 500 mM NaCl, 10% glycerol, and 0.4% Triton X-100). The cells were disrupted by sonication under 4° C., and the cell debris was clarified by centrifugation (18,000×g; 30 min). Supernatant was loaded to an equilibrated (equilibration buffer: 50 mM potassium phosphate buffer or Tris-HCl buffer, pH 7.2, 20 mM imidazole, 500 mM NaCl, 10% glycerol) Ni-NTA (Qiagen) affinity column. After loading of protein sample, the column was washed with equilibration buffer to remove unbound contaminant proteins. The His-tagged recombinant polypeptides were eluted by equilibration buffer containing 250 mM imidazole.

The purified candidate UGTs recombinant polypeptides were assayed for glycosylation activity by using various steviol glycosides as substrate. Typically, the recombinant polypeptide was tested in a 200 μl in vitro reaction system. The reaction system contained 50 mM potassium phosphate buffer, pH 7.2, 3 mM MgCl₂, steviol glycoside substrate, UDP-glucose or UDP and/or sucrose synthase (SUS). The reaction was performed at 30-37° C. and 50 ul reaction was terminated by adding 200 μL 1-butanol at various time points. The samples were extracted three times with 200 μL 1-butanol. The pooled fraction was dried and dissolved in 100 μL 80% methanol for high-performance liquid chromatography (HPLC) analysis.

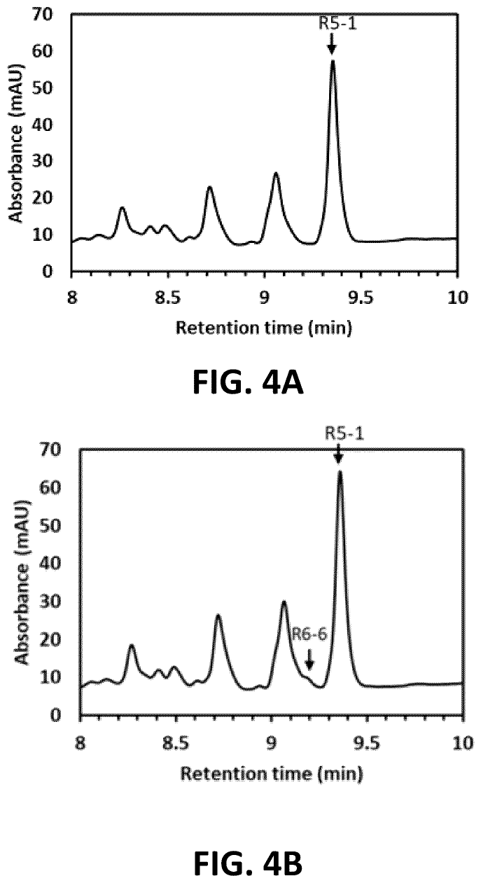

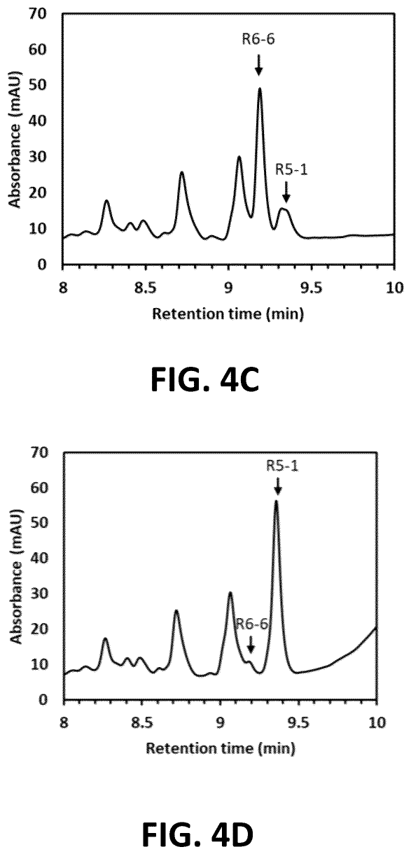

The purified candidate UGT recombinant polypeptides were assayed for β 1,3 glycosylation activity using rebaudioside R5-1 as the substrate. As shown in FIGS. 4A-D, all selected UGT candidates can convert R5-1 compound to a novel steviol glycoside (rebaudioside R6-6). Rebaudioside R6-6 can be formed by glycosylation of the C-3′ of the C-19-O-glucose of R5-1 by UGT76G1 (SEQ ID NO: 9), LA (SEQ ID NO: 11) and CP1 (SEQ ID NO: 13) UGTs. LA enzyme shows the highest activity for rebaudioside R6-6 production.

### Example 8: Identification of Rebaudioside R6-6 Production Via LC-MS Analysis

In order to confirm the produced compound, the produced compound was analyzed by LC-MS analysis comparing to standards and its identity confirmed.

The same sample from the above enzymatic bioconversion was analyzed by LC-MS using the Synergy Hydro-RP column. Mobile phase A was 0.1% formic acid in water, and mobile phase B was 0.1% formic acid in acetonitrile. The flow rate was 0.6 ml/minute. Mass spectrometry analysis of the samples was done on the Q Exactive Hybrid Quadrupole-Orbitrap Mass Spectrometer (Thermo Fisher Scientific) with an optimized method in positive ion mode.

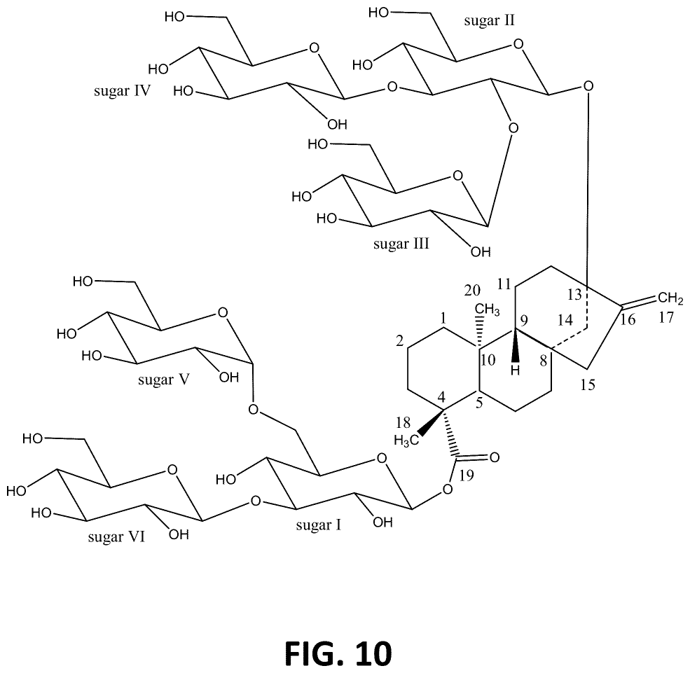

The molecular formula of compound rebaudioside R6-6 has been deduced as C₅₆H₉₀O₃₃ on the basis of its positive high resolution (HR) mass spectrum which showed an [M-H]⁻ ion at m/z 1289.5304 (calculated 1289.5286). The predicted structure of rebaudioside R6-6 is presented in FIG. 10.

### Example 9: Structure of Rebaudioside R6-6 as Analyzed by NMR

The produced rebaudioside R6-6 compound was purified by semi preparative chromatography as described above.

High resolution mass spectral data were generated with a LTQ Orbitrap Discovery HRESIMS instrument, with its resolution set to 70 k. Scanned data from m/z 100 to 1500 in positive ion electrospray mode. The needle voltage was set to 4 kV; the other source conditions were sheath gas=50, aux gas=10, sweep gas=2 (all gas flows in arbitrary units), capillary voltage=30V, capillary temperature=300° C., and tube lens voltage=75. Sample was dissolved and diluted with 50% methanol and injected 5 microliters.

NMR spectra were acquired on Bruker Avance DRX 500 MHZ instrument, TMS as internal standard, using standard pulse sequences. The 1D (¹H and ¹³C) and 2D (COSY, TOCSY, HMQC, and HMBC) NMR spectra were performed in C₅D₅N.

The ¹H NMR spectrum of rebaudioside R6-6 showed the presence of two methyl singlets at δ 1.26 and 1.29, two olefinic protons as singlets at δ 5.01 and 5.64 of an exocyclic double bond, nine methylene and two methine protons between δ 0.72-2.55, characteristic for the ent-kaurane diterpenoids isolated earlier from the genus Stevia. The basic skeleton of ent-kaurane diterpenoids was supported by COSY/TOCSY (H-1/H-2; H-2/H-3; H-5/H-6; H-6/H-7; H-9/H-11; H-11/H-12) and HMBC (H-1/C-2, C-10; H-3/C-1, C-2, C-4, C-5, C-18, C-19; H-5/C-4, C-6, C-7, C-9, C-10, C-18, C-19, C-20; H-9/C-8, C-10, C-11, C-12, C-14, C-15; H-14/C-8, C-9, C-13, C-15, C-16 and H-17/C-13, C-15, C-16) correlations. The ¹H NMR spectrum of R6-6 also showed the presence of anomeric protons resonating at δ 6.00 (d, J=8.0 Hz), 5.51 (d, J=7.5 Hz), 5.32 (d, J=8.0 Hz), 5.28 (d, J=3.5 Hz), and 5.18 (d, J=8.0 Hz), suggested five sugar units in its structure with β-orientation; and an anomeric protons resonating at δ 5.28 (d, J=3.5 Hz), one sugar units in its structure with α-orientation (FIG. 10), as reported for steviol glycosides.

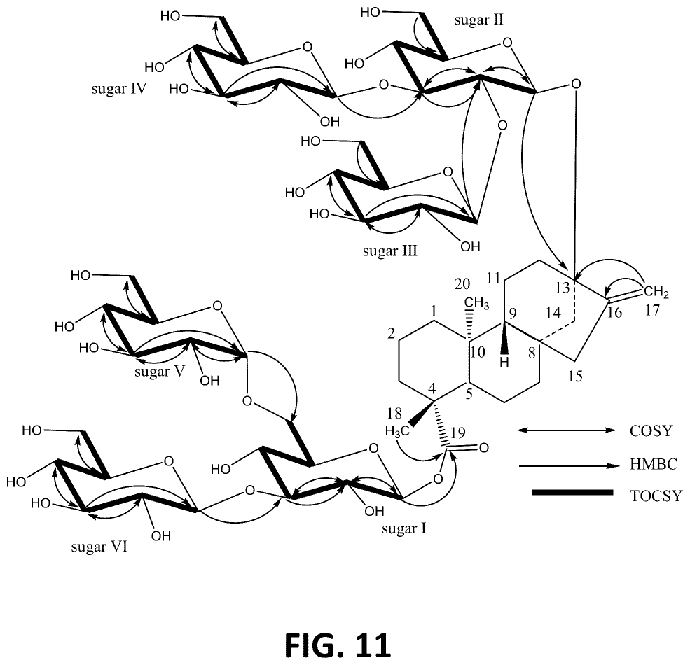

A comparison of the ¹H and ¹³C NMR spectrum of rebaudioside R6-6 with rebaudioside A suggested that compound R6-6 is also a steviol glycoside which has three glucose residues that are attached at the C-13 hydroxyl as a 2,3-branched glucotriosyl substituent and a 3, 6-branched glucobiosyl substituent moiety in the form of an ester at C-19 leaving the assignment of the additional glucosyl moiety. The key COSY/TOCSY and HMBC correlations (FIG. 11) suggested the placement of the fifth glucosyl moiety at C-6 position and the sixth glucosyl moiety at C-3 position of sugar I. The ¹H and ¹³C NMR values for selected protons and carbons in R6-6 were assigned on the basis of HSQC, COSY, TOCSY, and HMBC correlations (Table 3).

Based on the results of NMR and mass spectral studies and comparison with the spectral values of R6-5, there is difference between the placement of the sixth glucosyl moiety at position of sugar I. The structure was assigned as 13-[(2-O-β-D-glucopyranosyl-3-O-β-D-glucopyranosyl-β-D-glucopyranosyl)oxy] ent-kaur-16-en-19-oic acid-[(3-O-β-D-glucopyranosyl-6-O-α-D-glucopyranosyl-β-D-gluco-pyranosyl)ester].

TABLE 3
¹H and ¹³C NMR spectral data (chemical shifts and
coupling constants) for rebaudioside R6-6^{a-c}.

| Position | ¹H NMR | ¹³C NMR |
|---|---|---|
| 1 | 0.72 (t, 12.5/10.5 Hz), 1.75 (d, 11.0 Hz) | 40.8 |
| 2 | 1.39 (br d, 9.5 Hz), 2.14 (br d, 12.5 Hz) | 19.4 |
| 3 | 1.01 (br t, 12.0 Hz), 2.35 (d, 12.5 Hz) | 38.4 |
| 4 | | 44.0 |
| 5 | 0.99 (d, 12.0 Hz) | 57.2 |
| 6 | 1.87 (m), 2.26 (m) | 22.3 |
| 7 | 1.28 (br s) | 41.7 |
| 8 | | 42.3 |
| 9 | 0.86 (d, 7.5 Hz) | 54.1 |
| 10 | | 40.7 |
| 11 | 1.69 (m) | 20.6 |
| 12 | 1.94 (m), 2.27 (m) | 37.3 |
| 13 | | 86.8 |
| 14 | 1.77 (m), 2.55 (m) | 44.3 |
| 15 | 2.03 (br s) | 47.7 |
| 16 | | 154.0 |
| 17 | 5.01 (s), 5.64 (s) | 104.7 |
| 18 | 1.29 (s) | 28.4 |
| 19 | | 176.9 |
| 20 | 1.26 (s) | 15.7 |
| 1′ | 6.00 (d, 8.0 Hz) | 94.99 |
| 2′ | 3.92 (m) | 76.60 |
| 3′ | 4.14 (m) | 88.9 |
| 4′ | 4.08 (m) | 73.99 |
| 5′ | 4.23 (m) | 78.4 |
| 6′ | 4.34 (m), 4.08 (m) | 66.99 |
| 1″ | 5.02 (d, 7.5 Hz) | 97.93 |
| 2″ | 4.28 (m) | 80.5 |
| 3″ | 4.18 (m) | 87.5 |
| 4″ | 4.05 (m) | 69.48 |
| 5″ | 3.74 (m) | 77.49 |
| 6″ | 4.14 (m), 4.21 (m) | 62.47 |
| 1′″ | 5.51 (d, 7.5 Hz) | 104.47 |
| 2′″ | 3.91 (m) | 76.6 |
| 3′″ | 4.23 (m) | 78.42 |
| 4′″ | 4.16 (m) | 71.9 |
| 5′″ | 3.93 (m) | 78.2 |
| 6′″ | 4.21 (m), 4.54 (m) | 62.3 |
| 1″″ | 5.32 (d, 8.0 Hz) | 104.57 |
| 2″″ | 3.97 (m) | 75.35 |
| 3″″ | 4.06 (m) | 78.51 |
| 4″″ | 4.00 (m) | 71.52 |
| 5″″ | 4.12 (m) | 76.17 |
| 6″″ | 4.34 (m), 4.48 (m) | 62.56 |
| 1′″″ | 5.28 (d, 3.5 Hz) | 100.07 |
| 2′″″ | 4.06 (m) | 73.98 |
| 3′″″ | 4.58 (m) | 75.3 |
| 4′″″ | 4.24 (m) | 71.9 |
| 5′″″ | 4.42 (m) | 73.7 |
| 6′″″ | 4.34 (m), 4.38 (m) | 63.09 |
| 1″″″ | 5.18 (d, 8.0 Hz) | 104.72 |
| 2″″″ | 3.98 (m) | 75.3 |
| 3″″″ | 4.19 (m) | 78.25 |
| 4″″″ | 4.41 (m) | 73.7 |
| 5″″″ | 4.07 (m) | 78.5 |
| 6″″″ | 4.19 (m), 4.23 (m) | 64.8 |

^{a} recorded in pyridine-d5 at 299 K; ^{b}assignments made on the basis of COSY, TOCSY, HSQC and HMBC correlations; Chemical shift values are in δ (ppm).

### Example 10: Production of Steviol Glycoside Rebaudioside R7-5 by Enzymatic Bioconversion

According to the current invention, full length DNA fragments of all candidate enzyme genes were commercially synthesized. Almost all codons of the cDNA were changed to those preferred for E. coli (Genscript, NJ). The synthesized UGT DNA was cloned into a bacterial expression vector pETite N-His SUMO Kan Vector (Lucigen).

Each expression construct was transformed into E. coli BL21 (DE3), which was subsequently grown in TB media containing 50 μg/mL kanamycin or carbenicillin at 37° C. until reaching an OD600 of 0.8-1.0. Protein expression was induced by addition of 0.5-1 mM isopropyl β-D-1-thiogalactopyranoside (IPTG) and the culture was further grown at 16° C. for 22 hr. Cells were harvested by centrifugation (3,000×g; 10 min; 4° C.). The cell pellets were collected and were either used immediately or stored at −80° C.

The cell pellets typically were re-suspended in lysis buffer (50 mM potassium phosphate buffer or Tris-HCl buffer, pH 7.2, 25 μg/ml lysozyme, 5 μg/ml DNase I, 20 mM imidazole, 500 mM NaCl, 10% glycerol, and 0.4% Triton X-100). The cells were disrupted by sonication under 4° C., and the cell debris was clarified by centrifugation (18,000×g; 30 min). Supernatant was loaded to an equilibrated (equilibration buffer: 50 mM potassium phosphate buffer or Tris-HCl buffer, pH 7.2, 20 mM imidazole, 500 mM NaCl, 10% glycerol) Ni-NTA (Qiagen) affinity column. After loading of protein sample, the column was washed with equilibration buffer to remove unbound contaminant proteins. The His-tagged recombinant polypeptides were eluted by equilibration buffer containing 250 mM imidazole.

The purified candidate UGTs recombinant polypeptides were assayed for glycosylation activity by using various steviol glycosides as substrate. Typically, the recombinant polypeptide was tested in a 200 μl in vitro reaction system. The reaction system contained 50 mM potassium phosphate buffer, pH 7.2, 3 mM MgCl₂, steviol glycoside substrate, UDP-glucose or UDP and/or sucrose synthase (SUS). The reaction was performed at 30-37° C. and 50 ul reaction was terminated by adding 200 μL 1-butanol at various time points. The samples were extracted three times with 200 μL 1-butanol. The pooled fraction was dried and dissolved in 100 μL 80% methanol for high-performance liquid chromatography (HPLC) analysis.

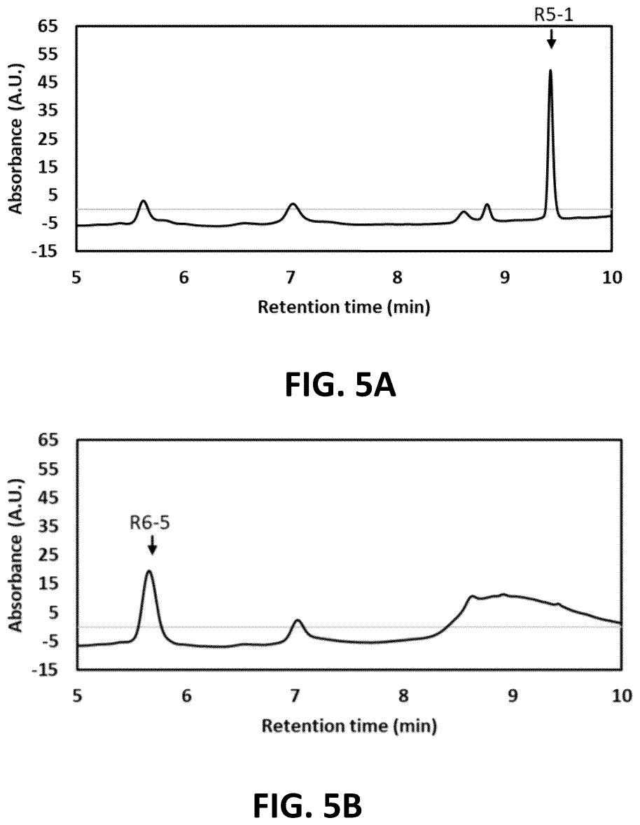

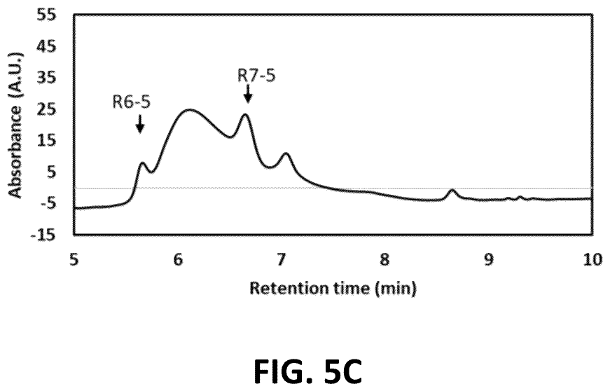

The purified candidate UGT recombinant polypeptides were assayed in combination. The combination of IX14M, EUCP1 and LA enzymes using Reb A as substrate yielded the novel steviol glycoside rebaudioside R7-5. In the reaction, IX14M, EUCP1 and LA enzyme was added in the reaction sequentially. Reb A can be converted to rebaudioside R5-1 by the IX14M enzyme (FIG. 5A). The produced rebaudioside R5-1 can be converted to rebaudioside R6-5 (FIG. 5B) after EUCP1 enzyme addition. The produced rebaudioside R6-5 can be converted to a novel steviol glycoside (rebaudioside R7-5) (FIG. 5C) after LA enzyme addition. Rebaudioside R7-5 is a novel steviol glycoside containing 6 glucosyl moieties.

### Example 11: Identification of Rebaudioside R7-5 Production Via LC-MS Analysis

In order to confirm the produced compound, the produced compound was analyzed by LC-MS analysis comparing to standards and its identity confirmed.

The same sample from the above enzymatic bioconversion was analyzed by LC-MS using the Synergy Hydro-RP column. Mobile phase A was 0.1% formic acid in water, and mobile phase B was 0.1% formic acid in acetonitrile. The flow rate was 0.6 ml/minute. Mass spectrometry analysis of the samples was done on the Q Exactive Hybrid Quadrupole-Orbitrap Mass Spectrometer (Thermo Fisher Scientific) with an optimized method in positive ion mode.

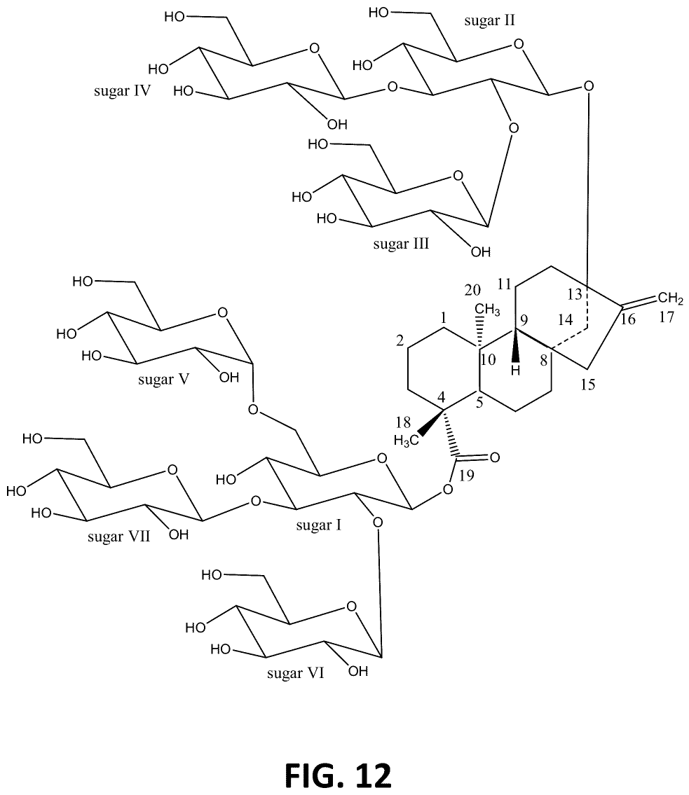

The molecular formula of compound rebaudioside R7-5 has been deduced as C₆₂H₉₉O₃₈ on the basis of its negative high resolution (HR) mass spectrum which showed an [M-H]⁻ adduct ion at m/z 1451.5840 (calculated 1451.5814). The predicted structure of rebaudioside R7-5 is presented in FIG. 12.

### Example 12: Structure of Rebaudioside R7-5 as Analyzed by NMR

The produced rebaudioside R7-5 compound was purified by semi preparative chromatography as described above.

High resolution mass spectral data were generated with a LTQ Orbitrap Discovery HRESIMS instrument, with its resolution set to 70 k. Scanned data from m/z 100 to 1500 in positive ion electrospray mode. The needle voltage was set to 4 kV; the other source conditions were sheath gas=50, aux gas=10, sweep gas=2 (all gas flows in arbitrary units), capillary voltage=30V, capillary temperature=300° C., and tube lens voltage=75. Sample was dissolved and diluted with 50% methanol and injected 5 microliters.

NMR spectra were acquired on Bruker Avance DRX 500 MHz instrument, TMS as internal standard, using standard pulse sequences. The 1D (¹H and ¹³C) and 2D (COSY, TOCSY, HMQC, and HMBC) NMR spectra were performed in C₅D₅N.

The ¹H NMR spectrum of rebaudioside R7-5 showed the presence of two methyl singlets at δ 1.28 and 1.38, two olefinic protons as singlets at δ 4.91 and 5.70 of an exocyclic double bond, nine methylene and two methine protons between δ 0.75-2.74, characteristic for the ent-kaurane diterpenoids isolated earlier from the genus Stevia. The basic skeleton of ent-kaurane diterpenoids was supported by COSY/TOCSY (H-1/H-2; H-2/H-3; H-5/H-6; H-6/H-7; H-9/H-11; H-11/H-12) and HMBC (H-1/C-2, C-10; H-3/C-1, C-2, C-4, C-5, C-18, C-19; H-5/C-4, C-6, C-7, C-9, C-10, C-18, C-19, C-20; H-9/C-8, C-10, C-11, C-12, C-14, C-15; H-14/C-8, C-9, C-13, C-15, C-16 and H-17/C-13, C-15, C-16) correlations. The ¹H NMR spectrum of R7-5 also showed the presence of anomeric protons resonating at δ 6.27 (d. J=8.5 Hz), 5.61 (d, J=8.0 Hz), 5.56 (d. J=7.5 Hz), 5.50 (d. J=7.5 Hz), 5.22 (d. J=3.5 Hz), and 5.12 (d, J=8.0 Hz), and suggested six sugar units in its structure with β-orientation; and an anomeric protons resonating at δ 5.22 (d. J=3.5 Hz), one sugar units in its structure with α-orientation (FIG. 12), as reported for steviol glycosides.

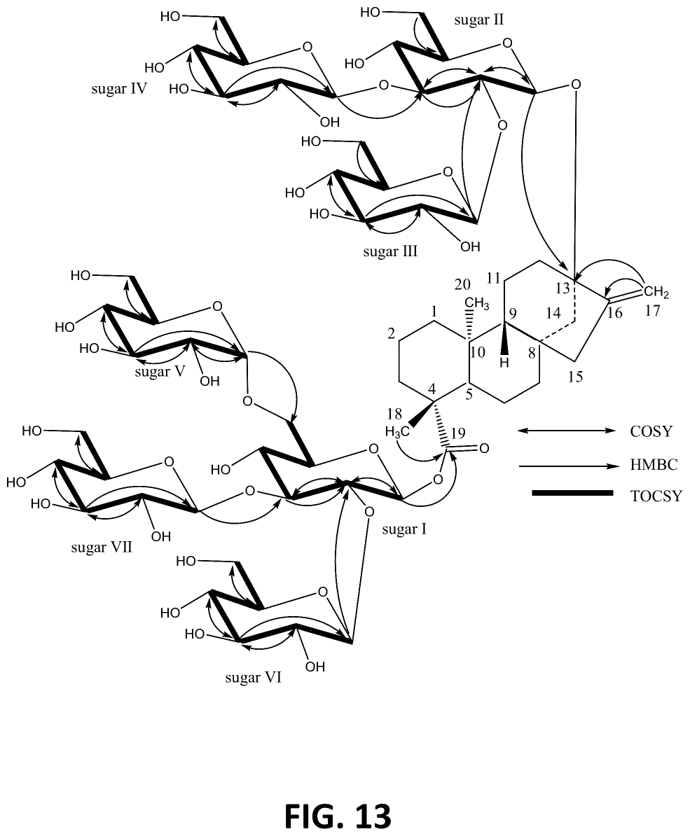

A comparison of the ¹H and ¹³C NMR spectrum of rebaudioside R7-5 with rebaudioside A suggested that compound rebaudioside R7-5 is also a steviol glycoside which has three glucose residues that are attached at the C-13 hydroxyl as a 2,3-branched glucotriosyl substituent and a 2, 3, 6-branched glucobiosyl substituent moiety in the form of an ester at C-19 leaving the assignment of the additional glucosyl moiety. The key COSY/TOCSY and HMBC correlations (FIG. 13) suggested the placement of the fifth glucosyl moiety at C-6 position the sixth glucosyl moiety at C-2 position, and the seventh glucosyl moiety at C-3 position of Sugar I. The ¹H and ¹³C NMR values for selected protons and carbons in rebaudioside R7-5 were assigned on the basis of HSQC, COSY, TOCSY, and HMBC correlations (Table 4).

Based on the results of NMR and mass spectral studies and comparison with the spectral values of compounds rebaudioside R6-5 and rebaudioside R6-6, there is one more glucopyranosyl in compound rebaudioside R7-5. The structure was assigned as 13-[(2-O-β-D-glucopyranosyl-3-O-β-D-glucopyranosyl-β-D-glucopyranosyl)oxy] ent-kaur-16-en-19-oic acid-[(2-O-β-D-glucopyranosyl-3-O-β-D-glucopyranosyl-6-O-α-D-glucopyranosyl-β-D-gluco-pyranosyl)ester].

TABLE 4
¹H and ¹³C NMR spectral data (chemical shifts and
coupling constants) for rebaudioside R7-5^{a-c}.

| Position | ¹H NMR | ¹³C NMR |
|---|---|---|
| 1 | 1.78 (m), 0.75 (td, 13.5, 3.5Hz) | 40.36 |
| 2 | 1.33 (m), 2.24 (m) | 19.7 |
| 3 | 0.98 (td, 14.0, 3.0 Hz), 2.76 (br t, 11.0 Hz) | 38.47 |
| 4 | | 44.46 |
| 5 | 1.04 (d, 13.5 Hz) | 57.39 |
| 6 | 2.41 (m), 2.21 (m) | 23.5 |
| 7 | 1.80, 1.41 | 42.6 |
| 8 | | 41.55 |
| 9 | 0.92 (d, 8.0 Hz) | 53.34 |
| 10 | | 40.02 |
| 11 | 1.66 (m), 1.78 (br d, 11.0 Hz) | 20.32 |
| 12 | 1.88 (d, 12.0 Hz), 2.39 (m) | 38.39 |
| 13 | | 87.8 |
| 14 | 2.01 (m), 2.74 (m) | 43.4 |
| 15 | 2.03, 1.90 | 46.7 |
| 16 | | 153.7 |
| 17 | 4.91 (s), 5.70 (s) | 104.91 |
| 18 | 1.28 (s) | 28.11 |
| 19 | | 177.17 |
| 20 | 1.38 (s) | 16.76 |
| 1′ | 6.27 (d, 8.5 Hz) | 94.73 |
| 2′ | 4.40 (m) | 76.73 |
| 3′ | 4.99 (m) | 88.5 |
| 4′ | 4.14 (m) | 70.11 |
| 5′ | 4.20 (m) | 76.62 |
| 6′ | 4.04 (m), 4.42 (m) | 67.03 |
| 1″ | 5.50 (d, 7.5 Hz) | 96.37 |
| 2″ | 4.17 (m) | 81.52 |
| 3″ | 5.09 (m) | 87.8 |
| 4″ | 4.08 (m) | 70.28 |
| 5″ | 3.97 (m) | 77.95 |
| 6″ | 4.21 (m), 4.36 (m) | 62.39 |
| 1′″ | 5.56 (d, 7.5 Hz) | 104.47 |
| 2′″ | 4.15 (m) | 75.92 |
| 3′″ | 3.73 (m) | 77.7 |
| 4′″ | 4.14 (m) | 70.2 |
| 5′″ | 3.97 (m) | 78.14 |
| 6′″ | 4.01 (m), 4.20 (m) | 62.38 |
| 1″″ | 5.61 (d, 8.0 Hz) | 103.6 |
| 2″″ | 4.01 (m) | 75.64 |
| 3″″ | 3.98 (m) | 77.95 |
| 4″″ | 4.19 (m) | 71.54 |
| 5″″ | 4.61 (m) | 75.2 |
| 6″″ | 4.13 (m) | 62.63 |
| 1′″″ | 5.22 (d, 3.5 Hz) | 99.91 |
| 2′″″ | 4.08 (m) | 74.23 |
| 3′″″ | 4.61 (m) | 75.23 |
| 4′″″ | 4.24 (m) | 71.96 |
| 5′″″ | 4.47 (m) | 73.59 |
| 6′″″ | 4.20 (m), 4.35 (m) | 62.50 |
| 1″″″ | 5.65 (d, 7.0 Hz) | 104.37 |
| 2″″″ | 4.21 (m) | 75.24 |
| 3″″″ | 3.98 (m) | 77.95 |
| 4″″″ | 4.14 (m) | 70.11 |
| 5″″″ | 3.65 (m) | 77.85 |
| 6″″″ | 4.29 (m), 4.52 (m) | 64.08 |
| 1″″″′ | 5.12 (d, 8.0 Hz) | 103.98 |
| 2″″″′ | 3.90 (m) | 75.5 |
| 3″″″′ | 4.20 (m) | 78.36 |
| 4″″″′ | 4.18 (m) | 71.6 |
| 5″″″′ | 3.65 (m) | 77.85 |
| 6″″″′ | 4.37 (m), 4.69 (m) | 63.92 |

^{a}recorded in pyridine-d5 at 299 K; ^{b}assignments made on the basis of COSY, TOCSY, HSQC and HMBC correlations; Chemical shift values are in δ (ppm).

### REFERENCES

1. Te Poele, E. M., Devlamynck, T., Jäger, M. Gerwig G J, Van de Walle D, Dewettinck K, Hirsch A K H, Kamerling J P, Soetaert W, Dijkhuizen L. Glucansucrase (mutant) enzymes from Lactobacillus reuteri 180 efficiently transglucosylate Stevia component rebaudioside A, resulting in a superior taste. Sci Rep. 2018, 8, 1516.
2. Prakash, I.; Bunders, C.; Devkota, K. P.; Charan, R. D.; Ramirez, C.; Priedemann, C.; Markosyan, A. Isolation and Characterization of a Novel Rebaudioside M Isomer from a Bioconversion Reaction of Rebaudioside A and NMR Comparison Studies of Rebaudioside M Isolated from Stevia rebaudiana Bertoni and Stevia rebaudiana Morita. Biomolecules 2014, 4, 374-389.
3. Bedir, E., Toyang, N. J., Khan, I. A., Walker, L. A., Clark, A. M. A new dammarane type triterpene glycoside from Polyscias fulva. Journal of Natural Products. 2001; 64, 95-97.
4. Chaturvedula, V. S. P., Yu, O., Mao, G. NMR Spectral Analysis of rebaudioside A, a major sweet diterpene glycoside of Stevia rebaudiana Bertoni at various temperatures, International Journal of Pharmaceutical Science Invention, 2013, 2, 36-40.
5. Chaturvedula, V. S. P., Chen, S., Yu, O., Mao, G. Isolation, NMR spectral analysis and hydrolysis studies of a hepta pyranosyl diterpene glycoside from Stevia rebaudiana Bertoni. Biomolecules, 2013, 3, 733-740.
6. Gerwig, G. J., Te Poele E. M., Dojkhuizen L., Kamerling J. P. Structural analysis of rebaudioside A derivatives obtained by Lactobacillus reuteri 180 glucansucrase-catalyzed trans-α-glucosylation. Carbonydr Res. 2017; 440-441:51-62

Sequences:

Gtf180-ΔN-Q1140E* glucansucrase (IX14M): Amino Acid Sequence (SEQ ID NO: 1)
INGQQYYIDPTTGQPRKNFLLQNGNDWIYFDKDTGAGTNALKLQFDKGTISADEQYR
RGNEAYSYDDKSIENVNGYLTADTWYRPKQILKDGTTWTDSKETDMRPILMVWWPN
TVTQAYYLNYMKQYGNLLPASLPSFSTDADSAELNHYSELVQQNIEKRISETGSTDWL
RTLMHEFVTKNSMWNKDSENVDYGGLQLQGGFLKYVNSDLTKYANSDWRLMNRTA
TNIDGKNYGGAEFLLANDIDNSNPVVQAEELNWLYYLMNFGTITGNNPEANFDGIRV
DAVDNVDVDLLSIARDYFNAAYNMEQSDASANKHINILEDWGWDDPAYVNKIGNPQ
LTMDDRLRNAIMDTLSGAPDKNQALNKLITQSLVNRANDNTENAVIPSYNFVRAHDS
NAEDQIRQAIQAATGKPYGEFNLDDEKKGMEAYINDQNSTNKKWNLYNMPSAYTILL
TNKDSVPRVYYGDLYQDGGQYMEHKTRYFDTITNLLKTRVKYVAGGQTMSVDKNGI
LTNVRFGKGAMNATDTGTDETRTEGIGVVISNNTNLKLNDGESVVLHMGAAHKNQK
YRAVILTTEDGVKNYTNDTDAPVAYTDANGDLHFTNTNLDGQQYTAVRGYANPDVT
GYLAVWVPAGAADDQDARTAPSDEAHTTKTAYRSNAALDSNVIYEGFSNFIYWPTTE
SERTNVRIAQNADLFKSWGITTFELAPQYNSSKDGTFLDSIIDNGYAFTDRYDLGMSTP
NKYGSDEDLRNALQALHKAGLQAIADWVPDQIYNLPGKEAVTVTRSDDHGTTWEVS
PIKNVVYITNTIGGGEYQKKYGGEFLDTLQKEYPQLFSQVYPVTQTTIDPSVKIKEWSA
KYFNGTNILHRGAGYVLRSNDGKYYNLGTSTQQFLPSQLSVQDNEGYGFVKEGNNY
HYYDENKQMVKDAFIQDSVGNWYYFDKNGNMVANQSPVEISSNGASGTYLFLNNGT
SFRSGLVKTDAGTYYYDGDGRMVRNQTVSDGAMTYVLDENGKLVSESFDSSATEAH
PLKPGDLNGQK
*Q1140E refers to the mutation at amino acid 1140 in the full-length Gtf180 protein.
In SEQ ID NO: 1, Q1140E corresponds to Q399E
Gtf180-ΔN-Q1140E glucansucrase (IX14M): DNA Sequence (SEQ ID NO: 2)
ATGATCAATGGCCAGCAGTATTATATCGATCCGACGACCGGTCAGCCTCGCAAAA
ACTTTTTGCTGCAAAATGGGAACGACTGGATTTACTTTGACAAAGATACCGGGGCC
GGTACGAATGCCCTCAAACTGCAATTTGACAAGGGCACTATCAGCGCGGACGAAC
AGTACCGACGTGGTAATGAAGCGTACAGCTATGATGATAAATCTATTGAAAATGT
CAATGGGTATCTGACAGCAGATACTTGGTATCGCCCCAAGCAGATTCTGAAAGAC
GGCACCACATGGACGGATAGCAAAGAAACTGACATGCGCCCGATCTTAATGGTCT
GGTGGCCCAATACCGTGACGCAGGCCTACTATCTGAATTACATGAAACAGTATGG
AAACCTTCTGCCGGCCTCTCTGCCATCTTTTAGCACCGATGCTGATTCCGCTGAATT
AAACCATTATAGCGAGCTAGTTCAACAGAACATCGAGAAAAGAATTAGTGAAACG
GGAAGCACTGACTGGCTGCGGACCTTGATGCATGAATTTGTGACGAAAAACAGCA
TGTGGAACAAAGATTCTGAAAATGTAGATTATGGTGGCCTCCAACTTCAGGGGGG
TTTCCTGAAATACGTGAACTCCGATTTAACTAAGTACGCCAATAGCGATTGGAGAC
TGATGAACCGCACCGCGACGAACATTGATGGCAAAAATTACGGAGGTGCCGAATT
TCTTTTGGCCAACGACATTGACAATAGCAACCCAGTAGTTCAAGCAGAGGAACTG
AACTGGCTGTATTACCTCATGAACTTTGGGACCATTACCGGCAATAATCCTGAAGC
TAATTTTGATGGGATTCGAGTTGATGCGGTGGATAATGTGGACGTTGATCTTCTGT
CTATTGCTCGCGACTACTTTAATGCAGCATACAACATGGAACAATCAGATGCTTCG
GCCAACAAACATATCAACATTCTCGAAGATTGGGGCTGGGATGACCCAGCGTACG
TTAACAAGATTGGCAACCCTCAGTTAACGATGGACGACCGCCTGCGTAACGCGAT
TATGGATACGCTGTCAGGTGCGCCAGACAAAAATCAGGCGCTGAACAAGCTGATC
ACACAATCGCTGGTGAACAGAGCGAACGATAATACCGAAAACGCGGTTATTCCAT
CCTACAATTTTGTCCGCGCTCACGATAGTAATGCCGAAGATCAAATTCGACAGGCA
ATCCAGGCGGCCACAGGTAAACCGTACGGAGAGTTTAATCTCGACGACGAAAAAA
AAGGTATGGAAGCGTACATTAACGACCAAAATTCGACCAACAAGAAATGGAACCT
GTACAACATGCCTTCCGCGTACACGATTCTTCTGACCAACAAAGATAGCGTACCGA
GGGTGTATTACGGGGATTTATACCAGGATGGCGGCCAGTACATGGAGCACAAGAC
CCGCTATTTTGACACCATCACAAACCTGCTGAAAACCCGTGTAAAATATGTAGCTG
GCGGTCAGACCATGAGTGTTGATAAAAATGGCATTCTGACCAATGTTCGTTTTGGT
AAAGGAGCGATGAATGCTACGGATACCGGCACCGACGAGACACGTACGGAGGGC
ATTGGTGTTGTAATATCGAACAATACCAACTTGAAGCTTAACGATGGAGAAAGCG
TGGTACTGCACATGGGTGCGGCGCATAAAAATCAAAAATACCGTGCGGTGATCTT
GACGACGGAAGATGGAGTAAAAAATTATACGAACGATACAGACGCACCCGTGGC
CTACACCGATGCTAACGGCGATCTGCATTTCACGAATACCAATCTGGATGGCCAGC
AATATACTGCGGTACGCGGTTATGCTAACCCGGATGTGACCGGCTACCTTGCTGTT
TGGGTGCCGGCAGGGGCGGCGGATGATCAGGATGCCCGCACTGCTCCGAGCGATG
AGGCGCATACGACAAAAACTGCGTATAGATCGAATGCAGCCTTGGATTCTAACGT
TATATACGAAGGTTTTAGCAACTTTATTTATTGGCCGACTACCGAAAGCGAACGAA
CAAACGTACGCATTGCTCAAAATGCGGACCTGTTTAAAAGCTGGGGAATCACCAC
CTTTGAACTGGCCCCGCAATACAATTCATCAAAAGACGGCACCTTTCTGGATTCAA
TTATTGATAACGGCTATGCATTTACCGATCGTTACGATCTGGGGATGTCTACCCCG
AACAAATACGGCTCTGATGAGGATCTCCGCAATGCGCTACAAGCCTTACACAAGG
CCGGACTCCAAGCGATTGCTGACTGGGTGCCGGATCAGATTTATAATCTGCCAGGT
AAGGAAGCTGTGACCGTAACCCGTAGCGATGACCACGGCACCACGTGGGAAGTCT
CACCGATCAAGAACGTTGTCTACATCACGAATACCATTGGTGGCGGTGAATACCA
GAAGAAATATGGTGGCGAATTTTTGGACACCTTGCAAAAAGAATACCCGCAGCTG
TTCTCACAGGTATACCCAGTGACCCAAACCACCATCGATCCGAGCGTCAAAATAA
AAGAATGGAGTGCAAAGTATTTTAACGGCACAAATATTCTTCATCGGGGCGCAGG
GTACGTACTCCGTAGCAATGACGGCAAATACTATAATCTCGGAACCTCTACTCAGC
AGTTCTTGCCGAGCCAGCTGTCAGTTCAAGATAACGAGGGATATGGGTTTGTCAA
AGAAGGTAACAATTATCATTACTATGATGAGAATAAACAAATGGTAAAGGACGCA
TTTATCCAGGACTCTGTTGGTAATTGGTATTACTTTGACAAGAACGGTAACATGGT
GGCAAACCAGTCCCCGGTGGAAATCAGTTCAAATGGGGCGTCGGGCACGTATCTC
TTCCTGAATAACGGCACCTCCTTCCGTAGTGGCTTAGTTAAGACCGATGCAGGAAC
CTACTACTATGATGGGGACGGTCGTATGGTGCGGAATCAAACGGTTTCTGACGGT
GCCATGACCTACGTGTTGGACGAAAACGGAAAACTCGTGTCCGAATCTTTTGATA
GTTCGGCGACCGAAGCCCACCCACTTAAGCCAGGCGACCTCAACGGCCAGAAGTA
A
EU11: Amino Acid Sequence (SEQ ID NO: 3)
MDSGYSSSYAAAAGMHVVICPWLAFGHLLPCLDLAQRLASRGHRVSFVSTPRNISRLP
PVRPALAPLVAFVALPLPRVEGLPDGAESTNDVPHDRPDMVELHRRAFDGLAAPFSEF
LGTACADWVIVDVFHHWAAAAALEHKVPCAMMLLGSAHMIASIADRRLERAETESP
AAAGQGRPAAAPTFEVARMKLIRTKGSSGMSLAERFSLTLSRSSLVVGRSCVEFEPET
VPLLSTLRGKPITFLGLMPPLHEGRREDGEDATVRWLDAQPAKSVVYVALGSEVPLG
VEKVHELALGLELAGTRFLWALRKPTGVSDADLLPAGFEERTRGRGVVATRWVPQM
SILAHAAVGAFLTHCGWNSTIEGLMFGHPLIMLPIFGDQGPNARLIEAKNAGLQVARN
DGDGSFDREGVAAAIRAVAVEEESSKVFQAKAKKLQEIVADMACHERYIDGFIQQLRS
YKD
EU11: DNA Sequence (SEQ ID NO: 4)
ATGGATTCGGGTTACTCTTCCTCCTATGCGGCGGCTGCGGGTATGCACGTTGTTAT
CTGTCCGTGGCTGGCTTTTGGTCACCTGCTGCCGTGCCTGGATCTGGCACAGCGTC
TGGCTTCACGCGGCCATCGTGTCAGCTTCGTGTCTACCCCGCGCAATATTTCGCGT
CTGCCGCCGGTTCGTCCGGCACTGGCTCCGCTGGTTGCATTTGTCGCTCTGCCGCT
GCCGCGCGTGGAAGGTCTGCCGGATGGTGCGGAAAGTACCAACGACGTGCCGCAT
GATCGCCCGGACATGGTTGAACTGCACCGTCGTGCATTCGATGGTCTGGCAGCACC
GTTTTCCGAATTTCTGGGTACGGCGTGCGCCGATTGGGTGATCGTTGACGTCTTTC
ATCACTGGGCGGCGGCGGCGGCGCTGGAACATAAAGTTCCGTGTGCAATGATGCT
GCTGGGCTCAGCTCACATGATTGCGTCGATCGCAGACCGTCGCCTGGAACGTGCA
GAAACCGAAAGTCCGGCTGCGGCCGGCCAGGGTCGCCCGGCAGCTGCGCCGACCT
TCGAAGTGGCCCGCATGAAACTGATTCGTACGAAAGGCAGCTCTGGTATGAGCCT
GGCAGAACGCTTTAGTCTGACCCTGTCCCGTAGTTCCCTGGTGGTTGGTCGCAGTT
GCGTTGAATTTGAACCGGAAACCGTCCCGCTGCTGTCCACGCTGCGTGGTAAACCG
ATCACCTTTCTGGGTCTGATGCCGCCGCTGCATGAAGGCCGTCGCGAAGATGGTGA
AGACGCAACGGTGCGTTGGCTGGATGCACAGCCGGCTAAAAGCGTCGTGTATGTC
GCCCTGGGCTCTGAAGTGCCGCTGGGTGTGGAAAAAGTTCACGAACTGGCACTGG
GCCTGGAACTGGCTGGCACCCGCTTCCTGTGGGCACTGCGTAAACCGACGGGTGT
GAGCGATGCGGACCTGCTGCCGGCCGGTTTTGAAGAACGTACCCGCGGCCGTGGT
GTTGTCGCAACGCGTTGGGTCCCGCAAATGAGCATTCTGGCGCATGCCGCAGTGG
GCGCCTTTCTGACCCACTGTGGTTGGAACAGCACGATCGAAGGCCTGATGTTTGGT
CACCCGCTGATTATGCTGCCGATCTTCGGCGATCAGGGTCCGAACGCACGTCTGAT
TGAAGCGAAAAATGCCGGCCTGCAAGTTGCGCGCAACGATGGCGACGGTTCTTTC
GACCGTGAGGGTGTGGCTGCGGCCATTCGCGCAGTGGCTGTTGAAGAAGAATCAT
CGAAAGTTTTTCAGGCGAAAGCCAAAAAACTGCAAGAAATCGTCGCGGATATGGC
CTGCCACGAACGCTACATTGATGGTTTCATTCAGCAACTGCGCTCCTACAAAGACT
AA
EUCP1: Amino Acid Sequence (SEQ ID NO: 5)
MGSSGMSLAERFSLTLSRSSLVVGRSCVEFEPETVPLLSTLRGKPITFLGLMPPLHEGRR
EDGEDATVRWLDAQPAKSVVYVALGSEVPLGVEKVHELALGLELAGTRFLWALRKP
TGVSDADLLPAGFEERTRGRGVVATRWVPQMSILAHAAVGAFLTHCGWNSTIEGLMF
GHPLIMLPIFGDQGPNARLIEAKNAGLQVARNDGDGSFDREGVAAAIRAVAVEEESSK
VFQAKAKKLQEIVADMACHERYIDGFIQQLRSYKDDSGYSSSYAAAAGMHVVICPWL
AFGHLLPCLDLAQRLASRGHRVSFVSTPRNISRLPPVRPALAPLVAFVALPLPRVEGLP
DGAESTNDVPHDRPDMVELHRRAFDGLAAPFSEFLGTACADWVIVDVFHHWAAAAA
LEHKVPCAMMLLGSAHMIASIADRRLERAETESPAAAGQGRPAAAPTFEVARMKLIR
TK
EUCP1: DNA Sequence (SEQ ID NO: 6)
ATGGGTAGCTCGGGCATGTCCCTGGCGGAACGCTTTTCGCTGACGCTGAGTCGCTC
ATCCCTGGTTGTTGGTCGCAGTTGTGTTGAATTTGAACCGGAAACCGTTCCGCTGC
TGTCTACGCTGCGCGGCAAACCGATTACCTTCCTGGGTCTGATGCCGCCGCTGCAT
GAAGGCCGTCGCGAAGATGGTGAAGACGCCACGGTGCGTTGGCTGGATGCTCAGC
CGGCGAAATCGGTGGTTTATGTCGCACTGGGCAGCGAAGTGCCGCTGGGTGTCGA
AAAAGTGCACGAACTGGCCCTGGGCCTGGAACTGGCAGGCACCCGCTTTCTGTGG
GCACTGCGTAAACCGACGGGCGTTAGCGATGCTGACCTGCTGCCGGCGGGTTTCG
AAGAACGCACCCGCGGCCGTGGTGTCGTGGCCACCCGTTGGGTGCCGCAAATGTC
CATTCTGGCTCATGCGGCCGTTGGCGCATTTCTGACCCACTGCGGTTGGAACAGCA
CGATCGAAGGCCTGATGTTTGGTCATCCGCTGATTATGCTGCCGATCTTCGGCGAT
CAGGGTCCGAACGCACGCCTGATCGAAGCCAAAAATGCAGGCCTGCAAGTTGCGC
GTAACGATGGCGACGGTAGCTTTGACCGCGAAGGTGTCGCAGCTGCGATTCGTGC
TGTGGCGGTTGAAGAAGAAAGCAGCAAAGTCTTCCAGGCCAAAGCGAAAAAACT
GCAAGAAATCGTGGCTGATATGGCGTGTCATGAACGCTATATTGACGGCTTTATCC
AGCAACTGCGTTCTTACAAAGATGACAGTGGCTATAGTTCCTCATACGCCGCAGCT
GCGGGTATGCATGTTGTCATTTGCCCGTGGCTGGCGTTTGGTCACCTGCTGCCGTG
TCTGGATCTGGCACAGCGCCTGGCATCTCGCGGTCACCGTGTTTCGTTCGTCAGCA
CCCCGCGCAATATCAGTCGTCTGCCGCCGGTTCGTCCGGCGCTGGCGCCGCTGGTT
GCGTTCGTTGCACTGCCGCTGCCGCGTGTGGAAGGTCTGCCGGATGGTGCCGAATC
GACCAACGACGTTCCGCATGATCGTCCGGACATGGTCGAACTGCATCGTCGCGCCT
TTGATGGCCTGGCCGCACCGTTTAGCGAATTTCTGGGTACGGCCTGCGCAGATTGG
GTCATTGTGGACGTTTTTCACCACTGGGCGGCGGCGGCGGCGCTGGAACATAAAG
TGCCGTGTGCGATGATGCTGCTGGGTTCCGCCCACATGATTGCTTCAATCGCGGAT
CGTCGCCTGGAACGTGCCGAAACCGAAAGTCCGGCGGCGGCAGGCCAGGGTCGTC
CGGCGGCGGCACCGACCTTTGAAGTGGCACGTATGAAACTGATTCGCACGAAATA
A
HV1 UDP-glycosyltransferase: Amino Acid Sequence (SEQ ID NO: 7)
MDGNSSSSPLHVVICPWLALGHLLPCLDIAERLASRGHRVSFVSTPRNIARLPPLRPAV
APLVDFVALPLPHVDGLPEGAESTNDVPYDKFELHRKAFDGLAAPFSEFLRAACAEGA
GSRPDWLIVDTFHHWAAAAAVENKVPCVMLLLGAATVIAGFARGVSEHAAAAVGKE
RPAAEAPSFETERRKLMTTQNASGMTVAERYFLTLMRSDLVAIRSCAEWEPESVAALT
TLAGKPVVPLGLLPPSPEGGRGVSKEDAAVRWLDAQPAKSVVYVALGSEVPLRAEQV
HELALGLELSGARFLWALRKPTDAPDAAVLPPGFEERTRGRGLVVTGWVPQIGVLAH
GAVAAFLTHCGWNSTIEGLLFGHPLIMLPISSDQGPNARLMEGRKVGMQVPRDESDGS
FRREDVAATVRAVAVEEDGRRVFTANAKKMQEIVADGACHERCIDGFIQQLRSYKA
HV1 UDP-glycosyltransferase: DNA Sequence (SEQ ID NO: 8)
ATGGATGGTAACTCCTCCTCCTCGCCGCTGCATGTGGTCATTTGTCCGTGGCTGGC
TCTGGGTCACCTGCTGCCGTGTCTGGATATTGCTGAACGTCTGGCGTCACGCGGCC
ATCGTGTCAGTTTTGTGTCCACCCCGCGCAACATTGCCCGTCTGCCGCCGCTGCGT
CCGGCTGTTGCACCGCTGGTTGATTTCGTCGCACTGCCGCTGCCGCATGTTGACGG
TCTGCCGGAGGGTGCGGAATCGACCAATGATGTGCCGTATGACAAATTTGAACTG
CACCGTAAGGCGTTCGATGGTCTGGCGGCCCCGTTTAGCGAATTTCTGCGTGCAGC
TTGCGCAGAAGGTGCAGGTTCTCGCCCGGATTGGCTGATTGTGGACACCTTTCATC
ACTGGGCGGCGGCGGCGGCGGTGGAAAACAAAGTGCCGTGTGTTATGCTGCTGCT
GGGTGCAGCAACGGTGATCGCTGGTTTCGCGCGTGGTGTTAGCGAACATGCGGCG
GCGGCGGTGGGTAAAGAACGTCCGGCTGCGGAAGCCCCGAGTTTTGAAACCGAAC
GTCGCAAGCTGATGACCACGCAGAATGCCTCCGGCATGACCGTGGCAGAACGCTA
TTTCCTGACGCTGATGCGTAGCGATCTGGTTGCCATCCGCTCTTGCGCAGAATGGG
AACCGGAAAGCGTGGCAGCACTGACCACGCTGGCAGGTAAACCGGTGGTTCCGCT
GGGTCTGCTGCCGCCGAGTCCGGAAGGCGGTCGTGGCGTTTCCAAAGAAGATGCT
GCGGTCCGTTGGCTGGACGCACAGCCGGCAAAGTCAGTCGTGTACGTCGCACTGG
GTTCGGAAGTGCCGCTGCGTGCGGAACAAGTTCACGAACTGGCACTGGGCCTGGA
ACTGAGCGGTGCTCGCTTTCTGTGGGCGCTGCGTAAACCGACCGATGCACCGGAC
GCCGCAGTGCTGCCGCCGGGTTTCGAAGAACGTACCCGCGGCCGTGGTCTGGTTGT
CACGGGTTGGGTGCCGCAGATTGGCGTTCTGGCTCATGGTGCGGTGGCTGCGTTTC
TGACCCACTGTGGCTGGAACTCTACGATCGAAGGCCTGCTGTTCGGTCATCCGCTG
ATTATGCTGCCGATCAGCTCTGATCAGGGTCCGAATGCGCGCCTGATGGAAGGCC
GTAAAGTCGGTATGCAAGTGCCGCGTGATGAATCAGACGGCTCGTTTCGTCGCGA
AGATGTTGCCGCAACCGTCCGCGCCGTGGCAGTTGAAGAAGACGGTCGTCGCGTC
TTCACGGCTAACGCGAAAAAGATGCAAGAAATTGTGGCCGATGGCGCATGCCACG
AACGTTGTATTGACGGTTTTATCCAGCAACTGCGCAGTTACAAGGCGTAA
UGT76G1: Amino Acid Sequence (SEQ ID NO: 9)
MENKTETTVRRRRRIILFPVPFQGHINPILQLANVLYSKGFSITIFHTNFNKPKTSNYPHF
TFRFILDNDPQDERISNLPTHGPLAGMRIPIINEHGADELRRELELLMLASEEDEEVSCLI
TDALWYFAQSVADSLNLRRLVLMTSSLFNFHAHVSLPQFDELGYLDPDDKTRLEEQA
SGFPMLKVKDIKSAYSNWQILKEILGKMIKQTKASSGVIWNSFKELEESELETVIREIPA
PSFLIPLPKHLTASSSSLLDHDRTVFQWLDQQPPSSVLYVSFGSTSEVDEKDFLEIARGL
VDSKQSFLWVVRPGFVKGSTWVEPLPDGFLGERGRIVKWVPQQEVLAHGAIGAFWT
HSGWNSTLESVCEGVPMIFSDFGLDQPLNARYMSDVLKVGVYLENGWERGEIANAIR
RVMVDEEGEYIRQNARVLKQKADVSLMKGGSSYESLESLVSYISSL
UGT76G1: DNA Sequence (SEQ ID NO: 10)
ATGGAGAATAAGACAGAAACAACCGTAAGACGGAGGCGGAGGATTATCTTGTTCC
CTGTACCATTTCAGGGCCATATTAATCCGATCCTCCAATTAGCAAACGTCCTCTAC
TCCAAGGGATTTTCAATAACAATCTTCCATACTAACTTTAACAAGCCTAAAACGAG
TAATTATCCTCACTTTACATTCAGGTTCATTCTAGACAACGACCCTCAGGATGAGC
GTATCTCAAATTTACCTACGCATGGCCCCTTGGCAGGTATGCGAATACCAATAATC
AATGAGCATGGAGCCGATGAACTCCGTCGCGAGTTAGAGCTTCTCATGCTCGCAA
GTGAGGAAGACGAGGAAGTTTCGTGCCTAATAACTGATGCGCTTTGGTACTTCGCC
CAATCAGTCGCAGACTCACTGAATCTACGCCGTTTGGTCCTTATGACAAGTTCATT
ATTCAACTTTCACGCACATGTATCACTGCCGCAATTTGACGAGTTGGGTTACCTGG
ACCCGGATGACAAAACGCGATTGGAGGAACAAGCGTCGGGCTTCCCCATGCTGAA
AGTCAAAGATATTAAGAGCGCTTATAGTAATTGGCAAATTCTGAAAGAAATTCTC
GGAAAAATGATAAAGCAAACCAAAGCGTCCTCTGGAGTAATCTGGAACTCCTTCA
AGGAGTTAGAGGAATCTGAACTTGAAACGGTCATCAGAGAAATCCCCGCTCCCTC
GTTCTTAATTCCACTACCCAAGCACCTTACTGCAAGTAGCAGTTCCCTCCTAGATC
ATGACCGAACCGTGTTTCAGTGGCTGGATCAGCAACCCCCGTCGTCAGTTCTATAT
GTAAGCTTTGGGAGTACTTCGGAAGTGGATGAAAAGGACTTCTTAGAGATTGCGC
GAGGGCTCGTGGATAGCAAACAGAGCTTCCTGTGGGTAGTGAGACCGGGATTCGT
TAAGGGCTCGACGTGGGTCGAGCCGTTGCCAGATGGTTTTCTAGGGGAGAGAGGG
AGAATCGTGAAATGGGTTCCACAGCAAGAGGTTTTGGCTCACGGAGCTATAGGGG
CCTTTTGGACCCACTCTGGTTGGAATTCTACTCTTGAAAGTGTCTGTGAAGGCGTT
CCAATGATATTTTCTGATTTTGGGCTTGACCAGCCTCTAAACGCTCGCTATATGTCT
GATGTGTTGAAGGTTGGCGTGTACCTGGAGAATGGTTGGGAAAGGGGGGAAATTG
CCAACGCCATACGCCGGGTAATGGTGGACGAGGAAGGTGAGTACATACGTCAGAA
CGCTCGGGTTTTAAAACAAAAAGCGGACGTCAGCCTTATGAAGGGAGGTAGCTCC
TATGAATCCCTAGAATCCTTGGTAAGCTATATATCTTCGTTATAA
LA: Amino Acid Sequence (SEQ ID NO: 11)
MENKTETTVRRRRRIILFPVPFQGHINPILQLANVLYSKGFSITIFHTNFNKPKTSNYPHF
TFRFILDNDPQDERISNLPTHGPLAGMRIPIINEHGADELRRELELLMLASEEDEEVSCLI
TDALWYFAQSVADSLNLRRLVLMTSSLFNFHAHVSLPQFDELGYLDPDDKTRLEEQA
SGFPMLKVKDIKSAYSNWQIAKEILGKMIKQTKASSGVIWNSFKELEESELETVIREIPA
PSFLIPLPKHLTASSSSLLDHDRTVFQWLDQQPPSSVLYVSFGSTSEVDEKDFLEIARGL
VDSKQSFLWVVRPGFVKGSTWVEPLPDGFLGERGRIVKWVPQQEVLAHGAIGAFWT
HSGWNSTLESVCEGVPMIFSDFGLDQPLNARYMSDVLKVGVYLENGWERGEIANAIR
RVMVDEEGEYIRQNARVLKQKADVSLMKGGSSYESLESLVSYISSL
LA: DNA Sequence (SEQ ID NO: 12)
ATGGAGAATAAGACAGAAACAACCGTAAGACGGAGGCGGAGGATTATCTTGTTCC
CTGTACCATTTCAGGGCCATATTAATCCGATCCTCCAATTAGCAAACGTCCTCTAC
TCCAAGGGATTTTCAATAACAATCTTCCATACTAACTTTAACAAGCCTAAAACGAG
TAATTATCCTCACTTTACATTCAGGTTCATTCTAGACAACGACCCTCAGGATGAGC
GTATCTCAAATTTACCTACGCATGGCCCCTTGGCAGGTATGCGAATACCAATAATC
AATGAGCATGGAGCCGATGAACTCCGTCGCGAGTTAGAGCTTCTCATGCTCGCAA
GTGAGGAAGACGAGGAAGTTTCGTGCCTAATAACTGATGCGCTTTGGTACTTCGCC
CAATCAGTCGCAGACTCACTGAATCTACGCCGTTTGGTCCTTATGACAAGTTCATT
ATTCAACTTTCACGCACATGTATCACTGCCGCAATTTGACGAGTTGGGTTACCTGG
ACCCGGATGACAAAACGCGATTGGAGGAACAAGCGTCGGGCTTCCCCATGCTGAA
AGTCAAAGATATTAAGAGCGCTTATAGTAATTGGCAAATTGCGAAAGAAATTCTC
GGAAAAATGATAAAGCAAACCAAAGCGTCCTCTGGAGTAATCTGGAACTCCTTCA
AGGAGTTAGAGGAATCTGAACTTGAAACGGTCATCAGAGAAATCCCCGCTCCCTC
GTTCTTAATTCCACTACCCAAGCACCTTACTGCAAGTAGCAGTTCCCTCCTAGATC
ATGACCGAACCGTGTTTCAGTGGCTGGATCAGCAACCCCCGTCGTCAGTTCTATAT
GTAAGCTTTGGGAGTACTTCGGAAGTGGATGAAAAGGACTTCTTAGAGATTGCGC
GAGGGCTCGTGGATAGCAAACAGAGCTTCCTGTGGGTAGTGAGACCGGGATTCGT
TAAGGGCTCGACGTGGGTCGAGCCGTTGCCAGATGGTTTTCTAGGGGAGAGAGGG
AGAATCGTGAAATGGGTTCCACAGCAAGAGGTTTTGGCTCACGGAGCTATAGGGG
CCTTTTGGACCCACTCTGGTTGGAATTCTACTCTTGAAAGTGTCTGTGAAGGCGTT
CCAATGATATTTTCTGATTTTGGGCTTGACCAGCCTCTAAACGCTCGCTATATGTCT
GATGTGTTGAAGGTTGGCGTGTACCTGGAGAATGGTTGGGAAAGGGGGGAAATTG
CCAACGCCATACGCCGGGTAATGGTGGACGAGGAAGGTGAGTACATACGTCAGAA
CGCTCGGGTTTTAAAACAAAAAGCGGACGTCAGCCTTATGAAGGGAGGTAGCTCC
TATGAATCCCTAGAATCCTTGGTAAGCTATATATCTTCGTTATAA
CP1: Amino Acid Sequence (SEQ ID NO: 13)
MNWQILKEILGKMIKQTKASSGVIWNSFKELEESELETVIREIPAPSFLIPLPKHLTASSS
SLLDHDRTVFQWLDQQPPSSVLYVSFGSTSEVDEKDFLEIARGLVDSKQSFLWVVRPG
FVKGSTWVEPLPDGFLGERGRIVKWVPQQEVLAHGAIGAFWTHSGWNSTLESVCEGV
PMIFSDFGLDQPLNARYMSDVLKVGVYLENGWERGEIANAIRRVMVDEEGEYIRQNA
RVLKQKADVSLMKGGSSYESLESLVSYISSLENKTETTVRRRRRIILFPVPFQGHINPIL
QLANVLYSKGFSITIFHTNFNKPKTSNYPHFTFRFILDNDPQDERISNLPTHGPLAGMRI
PIINEHGADELRRELELLMLASEEDEEVSCLITDALWYFAQSVADSLNLRRLVLMTSSL
FNFHAHVSLPQFDELGYLDPDDKTRLEEQASGFPMLKVKDIKSAYS
CP1: DNA Sequence (SEQ ID NO: 14)
ATGAACTGGCAAATCCTGAAAGAAATCCTGGGTAAAATGATCAAACAAACCAAAG
CGTCGTCGGGCGTTATCTGGAACTCCTTCAAAGAACTGGAAGAATCAGAACTGGA
AACCGTTATTCGCGAAATCCCGGCTCCGTCGTTCCTGATTCCGCTGCCGAAACATC
TGACCGCGAGCAGCAGCAGCCTGCTGGATCACGACCGTACGGTCTTTCAGTGGCT
GGATCAGCAACCGCCGTCATCGGTGCTGTATGTTTCATTCGGTAGCACCTCTGAAG
TCGATGAAAAAGACTTTCTGGAAATCGCTCGCGGCCTGGTGGATAGTAAACAGTC
CTTCCTGTGGGTGGTTCGTCCGGGTTTTGTGAAAGGCAGCACGTGGGTTGAACCGC
TGCCGGATGGCTTCCTGGGTGAACGCGGCCGTATTGTCAAATGGGTGCCGCAGCA
AGAAGTGCTGGCACATGGTGCTATCGGCGCGTTTTGGACCCACTCTGGTTGGAACA
GTACGCTGGAATCCGTTTGCGAAGGTGTCCCGATGATTTTCAGCGATTTTGGCCTG
GACCAGCCGCTGAATGCCCGCTATATGTCTGATGTTCTGAAAGTCGGTGTGTACCT
GGAAAACGGTTGGGAACGTGGCGAAATTGCGAATGCCATCCGTCGCGTTATGGTC
GATGAAGAAGGCGAATACATTCGCCAGAACGCTCGTGTCCTGAAACAAAAAGCGG
ACGTGAGCCTGATGAAAGGCGGTAGCTCTTATGAATCACTGGAATCGCTGGTTAG
CTACATCAGTTCCCTGGAAAATAAAACCGAAACCACGGTGCGTCGCCGTCGCCGT
ATTATCCTGTTCCCGGTTCCGTTTCAGGGTCATATTAACCCGATCCTGCAACTGGC
GAATGTTCTGTATTCAAAAGGCTTTTCGATCACCATCTTCCATACGAACTTCAACA
AACCGAAAACCAGTAACTACCCGCACTTTACGTTCCGCTTTATTCTGGATAACGAC
CCGCAGGATGAACGTATCTCCAATCTGCCGACCCACGGCCCGCTGGCCGGTATGC
GCATTCCGATTATCAATGAACACGGTGCAGATGAACTGCGCCGTGAACTGGAACT
GCTGATGCTGGCCAGTGAAGAAGATGAAGAAGTGTCCTGTCTGATCACCGACGCA
CTGTGGTATTTCGCCCAGAGCGTTGCAGATTCTCTGAACCTGCGCCGTCTGGTCCT
GATGACGTCATCGCTGTTCAATTTTCATGCGCACGTTTCTCTGCCGCAATTTGATGA
ACTGGGCTACCTGGACCCGGATGACAAAACCCGTCTGGAAGAACAAGCCAGTGGT
TTTCCGATGCTGAAAGTCAAAGACATTAAATCCGCCTATTCGTAA
Gtf180-ΔN glucansucrase (IX14): Amino Acid Sequence (SEQ ID NO: 15)
INGQQYYIDPTTGQPRKNFLLQNGNDWIYFDKDTGAGTNALKLQFDKGTISADEQYR
RGNEAYSYDDKSIENVNGYLTADTWYRPKQILKDGTTWTDSKETDMRPILMVWWPN
TVTQAYYLNYMKQYGNLLPASLPSFSTDADSAELNHYSELVQQNIEKRISETGSTDWL
RTLMHEFVTKNSMWNKDSENVDYGGLQLQGGFLKYVNSDLTKYANSDWRLMNRTA
TNIDGKNYGGAEFLLANDIDNSNPVVQAEELNWLYYLMNFGTITGNNPEANFDGIRV
DAVDNVDVDLLSIARDYFNAAYNMEQSDASANKHINILEDWGWDDPAYVNKIGNPQ
LTMDDRLRNAIMDTLSGAPDKNQALNKLITQSLVNRANDNTENAVIPSYNFVRAHDS
NAQDQIRQAIQAATGKPYGEFNLDDEKKGMEAYINDQNSTNKKWNLYNMPSAYTILL
TNKDSVPRVYYGDLYQDGGQYMEHKTRYFDTITNLLKTRVKYVAGGQTMSVDKNGI
LTNVRFGKGAMNATDTGTDETRTEGIGVVISNNTNLKLNDGESVVLHMGAAHKNQK
YRAVILTTEDGVKNYTNDTDAPVAYTDANGDLHFTNTNLDGQQYTAVRGYANPDVT
GYLAVWVPAGAADDQDARTAPSDEAHTTKTAYRSNAALDSNVIYEGFSNFIYWPTTE
SERTNVRIAQNADLFKSWGITTFELAPQYNSSKDGTFLDSIIDNGYAFTDRYDLGMSTP
NKYGSDEDLRNALQALHKAGLQAIADWVPDQIYNLPGKEAVTVTRSDDHGTTWEVS
PIKNVVYITNTIGGGEYQKKYGGEFLDTLQKEYPQLFSQVYPVTQTTIDPSVKIKEWSA
KYFNGTNILHRGAGYVLRSNDGKYYNLGTSTQQFLPSQLSVQDNEGYGFVKEGNNY
HYYDENKQMVKDAFIQDSVGNWYYFDKNGNMVANQSPVEISSNGASGTYLFLNNGT
SFRSGLVKTDAGTYYYDGDGRMVRNQTVSDGAMTYVLDENGKLVSESFDSSATEAH
PLKPGDLNGQK
Gtf180-ΔN glucansucrase (IX14): DNA Sequence (SEQ ID NO: 16)
ATCAATGGCCAGCAGTATTATATCGATCCGACGACCGGTCAGCCTCGCAAAAACT
TTTTGCTGCAAAATGGGAACGACTGGATTTACTTTGACAAAGATACCGGGGCCGG
TACGAATGCCCTCAAACTGCAATTTGACAAGGGCACTATCAGCGCGGACGAACAG
TACCGACGTGGTAATGAAGCGTACAGCTATGATGATAAATCTATTGAAAATGTCA
ATGGGTATCTGACAGCAGATACTTGGTATCGCCCCAAGCAGATTCTGAAAGACGG
CACCACATGGACGGATAGCAAAGAAACTGACATGCGCCCGATCTTAATGGTCTGG
TGGCCCAATACCGTGACGCAGGCCTACTATCTGAATTACATGAAACAGTATGGAA
ACCTTCTGCCGGCCTCTCTGCCATCTTTTAGCACCGATGCTGATTCCGCTGAATTAA
ACCATTATAGCGAGCTAGTTCAACAGAACATCGAGAAAAGAATTAGTGAAACGGG
AAGCACTGACTGGCTGCGGACCTTGATGCATGAATTTGTGACGAAAAACAGCATG
TGGAACAAAGATTCTGAAAATGTAGATTATGGTGGCCTCCAACTTCAGGGGGGTT
TCCTGAAATACGTGAACTCCGATTTAACTAAGTACGCCAATAGCGATTGGAGACT
GATGAACCGCACCGCGACGAACATTGATGGCAAAAATTACGGAGGTGCCGAATTT
CTTTTGGCCAACGACATTGACAATAGCAACCCAGTAGTTCAAGCAGAGGAACTGA
ACTGGCTGTATTACCTCATGAACTTTGGGACCATTACCGGCAATAATCCTGAAGCT
AATTTTGATGGGATTCGAGTTGATGCGGTGGATAATGTGGACGTTGATCTTCTGTC
TATTGCTCGCGACTACTTTAATGCAGCATACAACATGGAACAATCAGATGCTTCGG
CCAACAAACATATCAACATTCTCGAAGATTGGGGCTGGGATGACCCAGCGTACGT
TAACAAGATTGGCAACCCTCAGTTAACGATGGACGACCGCCTGCGTAACGCGATT
ATGGATACGCTGTCAGGTGCGCCAGACAAAAATCAGGCGCTGAACAAGCTGATCA
CACAATCGCTGGTGAACAGAGCGAACGATAATACCGAAAACGCGGTTATTCCATC
CTACAATTTTGTCCGCGCTCACGATAGTAATGCCCAGGATCAAATTCGACAGGCAA
TCCAGGCGGCCACAGGTAAACCGTACGGAGAGTTTAATCTCGACGACGAAAAAAA
AGGTATGGAAGCGTACATTAACGACCAAAATTCGACCAACAAGAAATGGAACCTG
TACAACATGCCTTCCGCGTACACGATTCTTCTGACCAACAAAGATAGCGTACCGAG
GGTGTATTACGGGGATTTATACCAGGATGGCGGCCAGTACATGGAGCACAAGACC
CGCTATTTTGACACCATCACAAACCTGCTGAAAACCCGTGTAAAATATGTAGCTGG
CGGTCAGACCATGAGTGTTGATAAAAATGGCATTCTGACCAATGTTCGTTTTGGTA
AAGGAGCGATGAATGCTACGGATACCGGCACCGACGAGACACGTACGGAGGGCA
TTGGTGTTGTAATATCGAACAATACCAACTTGAAGCTTAACGATGGAGAAAGCGT
GGTACTGCACATGGGTGCGGCGCATAAAAATCAAAAATACCGTGCGGTGATCTTG
ACGACGGAAGATGGAGTAAAAAATTATACGAACGATACAGACGCACCCGTGGCCT
ACACCGATGCTAACGGCGATCTGCATTTCACGAATACCAATCTGGATGGCCAGCA
ATATACTGCGGTACGCGGTTATGCTAACCCGGATGTGACCGGCTACCTTGCTGTTT
GGGTGCCGGCAGGGGCGGCGGATGATCAGGATGCCCGCACTGCTCCGAGCGATGA
GGCGCATACGACAAAAACTGCGTATAGATCGAATGCAGCCTTGGATTCTAACGTT
ATATACGAAGGTTTTAGCAACTTTATTTATTGGCCGACTACCGAAAGCGAACGAAC
AAACGTACGCATTGCTCAAAATGCGGACCTGTTTAAAAGCTGGGGAATCACCACC
TTTGAACTGGCCCCGCAATACAATTCATCAAAAGACGGCACCTTTCTGGATTCAAT
TATTGATAACGGCTATGCATTTACCGATCGTTACGATCTGGGGATGTCTACCCCGA
ACAAATACGGCTCTGATGAGGATCTCCGCAATGCGCTACAAGCCTTACACAAGGC
CGGACTCCAAGCGATTGCTGACTGGGTGCCGGATCAGATTTATAATCTGCCAGGTA
AGGAAGCTGTGACCGTAACCCGTAGCGATGACCACGGCACCACGTGGGAAGTCTC
ACCGATCAAGAACGTTGTCTACATCACGAATACCATTGGTGGCGGTGAATACCAG
AAGAAATATGGTGGCGAATTTTTGGACACCTTGCAAAAAGAATACCCGCAGCTGT
TCTCACAGGTATACCCAGTGACCCAAACCACCATCGATCCGAGCGTCAAAATAAA
AGAATGGAGTGCAAAGTATTTTAACGGCACAAATATTCTTCATCGGGGCGCAGGG
TACGTACTCCGTAGCAATGACGGCAAATACTATAATCTCGGAACCTCTACTCAGCA
GTTCTTGCCGAGCCAGCTGTCAGTTCAAGATAACGAGGGATATGGGTTTGTCAAA
GAAGGTAACAATTATCATTACTATGATGAGAATAAACAAATGGTAAAGGACGCAT
TTATCCAGGACTCTGTTGGTAATTGGTATTACTTTGACAAGAACGGTAACATGGTG
GCAAACCAGTCCCCGGTGGAAATCAGTTCAAATGGGGCGTCGGGCACGTATCTCT
TCCTGAATAACGGCACCTCCTTCCGTAGTGGCTTAGTTAAGACCGATGCAGGAACC
TACTACTATGATGGGGACGGTCGTATGGTGCGGAATCAAACGGTTTCTGACGGTG
CCATGACCTACGTGTTGGACGAAAACGGAAAACTCGTGTCCGAATCTTTTGATAGT
TCGGCGACCGAAGCCCACCCACTTAAGCCAGGCGACCTCAACGGCCAGAAGTAA
UGT76G1-AtSUS1 fusion enzyme (GS): Amino Acid Sequence (SEQ ID NO: 17)
MENKTETTVRRRRRIILFPVPFQGHINPILQLANVLYSKGFSITIFHTNFNKPKTSNYPHF
TFRFILDNDPQDERISNLPTHGPLAGMRIPIINEHGADELRRELELLMLASEEDEEVSCLI
TDALWYFAQSVADSLNLRRLVLMTSSLFNFHAHVSLPQFDELGYLDPDDKTRLEEQA
SGFPMLKVKDIKSAYSNWQILKEILGKMIKQTKASSGVIWNSFKELEESELETVIREIPA
PSFLIPLPKHLTASSSSLLDHDRTVFQWLDQQPPSSVLYVSFGSTSEVDEKDFLEIARGL
VDSKQSFLWVVRPGFVKGSTWVEPLPDGFLGERGRIVKWVPQQEVLAHGAIGAFWT
HSGWNSTLESVCEGVPMIFSDFGLDQPLNARYMSDVLKVGVYLENGWERGEIANAIR
RVMVDEEGEYIRQNARVLKQKADVSLMKGGSSYESLESLVSYISSLGSGANAERMITR
VHSQRERLNETLVSERNEVLALLSRVEAKGKGILQQNQIIAEFEALPEQTRKKLEGGPF
FDLLKSTQEAIVLPPWVALAVRPRPGVWEYLRVNLHALVVEELQPAEFLHFKEELVD
GVKNGNFTLELDFEPFNASIPRPTLHKYIGNGVDFLNRHLSAKLFHDKESLLPLLKFLR
LHSHQGKNLMLSEKIQNLNTLQHTLRKAEEYLAELKSETLYEEFEAKFEEIGLERGWG
DNAERVLDMIRLLLDLLEAPDPCTLETFLGRVPMVFNVVILSPHGYFAQDNVLGYPDT
GGQVVYILDQVRALEIEMLQRIKQQGLNIKPRILILTRLLPDAVGTTCGERLERVYDSE
YCDILRVPFRTEKGIVRKWISRFEVWPYLETYTEDAAVELSKELNGKPDLIIGNYSDGN
LVASLLAHKLGVTQCTIAHALEKTKYPDSDIYWKKLDDKYHFSCQFTADIFAMNHTD
FIITSTFQEIAGSKETVGQYESHTAFTLPGLYRVVHGIDVFDPKFNIVSPGADMSIYFPYT
EEKRRLTKFHSEIEELLYSDVENKEHLCVLKDKKKPILFTMARLDRVKNLSGLVEWYG
KNTRLRELANLVVVGGDRRKESKDNEEKAEMKKMYDLIEEYKLNGQFRWISSQMDR
VRNGELYRYICDTKGAFVQPALYEAFGLTVVEAMTCGLPTFATCKGGPAEIIVHGKSG
FHIDPYHGDQAADTLADFFTKCKEDPSHWDEISKGGLQRIEEKYTWQIYSQRLLTLTG
VYGFWKHVSNLDRLEARRYLEMFYALKYRPLAQAVPLAQDD
UGT76G1-AtSUS1 fusion enzyme (GS): DNA Sequence (SEQ ID NO: 18)
ATGGAGAATAAGACAGAAACAACCGTAAGACGGAGGCGGAGGATTATCTTGTTCC
CTGTACCATTTCAGGGCCATATTAATCCGATCCTCCAATTAGCAAACGTCCTCTAC
TCCAAGGGATTTTCAATAACAATCTTCCATACTAACTTTAACAAGCCTAAAACGAG
TAATTATCCTCACTTTACATTCAGGTTCATTCTAGACAACGACCCTCAGGATGAGC
GTATCTCAAATTTACCTACGCATGGCCCCTTGGCAGGTATGCGAATACCAATAATC
AATGAGCATGGAGCCGATGAACTCCGTCGCGAGTTAGAGCTTCTCATGCTCGCAA
GTGAGGAAGACGAGGAAGTTTCGTGCCTAATAACTGATGCGCTTTGGTACTTCGCC
CAATCAGTCGCAGACTCACTGAATCTACGCCGTTTGGTCCTTATGACAAGTTCATT
ATTCAACTTTCACGCACATGTATCACTGCCGCAATTTGACGAGTTGGGTTACCTGG
ACCCGGATGACAAAACGCGATTGGAGGAACAAGCGTCGGGCTTCCCCATGCTGAA
AGTCAAAGATATTAAGAGCGCTTATAGTAATTGGCAAATTCTGAAAGAAATTCTC
GGAAAAATGATAAAGCAAACCAAAGCGTCCTCTGGAGTAATCTGGAACTCCTTCA
AGGAGTTAGAGGAATCTGAACTTGAAACGGTCATCAGAGAAATCCCCGCTCCCTC
GTTCTTAATTCCACTACCCAAGCACCTTACTGCAAGTAGCAGTTCCCTCCTAGATC
ATGACCGAACCGTGTTTCAGTGGCTGGATCAGCAACCCCCGTCGTCAGTTCTATAT
GTAAGCTTTGGGAGTACTTCGGAAGTGGATGAAAAGGACTTCTTAGAGATTGCGC
GAGGGCTCGTGGATAGCAAACAGAGCTTCCTGTGGGTAGTGAGACCGGGATTCGT
TAAGGGCTCGACGTGGGTCGAGCCGTTGCCAGATGGTTTTCTAGGGGAGAGAGGG
AGAATCGTGAAATGGGTTCCACAGCAAGAGGTTTTGGCTCACGGAGCTATAGGGG
CCTTTTGGACCCACTCTGGTTGGAATTCTACTCTTGAAAGTGTCTGTGAAGGCGTT
CCAATGATATTTTCTGATTTTGGGCTTGACCAGCCTCTAAACGCTCGCTATATGTCT
GATGTGTTGAAGGTTGGCGTGTACCTGGAGAATGGTTGGGAAAGGGGGGAAATTG
CCAACGCCATACGCCGGGTAATGGTGGACGAGGAAGGTGAGTACATACGTCAGAA
CGCTCGGGTTTTAAAACAAAAAGCGGACGTCAGCCTTATGAAGGGAGGTAGCTCC
TATGAATCCCTAGAATCCTTGGTAAGCTATATATCTTCGTTAGGTTCTGGTGCAAA
CGCTGAACGTATGATAACGCGCGTCCACAGCCAACGTGAGCGTTTGAACGAAACG
CTTGTTTCTGAGAGAAACGAAGTCCTTGCCTTGCTTTCCAGGGTTGAAGCCAAAGG
TAAAGGTATTTTACAACAAAACCAGATCATTGCTGAATTCGAAGCTTTGCCTGAAC
AAACCCGGAAGAAACTTGAAGGTGGTCCTTTCTTTGACCTTCTCAAATCCACTCAG
GAAGCAATTGTGTTGCCACCATGGGTTGCTCTAGCTGTGAGGCCAAGGCCTGGTGT
TTGGGAATACTTACGAGTCAATCTCCATGCTCTTGTCGTTGAAGAACTCCAACCTG
CTGAGTTTCTTCATTTCAAGGAAGAACTCGTTGATGGAGTTAAGAATGGTAATTTC
ACTCTTGAGCTTGATTTCGAGCCATTCAATGCGTCTATCCCTCGTCCAACACTCCAC
AAATACATTGGAAATGGTGTTGACTTCCTTAACCGTCATTTATCGGCTAAGCTCTT
CCATGACAAGGAGAGTTTGCTTCCATTGCTTAAGTTCCTTCGTCTTCACAGCCACC
AGGGCAAGAACCTGATGTTGAGCGAGAAGATTCAGAACCTCAACACTCTGCAACA
CACCTTGAGGAAAGCAGAAGAGTATCTAGCAGAGCTTAAGTCCGAAACACTGTAT
GAAGAGTTTGAGGCCAAGTTTGAGGAGATTGGTCTTGAGAGGGGATGGGGAGACA
ATGCAGAGCGTGTCCTTGACATGATACGTCTTCTTTTGGACCTTCTTGAGGCGCCT
GATCCTTGCACTCTTGAGACTTTTCTTGGAAGAGTACCAATGGTGTTCAACGTTGT
GATCCTCTCTCCACATGGTTACTTTGCTCAGGACAATGTTCTTGGTTACCCTGACAC
TGGTGGACAGGTTGTTTACATTCTTGATCAAGTTCGTGCTCTGGAGATAGAGATGC
TTCAACGTATTAAGCAACAAGGACTCAACATTAAACCAAGGATTCTCATTCTAACT
CGACTTCTACCTGATGCGGTAGGAACTACATGCGGTGAACGTCTCGAGAGAGTTT
ATGATTCTGAGTACTGTGATATTCTTCGTGTGCCCTTCAGAACAGAGAAGGGTATT
GTTCGCAAATGGATCTCAAGGTTCGAAGTCTGGCCATATCTAGAGACTTACACCGA
GGATGCTGCGGTTGAGCTATCGAAAGAATTGAATGGCAAGCCTGACCTTATCATT
GGTAACTACAGTGATGGAAATCTTGTTGCTTCTTTATTGGCTCACAAACTTGGTGT
CACTCAGTGTACCATTGCTCATGCTCTTGAGAAAACAAAGTACCCGGATTCTGATA
TCTACTGGAAGAAGCTTGACGACAAGTACCATTTCTCATGCCAGTTCACTGCGGAT
ATTTTCGCAATGAACCACACTGATTTCATCATCACTAGTACTTTCCAAGAAATTGC
TGGAAGCAAAGAAACTGTTGGGCAGTATGAAAGCCACACAGCCTTTACTCTTCCC
GGATTGTATCGAGTTGTTCACGGGATTGATGTGTTTGATCCCAAGTTCAACATTGT
CTCTCCTGGTGCTGATATGAGCATCTACTTCCCTTACACAGAGGAGAAGCGTAGAT
TGACTAAGTTCCACTCTGAGATCGAGGAGCTCCTCTACAGCGATGTTGAGAACAA
AGAGCACTTATGTGTGCTCAAGGACAAGAAGAAGCCGATTCTCTTCACAATGGCT
AGGCTTGATCGTGTCAAGAACTTGTCAGGTCTTGTTGAGTGGTACGGGAAGAACA
CCCGCTTGCGTGAGCTAGCTAACTTGGTTGTTGTTGGAGGAGACAGGAGGAAAGA
GTCAAAGGACAATGAAGAGAAAGCAGAGATGAAGAAAATGTATGATCTCATTGA
GGAATACAAGCTAAACGGTCAGTTCAGGTGGATCTCCTCTCAGATGGACCGGGTA
AGGAACGGTGAGCTGTACCGGTACATCTGTGACACCAAGGGTGCTTTTGTCCAAC
CTGCATTATATGAAGCCTTTGGGTTAACTGTTGTGGAGGCTATGACTTGTGGTTTA
CCGACTTTCGCCACTTGCAAAGGTGGTCCAGCTGAGATCATTGTGCACGGTAAATC
GGGTTTCCACATTGACCCTTACCATGGTGATCAGGCTGCTGATACTCTTGCTGATTT
CTTCACCAAGTGTAAGGAGGATCCATCTCACTGGGATGAGATCTCAAAAGGAGGG
CTTCAGAGGATTGAGGAGAAATACACTTGGCAAATCTATTCACAGAGGCTCTTGA
CATTGACTGGTGTGTATGGATTCTGGAAGCATGTCTCGAACCTTGACCGTCTTGAG
GCTCGCCGTTACCTTGAAATGTTCTATGCATTGAAGTATCGCCCATTGGCTCAGGC
TGTTCCTCTTGCACAAGATGATTGA
LA-AtSUS1 fusion enzyme: Amino Acid Sequence (SEQ ID NO: 19)
MENKTETTVRRRRRIILFPVPFQGHINPILQLANVLYSKGFSITIFHTNFNKPKTSNYPHF
TFRFILDNDPQDERISNLPTHGPLAGMRIPIINEHGADELRRELELLMLASEEDEEVSCLI
TDALWYFAQSVADSLNLRRLVLMTSSLFNFHAHVSLPQFDELGYLDPDDKTRLEEQA
SGFPMLKVKDIKSAYSNWQIAKEILGKMIKQTKASSGVIWNSFKELEESELETVIREIPA
PSFLIPLPKHLTASSSSLLDHDRTVFQWLDQQPPSSVLYVSFGSTSEVDEKDFLEIARGL
VDSKQSFLWVVRPGFVKGSTWVEPLPDGFLGERGRIVKWVPQQEVLAHGAIGAFWT
HSGWNSTLESVCEGVPMIFSDFGLDQPLNARYMSDVLKVGVYLENGWERGEIANAIR
RVMVDEEGEYIRQNARVLKQKADVSLMKGGSSYESLESLVSYISSLGSGANAERMITR
VHSQRERLNETLVSERNEVLALLSRVEAKGKGILQQNQIIAEFEALPEQTRKKLEGGPF
FDLLKSTQEAIVLPPWVALAVRPRPGVWEYLRVNLHALVVEELQPAEFLHFKEELVD
GVKNGNFTLELDFEPFNASIPRPTLHKYIGNGVDFLNRHLSAKLFHDKESLLPLLKFLR
LHSHQGKNLMLSEKIQNLNTLQHTLRKAEEYLAELKSETLYEEFEAKFEEIGLERGWG
DNAERVLDMIRLLLDLLEAPDPCTLETFLGRVPMVFNVVILSPHGYFAQDNVLGYPDT
GGQVVYILDQVRALEIEMLQRIKQQGLNIKPRILILTRLLPDAVGTTCGERLERVYDSE
YCDILRVPFRTEKGIVRKWISRFEVWPYLETYTEDAAVELSKELNGKPDLIIGNYSDGN
LVASLLAHKLGVTQCTIAHALEKTKYPDSDIYWKKLDDKYHFSCQFTADIFAMNHTD
FIITSTFQEIAGSKETVGQYESHTAFTLPGLYRVVHGIDVFDPKFNIVSPGADMSIYFPYT
EEKRRLTKFHSEIEELLYSDVENKEHLCVLKDKKKPILFTMARLDRVKNLSGLVEWYG
KNTRLRELANLVVVGGDRRKESKDNEEKAEMKKMYDLIEEYKLNGQFRWISSQMDR
VRNGELYRYICDTKGAFVQPALYEAFGLTVVEAMTCGLPTFATCKGGPAEIIVHGKSG
FHIDPYHGDQAADTLADFFTKCKEDPSHWDEISKGGLQRIEEKYTWQIYSQRLLTLTG
VYGFWKHVSNLDRLEARRYLEMFYALKYRPLAQAVPLAQDD
LA-AtSUS1 fusion enzyme: DNA Sequence (SEQ ID NO: 20)
ATGGAGAATAAGACAGAAACAACCGTAAGACGGAGGCGGAGGATTATCTTGTTCC
CTGTACCATTTCAGGGCCATATTAATCCGATCCTCCAATTAGCAAACGTCCTCTAC
TCCAAGGGATTTTCAATAACAATCTTCCATACTAACTTTAACAAGCCTAAAACGAG
TAATTATCCTCACTTTACATTCAGGTTCATTCTAGACAACGACCCTCAGGATGAGC
GTATCTCAAATTTACCTACGCATGGCCCCTTGGCAGGTATGCGAATACCAATAATC
AATGAGCATGGAGCCGATGAACTCCGTCGCGAGTTAGAGCTTCTCATGCTCGCAA
GTGAGGAAGACGAGGAAGTTTCGTGCCTAATAACTGATGCGCTTTGGTACTTCGCC
CAATCAGTCGCAGACTCACTGAATCTACGCCGTTTGGTCCTTATGACAAGTTCATT
ATTCAACTTTCACGCACATGTATCACTGCCGCAATTTGACGAGTTGGGTTACCTGG
ACCCGGATGACAAAACGCGATTGGAGGAACAAGCGTCGGGCTTCCCCATGCTGAA
AGTCAAAGATATTAAGAGCGCTTATAGTAATTGGCAAATTGCGAAAGAAATTCTC
GGAAAAATGATAAAGCAAACCAAAGCGTCCTCTGGAGTAATCTGGAACTCCTTCA
AGGAGTTAGAGGAATCTGAACTTGAAACGGTCATCAGAGAAATCCCCGCTCCCTC
GTTCTTAATTCCACTACCCAAGCACCTTACTGCAAGTAGCAGTTCCCTCCTAGATC
ATGACCGAACCGTGTTTCAGTGGCTGGATCAGCAACCCCCGTCGTCAGTTCTATAT
GTAAGCTTTGGGAGTACTTCGGAAGTGGATGAAAAGGACTTCTTAGAGATTGCGC
GAGGGCTCGTGGATAGCAAACAGAGCTTCCTGTGGGTAGTGAGACCGGGATTCGT
TAAGGGCTCGACGTGGGTCGAGCCGTTGCCAGATGGTTTTCTAGGGGAGAGAGGG
AGAATCGTGAAATGGGTTCCACAGCAAGAGGTTTTGGCTCACGGAGCTATAGGGG
CCTTTTGGACCCACTCTGGTTGGAATTCTACTCTTGAAAGTGTCTGTGAAGGCGTT
CCAATGATATTTTCTGATTTTGGGCTTGACCAGCCTCTAAACGCTCGCTATATGTCT
GATGTGTTGAAGGTTGGCGTGTACCTGGAGAATGGTTGGGAAAGGGGGGAAATTG
CCAACGCCATACGCCGGGTAATGGTGGACGAGGAAGGTGAGTACATACGTCAGAA
CGCTCGGGTTTTAAAACAAAAAGCGGACGTCAGCCTTATGAAGGGAGGTAGCTCC
TATGAATCCCTAGAATCCTTGGTAAGCTATATATCTTCGTTAGGTTCTGGTGCAAA
CGCTGAACGTATGATAACGCGCGTCCACAGCCAACGTGAGCGTTTGAACGAAACG
CTTGTTTCTGAGAGAAACGAAGTCCTTGCCTTGCTTTCCAGGGTTGAAGCCAAAGG
TAAAGGTATTTTACAACAAAACCAGATCATTGCTGAATTCGAAGCTTTGCCTGAAC
AAACCCGGAAGAAACTTGAAGGTGGTCCTTTCTTTGACCTTCTCAAATCCACTCAG
GAAGCAATTGTGTTGCCACCATGGGTTGCTCTAGCTGTGAGGCCAAGGCCTGGTGT
TTGGGAATACTTACGAGTCAATCTCCATGCTCTTGTCGTTGAAGAACTCCAACCTG
CTGAGTTTCTTCATTTCAAGGAAGAACTCGTTGATGGAGTTAAGAATGGTAATTTC
ACTCTTGAGCTTGATTTCGAGCCATTCAATGCGTCTATCCCTCGTCCAACACTCCAC
AAATACATTGGAAATGGTGTTGACTTCCTTAACCGTCATTTATCGGCTAAGCTCTT
CCATGACAAGGAGAGTTTGCTTCCATTGCTTAAGTTCCTTCGTCTTCACAGCCACC
AGGGCAAGAACCTGATGTTGAGCGAGAAGATTCAGAACCTCAACACTCTGCAACA
CACCTTGAGGAAAGCAGAAGAGTATCTAGCAGAGCTTAAGTCCGAAACACTGTAT
GAAGAGTTTGAGGCCAAGTTTGAGGAGATTGGTCTTGAGAGGGGATGGGGAGACA
ATGCAGAGCGTGTCCTTGACATGATACGTCTTCTTTTGGACCTTCTTGAGGCGCCT
GATCCTTGCACTCTTGAGACTTTTCTTGGAAGAGTACCAATGGTGTTCAACGTTGT
GATCCTCTCTCCACATGGTTACTTTGCTCAGGACAATGTTCTTGGTTACCCTGACAC
TGGTGGACAGGTTGTTTACATTCTTGATCAAGTTCGTGCTCTGGAGATAGAGATGC
TTCAACGTATTAAGCAACAAGGACTCAACATTAAACCAAGGATTCTCATTCTAACT
CGACTTCTACCTGATGCGGTAGGAACTACATGCGGTGAACGTCTCGAGAGAGTTT
ATGATTCTGAGTACTGTGATATTCTTCGTGTGCCCTTCAGAACAGAGAAGGGTATT
GTTCGCAAATGGATCTCAAGGTTCGAAGTCTGGCCATATCTAGAGACTTACACCGA
GGATGCTGCGGTTGAGCTATCGAAAGAATTGAATGGCAAGCCTGACCTTATCATT
GGTAACTACAGTGATGGAAATCTTGTTGCTTCTTTATTGGCTCACAAACTTGGTGT
CACTCAGTGTACCATTGCTCATGCTCTTGAGAAAACAAAGTACCCGGATTCTGATA
TCTACTGGAAGAAGCTTGACGACAAGTACCATTTCTCATGCCAGTTCACTGCGGAT
ATTTTCGCAATGAACCACACTGATTTCATCATCACTAGTACTTTCCAAGAAATTGC
TGGAAGCAAAGAAACTGTTGGGCAGTATGAAAGCCACACAGCCTTTACTCTTCCC
GGATTGTATCGAGTTGTTCACGGGATTGATGTGTTTGATCCCAAGTTCAACATTGT
CTCTCCTGGTGCTGATATGAGCATCTACTTCCCTTACACAGAGGAGAAGCGTAGAT
TGACTAAGTTCCACTCTGAGATCGAGGAGCTCCTCTACAGCGATGTTGAGAACAA
AGAGCACTTATGTGTGCTCAAGGACAAGAAGAAGCCGATTCTCTTCACAATGGCT
AGGCTTGATCGTGTCAAGAACTTGTCAGGTCTTGTTGAGTGGTACGGGAAGAACA
CCCGCTTGCGTGAGCTAGCTAACTTGGTTGTTGTTGGAGGAGACAGGAGGAAAGA
GTCAAAGGACAATGAAGAGAAAGCAGAGATGAAGAAAATGTATGATCTCATTGA
GGAATACAAGCTAAACGGTCAGTTCAGGTGGATCTCCTCTCAGATGGACCGGGTA
AGGAACGGTGAGCTGTACCGGTACATCTGTGACACCAAGGGTGCTTTTGTCCAAC
CTGCATTATATGAAGCCTTTGGGTTAACTGTTGTGGAGGCTATGACTTGTGGTTTA
CCGACTTTCGCCACTTGCAAAGGTGGTCCAGCTGAGATCATTGTGCACGGTAAATC
GGGTTTCCACATTGACCCTTACCATGGTGATCAGGCTGCTGATACTCTTGCTGATTT
CTTCACCAAGTGTAAGGAGGATCCATCTCACTGGGATGAGATCTCAAAAGGAGGG
CTTCAGAGGATTGAGGAGAAATACACTTGGCAAATCTATTCACAGAGGCTCTTGA
CATTGACTGGTGTGTATGGATTCTGGAAGCATGTCTCGAACCTTGACCGTCTTGAG
GCTCGCCGTTACCTTGAAATGTTCTATGCATTGAAGTATCGCCCATTGGCTCAGGC
TGTTCCTCTTGCACAAGATGATTGA
CP1-AtSUS1 fusion enzyme: Amino Acid Sequence (SEQ ID NO: 21)
MNWQILKEILGKMIKQTKASSGVIWNSFKELEESELETVIREIPAPSFLIPLPKHLTASSS
SLLDHDRTVFQWLDQQPPSSVLYVSFGSTSEVDEKDFLEIARGLVDSKQSFLWVVRPG
FVKGSTWVEPLPDGFLGERGRIVKWVPQQEVLAHGAIGAFWTHSGWNSTLESVCEGV
PMIFSDFGLDQPLNARYMSDVLKVGVYLENGWERGEIANAIRRVMVDEEGEYIRQNA
RVLKQKADVSLMKGGSSYESLESLVSYISSLENKTETTVRRRRRIILFPVPFQGHINPIL
QLANVLYSKGFSITIFHTNFNKPKTSNYPHFTFRFILDNDPQDERISNLPTHGPLAGMRI
PIINEHGADELRRELELLMLASEEDEEVSCLITDALWYFAQSVADSLNLRRLVLMTSSL
FNFHAHVSLPQFDELGYLDPDDKTRLEEQASGFPMLKVKDIKSAYSGSGANAERMITR
VHSQRERLNETLVSERNEVLALLSRVEAKGKGILQQNQIIAEFEALPEQTRKKLEGGPF
FDLLKSTQEAIVLPPWVALAVRPRPGVWEYLRVNLHALVVEELQPAEFLHFKEELVD
GVKNGNFTLELDFEPFNASIPRPTLHKYIGNGVDFLNRHLSAKLFHDKESLLPLLKFLR
LHSHQGKNLMLSEKIQNLNTLQHTLRKAEEYLAELKSETLYEEFEAKFEEIGLERGWG
DNAERVLDMIRLLLDLLEAPDPCTLETFLGRVPMVFNVVILSPHGYFAQDNVLGYPDT
GGQVVYILDQVRALEIEMLQRIKQQGLNIKPRILILTRLLPDAVGTTCGERLERVYDSE
YCDILRVPFRTEKGIVRKWISRFEVWPYLETYTEDAAVELSKELNGKPDLIIGNYSDGN
LVASLLAHKLGVTQCTIAHALEKTKYPDSDIYWKKLDDKYHFSCQFTADIFAMNHTD
FIITSTFQEIAGSKETVGQYESHTAFTLPGLYRVVHGIDVFDPKFNIVSPGADMSIYFPYT
EEKRRLTKFHSEIEELLYSDVENKEHLCVLKDKKKPILFTMARLDRVKNLSGLVEWYG
KNTRLRELANLVVVGGDRRKESKDNEEKAEMKKMYDLIEEYKLNGQFRWISSQMDR
VRNGELYRYICDTKGAFVQPALYEAFGLTVVEAMTCGLPTFATCKGGPAEIIVHGKSG
FHIDPYHGDQAADTLADFFTKCKEDPSHWDEISKGGLQRIEEKYTWQIYSQRLLTLTG
VYGFWKHVSNLDRLEARRYLEMFYALKYRPLAQAVPLAQDD*
CP1-AtSUS1 fusion enzyme: DNA Sequence (SEQ ID NO: 22)
ATGAACTGGCAAATCCTGAAAGAAATCCTGGGTAAAATGATCAAACAAACCAAAG
CGTCGTCGGGCGTTATCTGGAACTCCTTCAAAGAACTGGAAGAATCAGAACTGGA
AACCGTTATTCGCGAAATCCCGGCTCCGTCGTTCCTGATTCCGCTGCCGAAACATC
TGACCGCGAGCAGCAGCAGCCTGCTGGATCACGACCGTACGGTCTTTCAGTGGCT
GGATCAGCAACCGCCGTCATCGGTGCTGTATGTTTCATTCGGTAGCACCTCTGAAG
TCGATGAAAAAGACTTTCTGGAAATCGCTCGCGGCCTGGTGGATAGTAAACAGTC
CTTCCTGTGGGTGGTTCGTCCGGGTTTTGTGAAAGGCAGCACGTGGGTTGAACCGC
TGCCGGATGGCTTCCTGGGTGAACGCGGCCGTATTGTCAAATGGGTGCCGCAGCA
AGAAGTGCTGGCACATGGTGCTATCGGCGCGTTTTGGACCCACTCTGGTTGGAACA
GTACGCTGGAATCCGTTTGCGAAGGTGTCCCGATGATTTTCAGCGATTTTGGCCTG
GACCAGCCGCTGAATGCCCGCTATATGTCTGATGTTCTGAAAGTCGGTGTGTACCT
GGAAAACGGTTGGGAACGTGGCGAAATTGCGAATGCCATCCGTCGCGTTATGGTC
GATGAAGAAGGCGAATACATTCGCCAGAACGCTCGTGTCCTGAAACAAAAAGCGG
ACGTGAGCCTGATGAAAGGCGGTAGCTCTTATGAATCACTGGAATCGCTGGTTAG
CTACATCAGTTCCCTGGAAAATAAAACCGAAACCACGGTGCGTCGCCGTCGCCGT
ATTATCCTGTTCCCGGTTCCGTTTCAGGGTCATATTAACCCGATCCTGCAACTGGC
GAATGTTCTGTATTCAAAAGGCTTTTCGATCACCATCTTCCATACGAACTTCAACA
AACCGAAAACCAGTAACTACCCGCACTTTACGTTCCGCTTTATTCTGGATAACGAC
CCGCAGGATGAACGTATCTCCAATCTGCCGACCCACGGCCCGCTGGCCGGTATGC
GCATTCCGATTATCAATGAACACGGTGCAGATGAACTGCGCCGTGAACTGGAACT
GCTGATGCTGGCCAGTGAAGAAGATGAAGAAGTGTCCTGTCTGATCACCGACGCA
CTGTGGTATTTCGCCCAGAGCGTTGCAGATTCTCTGAACCTGCGCCGTCTGGTCCT
GATGACGTCATCGCTGTTCAATTTTCATGCGCACGTTTCTCTGCCGCAATTTGATGA
ACTGGGCTACCTGGACCCGGATGACAAAACCCGTCTGGAAGAACAAGCCAGTGGT
TTTCCGATGCTGAAAGTCAAAGACATTAAATCCGCCTATTCGGGTTCTGGTGCAAA
CGCTGAACGTATGATAACGCGCGTCCACAGCCAACGTGAGCGTTTGAACGAAACG
CTTGTTTCTGAGAGAAACGAAGTCCTTGCCTTGCTTTCCAGGGTTGAAGCCAAAGG
TAAAGGTATTTTACAACAAAACCAGATCATTGCTGAATTCGAAGCTTTGCCTGAAC
AAACCCGGAAGAAACTTGAAGGTGGTCCTTTCTTTGACCTTCTCAAATCCACTCAG
GAAGCAATTGTGTTGCCACCATGGGTTGCTCTAGCTGTGAGGCCAAGGCCTGGTGT
TTGGGAATACTTACGAGTCAATCTCCATGCTCTTGTCGTTGAAGAACTCCAACCTG
CTGAGTTTCTTCATTTCAAGGAAGAACTCGTTGATGGAGTTAAGAATGGTAATTTC
ACTCTTGAGCTTGATTTCGAGCCATTCAATGCGTCTATCCCTCGTCCAACACTCCAC
AAATACATTGGAAATGGTGTTGACTTCCTTAACCGTCATTTATCGGCTAAGCTCTT
CCATGACAAGGAGAGTTTGCTTCCATTGCTTAAGTTCCTTCGTCTTCACAGCCACC
AGGGCAAGAACCTGATGTTGAGCGAGAAGATTCAGAACCTCAACACTCTGCAACA
CACCTTGAGGAAAGCAGAAGAGTATCTAGCAGAGCTTAAGTCCGAAACACTGTAT
GAAGAGTTTGAGGCCAAGTTTGAGGAGATTGGTCTTGAGAGGGGATGGGGAGACA
ATGCAGAGCGTGTCCTTGACATGATACGTCTTCTTTTGGACCTTCTTGAGGCGCCT
GATCCTTGCACTCTTGAGACTTTTCTTGGAAGAGTACCAATGGTGTTCAACGTTGT
GATCCTCTCTCCACATGGTTACTTTGCTCAGGACAATGTTCTTGGTTACCCTGACAC
TGGTGGACAGGTTGTTTACATTCTTGATCAAGTTCGTGCTCTGGAGATAGAGATGC
TTCAACGTATTAAGCAACAAGGACTCAACATTAAACCAAGGATTCTCATTCTAACT
CGACTTCTACCTGATGCGGTAGGAACTACATGCGGTGAACGTCTCGAGAGAGTTT
ATGATTCTGAGTACTGTGATATTCTTCGTGTGCCCTTCAGAACAGAGAAGGGTATT
GTTCGCAAATGGATCTCAAGGTTCGAAGTCTGGCCATATCTAGAGACTTACACCGA
GGATGCTGCGGTTGAGCTATCGAAAGAATTGAATGGCAAGCCTGACCTTATCATT
GGTAACTACAGTGATGGAAATCTTGTTGCTTCTTTATTGGCTCACAAACTTGGTGT
CACTCAGTGTACCATTGCTCATGCTCTTGAGAAAACAAAGTACCCGGATTCTGATA
TCTACTGGAAGAAGCTTGACGACAAGTACCATTTCTCATGCCAGTTCACTGCGGAT
ATTTTCGCAATGAACCACACTGATTTCATCATCACTAGTACTTTCCAAGAAATTGC
TGGAAGCAAAGAAACTGTTGGGCAGTATGAAAGCCACACAGCCTTTACTCTTCCC
GGATTGTATCGAGTTGTTCACGGGATTGATGTGTTTGATCCCAAGTTCAACATTGT
CTCTCCTGGTGCTGATATGAGCATCTACTTCCCTTACACAGAGGAGAAGCGTAGAT
TGACTAAGTTCCACTCTGAGATCGAGGAGCTCCTCTACAGCGATGTTGAGAACAA
AGAGCACTTATGTGTGCTCAAGGACAAGAAGAAGCCGATTCTCTTCACAATGGCT
AGGCTTGATCGTGTCAAGAACTTGTCAGGTCTTGTTGAGTGGTACGGGAAGAACA
CCCGCTTGCGTGAGCTAGCTAACTTGGTTGTTGTTGGAGGAGACAGGAGGAAAGA
GTCAAAGGACAATGAAGAGAAAGCAGAGATGAAGAAAATGTATGATCTCATTGA
GGAATACAAGCTAAACGGTCAGTTCAGGTGGATCTCCTCTCAGATGGACCGGGTA
AGGAACGGTGAGCTGTACCGGTACATCTGTGACACCAAGGGTGCTTTTGTCCAAC
CTGCATTATATGAAGCCTTTGGGTTAACTGTTGTGGAGGCTATGACTTGTGGTTTA
CCGACTTTCGCCACTTGCAAAGGTGGTCCAGCTGAGATCATTGTGCACGGTAAATC
GGGTTTCCACATTGACCCTTACCATGGTGATCAGGCTGCTGATACTCTTGCTGATTT
CTTCACCAAGTGTAAGGAGGATCCATCTCACTGGGATGAGATCTCAAAAGGAGGG
CTTCAGAGGATTGAGGAGAAATACACTTGGCAAATCTATTCACAGAGGCTCTTGA
CATTGACTGGTGTGTATGGATTCTGGAAGCATGTCTCGAACCTTGACCGTCTTGAG
GCTCGCCGTTACCTTGAAATGTTCTATGCATTGAAGTATCGCCCATTGGCTCAGGC
TGTTCCTCTTGCACAAGATGATTGA
EU11-AtSUS1 fusion enzyme (EUS): Amino Acid Sequence (SEQ ID NO: 23)
MDSGYSSSYAAAAGMHVVICPWLAFGHLLPCLDLAQRLASRGHRVSFVSTPRNISRLP
PVRPALAPLVAFVALPLPRVEGLPDGAESTNDVPHDRPDMVELHRRAFDGLAAPFSEF
LGTACADWVIVDVFHHWAAAAALEHKVPCAMMLLGSAHMIASIADRRLERAETESP
AAAGQGRPAAAPTFEVARMKLIRTKGSSGMSLAERFSLTLSRSSLVVGRSCVEFEPET
VPLLSTLRGKPITFLGLMPPLHEGRREDGEDATVRWLDAQPAKSVVYVALGSEVPLG
VEKVHELALGLELAGTRFLWALRKPTGVSDADLLPAGFEERTRGRGVVATRWVPQM
SILAHAAVGAFLTHCGWNSTIEGLMFGHPLIMLPIFGDQGPNARLIEAKNAGLQVARN
DGDGSFDREGVAAAIRAVAVEEESSKVFQAKAKKLQEIVADMACHERYIDGFIQQLRS
YKDGSGANAERMITRVHSQRERLNETLVSERNEVLALLSRVEAKGKGILQQNQIIAEF
EALPEQTRKKLEGGPFFDLLKSTQEAIVLPPWVALAVRPRPGVWEYLRVNLHALVVE
ELQPAEFLHFKEELVDGVKNGNFTLELDFEPFNASIPRPTLHKYIGNGVDFLNRHLSAK
LFHDKESLLPLLKFLRLHSHQGKNLMLSEKIQNLNTLQHTLRKAEEYLAELKSETLYE
EFEAKFEEIGLERGWGDNAERVLDMIRLLLDLLEAPDPCTLETFLGRVPMVENVVILSP
HGYFAQDNVLGYPDTGGQVVYILDQVRALEIEMLQRIKQQGLNIKPRILILTRLLPDAV
GTTCGERLERVYDSEYCDILRVPFRTEKGIVRKWISRFEVWPYLETYTEDAAVELSKEL
NGKPDLIIGNYSDGNLVASLLAHKLGVTQCTIAHALEKTKYPDSDIYWKKLDDKYHFS
CQFTADIFAMNHTDFIITSTFQEIAGSKETVGQYESHTAFTLPGLYRVVHGIDVFDPKFN
IVSPGADMSIYFPYTEEKRRLTKFHSEIEELLYSDVENKEHLCVLKDKKKPILFTMARL
DRVKNLSGLVEWYGKNTRLRELANLVVVGGDRRKESKDNEEKAEMKKMYDLIEEY
KLNGQFRWISSQMDRVRNGELYRYICDTKGAFVQPALYEAFGLTVVEAMTCGLPTFA
TCKGGPAEIIVHGKSGFHIDPYHGDQAADTLADFFTKCKEDPSHWDEISKGGLQRIEEK
YTWQIYSQRLLTLTGVYGFWKHVSNLDRLEARRYLEMFYALKYRPLAQAVPLAQDD
EU11-AtSUS1 fusion enzyme (EUS): DNA Sequence (SEQ ID NO: 24)
ATGGATTCGGGTTACTCTTCCTCCTATGCGGCGGCTGCGGGTATGCACGTTGTTAT
CTGTCCGTGGCTGGCTTTTGGTCACCTGCTGCCGTGCCTGGATCTGGCACAGCGTC
TGGCTTCACGCGGCCATCGTGTCAGCTTCGTGTCTACCCCGCGCAATATTTCGCGT
CTGCCGCCGGTTCGTCCGGCACTGGCTCCGCTGGTTGCATTTGTCGCTCTGCCGCT
GCCGCGCGTGGAAGGTCTGCCGGATGGTGCGGAAAGTACCAACGACGTGCCGCAT
GATCGCCCGGACATGGTTGAACTGCACCGTCGTGCATTCGATGGTCTGGCAGCACC
GTTTTCCGAATTTCTGGGTACGGCGTGCGCCGATTGGGTGATCGTTGACGTCTTTC
ATCACTGGGCGGCGGCGGCGGCGCTGGAACATAAAGTTCCGTGTGCAATGATGCT
GCTGGGCTCAGCTCACATGATTGCGTCGATCGCAGACCGTCGCCTGGAACGTGCA
GAAACCGAAAGTCCGGCTGCGGCCGGCCAGGGTCGCCCGGCAGCTGCGCCGACCT
TCGAAGTGGCCCGCATGAAACTGATTCGTACGAAAGGCAGCTCTGGTATGAGCCT
GGCAGAACGCTTTAGTCTGACCCTGTCCCGTAGTTCCCTGGTGGTTGGTCGCAGTT
GCGTTGAATTTGAACCGGAAACCGTCCCGCTGCTGTCCACGCTGCGTGGTAAACCG
ATCACCTTTCTGGGTCTGATGCCGCCGCTGCATGAAGGCCGTCGCGAAGATGGTGA
AGACGCAACGGTGCGTTGGCTGGATGCACAGCCGGCTAAAAGCGTCGTGTATGTC
GCCCTGGGCTCTGAAGTGCCGCTGGGTGTGGAAAAAGTTCACGAACTGGCACTGG
GCCTGGAACTGGCTGGCACCCGCTTCCTGTGGGCACTGCGTAAACCGACGGGTGT
GAGCGATGCGGACCTGCTGCCGGCCGGTTTTGAAGAACGTACCCGCGGCCGTGGT
GTTGTCGCAACGCGTTGGGTCCCGCAAATGAGCATTCTGGCGCATGCCGCAGTGG
GCGCCTTTCTGACCCACTGTGGTTGGAACAGCACGATCGAAGGCCTGATGTTTGGT
CACCCGCTGATTATGCTGCCGATCTTCGGCGATCAGGGTCCGAACGCACGTCTGAT
TGAAGCGAAAAATGCCGGCCTGCAAGTTGCGCGCAACGATGGCGACGGTTCTTTC
GACCGTGAGGGTGTGGCTGCGGCCATTCGCGCAGTGGCTGTTGAAGAAGAATCAT
CGAAAGTTTTTCAGGCGAAAGCCAAAAAACTGCAAGAAATCGTCGCGGATATGGC
CTGCCACGAACGCTACATTGATGGTTTCATTCAGCAACTGCGCTCCTACAAAGACG
GTTCTGGTGCAAACGCTGAACGTATGATAACGCGCGTCCACAGCCAACGTGAGCG
TTTGAACGAAACGCTTGTTTCTGAGAGAAACGAAGTCCTTGCCTTGCTTTCCAGGG
TTGAAGCCAAAGGTAAAGGTATTTTACAACAAAACCAGATCATTGCTGAATTCGA
AGCTTTGCCTGAACAAACCCGGAAGAAACTTGAAGGTGGTCCTTTCTTTGACCTTC
TCAAATCCACTCAGGAAGCAATTGTGTTGCCACCATGGGTTGCTCTAGCTGTGAGG
CCAAGGCCTGGTGTTTGGGAATACTTACGAGTCAATCTCCATGCTCTTGTCGTTGA
AGAACTCCAACCTGCTGAGTTTCTTCATTTCAAGGAAGAACTCGTTGATGGAGTTA
AGAATGGTAATTTCACTCTTGAGCTTGATTTCGAGCCATTCAATGCGTCTATCCCTC
GTCCAACACTCCACAAATACATTGGAAATGGTGTTGACTTCCTTAACCGTCATTTA
TCGGCTAAGCTCTTCCATGACAAGGAGAGTTTGCTTCCATTGCTTAAGTTCCTTCGT
CTTCACAGCCACCAGGGCAAGAACCTGATGTTGAGCGAGAAGATTCAGAACCTCA
ACACTCTGCAACACACCTTGAGGAAAGCAGAAGAGTATCTAGCAGAGCTTAAGTC
CGAAACACTGTATGAAGAGTTTGAGGCCAAGTTTGAGGAGATTGGTCTTGAGAGG
GGATGGGGAGACAATGCAGAGCGTGTCCTTGACATGATACGTCTTCTTTTGGACCT
TCTTGAGGCGCCTGATCCTTGCACTCTTGAGACTTTTCTTGGAAGAGTACCAATGG
TGTTCAACGTTGTGATCCTCTCTCCACATGGTTACTTTGCTCAGGACAATGTTCTTG
GTTACCCTGACACTGGTGGACAGGTTGTTTACATTCTTGATCAAGTTCGTGCTCTG
GAGATAGAGATGCTTCAACGTATTAAGCAACAAGGACTCAACATTAAACCAAGGA
TTCTCATTCTAACTCGACTTCTACCTGATGCGGTAGGAACTACATGCGGTGAACGT
CTCGAGAGAGTTTATGATTCTGAGTACTGTGATATTCTTCGTGTGCCCTTCAGAAC
AGAGAAGGGTATTGTTCGCAAATGGATCTCAAGGTTCGAAGTCTGGCCATATCTA
GAGACTTACACCGAGGATGCTGCGGTTGAGCTATCGAAAGAATTGAATGGCAAGC
CTGACCTTATCATTGGTAACTACAGTGATGGAAATCTTGTTGCTTCTTTATTGGCTC
ACAAACTTGGTGTCACTCAGTGTACCATTGCTCATGCTCTTGAGAAAACAAAGTAC
CCGGATTCTGATATCTACTGGAAGAAGCTTGACGACAAGTACCATTTCTCATGCCA
GTTCACTGCGGATATTTTCGCAATGAACCACACTGATTTCATCATCACTAGTACTTT
CCAAGAAATTGCTGGAAGCAAAGAAACTGTTGGGCAGTATGAAAGCCACACAGCC
TTTACTCTTCCCGGATTGTATCGAGTTGTTCACGGGATTGATGTGTTTGATCCCAAG
TTCAACATTGTCTCTCCTGGTGCTGATATGAGCATCTACTTCCCTTACACAGAGGA
GAAGCGTAGATTGACTAAGTTCCACTCTGAGATCGAGGAGCTCCTCTACAGCGAT
GTTGAGAACAAAGAGCACTTATGTGTGCTCAAGGACAAGAAGAAGCCGATTCTCT
TCACAATGGCTAGGCTTGATCGTGTCAAGAACTTGTCAGGTCTTGTTGAGTGGTAC
GGGAAGAACACCCGCTTGCGTGAGCTAGCTAACTTGGTTGTTGTTGGAGGAGACA
GGAGGAAAGAGTCAAAGGACAATGAAGAGAAAGCAGAGATGAAGAAAATGTATG
ATCTCATTGAGGAATACAAGCTAAACGGTCAGTTCAGGTGGATCTCCTCTCAGATG
GACCGGGTAAGGAACGGTGAGCTGTACCGGTACATCTGTGACACCAAGGGTGCTT
TTGTCCAACCTGCATTATATGAAGCCTTTGGGTTAACTGTTGTGGAGGCTATGACT
TGTGGTTTACCGACTTTCGCCACTTGCAAAGGTGGTCCAGCTGAGATCATTGTGCA
CGGTAAATCGGGTTTCCACATTGACCCTTACCATGGTGATCAGGCTGCTGATACTC
TTGCTGATTTCTTCACCAAGTGTAAGGAGGATCCATCTCACTGGGATGAGATCTCA
AAAGGAGGGCTTCAGAGGATTGAGGAGAAATACACTTGGCAAATCTATTCACAGA
GGCTCTTGACATTGACTGGTGTGTATGGATTCTGGAAGCATGTCTCGAACCTTGAC
CGTCTTGAGGCTCGCCGTTACCTTGAAATGTTCTATGCATTGAAGTATCGCCCATT
GGCTCAGGCTGTTCCTCTTGCACAAGATGATTGA
EUCP1-AtSUS1 fusion enzyme: Amino Acid Sequence (SEQ ID NO: 25)
MGSSGMSLAERFSLTLSRSSLVVGRSCVEFEPETVPLLSTLRGKPITFLGLMPPLHEGRR
EDGEDATVRWLDAQPAKSVVYVALGSEVPLGVEKVHELALGLELAGTRFLWALRKP
TGVSDADLLPAGFEERTRGRGVVATRWVPQMSILAHAAVGAFLTHCGWNSTIEGLMF
GHPLIMLPIFGDQGPNARLIEAKNAGLQVARNDGDGSFDREGVAAAIRAVAVEEESSK
VFQAKAKKLQEIVADMACHERYIDGFIQQLRSYKDDSGYSSSYAAAAGMHVVICPWL
AFGHLLPCLDLAQRLASRGHRVSFVSTPRNISRLPPVRPALAPLVAFVALPLPRVEGLP
DGAESTNDVPHDRPDMVELHRRAFDGLAAPFSEFLGTACADWVIVDVFHHWAAAAA
LEHKVPCAMMLLGSAHMIASIADRRLERAETESPAAAGQGRPAAAPTFEVARMKLIR
TKGSGANAERMITRVHSQRERLNETLVSERNEVLALLSRVEAKGKGILQQNQIIAEFEA
LPEQTRKKLEGGPFFDLLKSTQEAIVLPPWVALAVRPRPGVWEYLRVNLHALVVEEL
QPAEFLHFKEELVDGVKNGNFTLELDFEPFNASIPRPTLHKYIGNGVDFLNRHLSAKLF
HDKESLLPLLKFLRLHSHQGKNLMLSEKIQNLNTLQHTLRKAEEYLAELKSETLYEEF
EAKFEEIGLERGWGDNAERVLDMIRLLLDLLEAPDPCTLETFLGRVPMVFNVVILSPH
GYFAQDNVLGYPDTGGQVVYILDQVRALEIEMLQRIKQQGLNIKPRILILTRLLPDAVG
TTCGERLERVYDSEYCDILRVPFRTEKGIVRKWISRFEVWPYLETYTEDAAVELSKELN
GKPDLIIGNYSDGNLVASLLAHKLGVTQCTIAHALEKTKYPDSDIYWKKLDDKYHFSC
QFTADIFAMNHTDFIITSTFQEIAGSKETVGQYESHTAFTLPGLYRVVHGIDVEDPKFNI
VSPGADMSIYFPYTEEKRRLTKFHSEIEELLYSDVENKEHLCVLKDKKKPILFTMARLD
RVKNLSGLVEWYGKNTRLRELANLVVVGGDRRKESKDNEEKAEMKKMYDLIEEYK
LNGQFRWISSQMDRVRNGELYRYICDTKGAFVQPALYEAFGLTVVEAMTCGLPTFAT
CKGGPAEIIVHGKSGFHIDPYHGDQAADTLADFFTKCKEDPSHWDEISKGGLQRIEEK
YTWQIYSQRLLTLTGVYGFWKHVSNLDRLEARRYLEMFYALKYRPLAQAVPLAQDD
EUCP1-AtSUS1 fusion enzyme DNA Sequence (SEQ ID NO: 26)
ATGGGTAGCTCGGGCATGTCCCTGGCGGAACGCTTTTCGCTGACGCTGAGTCGCTC
ATCCCTGGTTGTTGGTCGCAGTTGTGTTGAATTTGAACCGGAAACCGTTCCGCTGC
TGTCTACGCTGCGCGGCAAACCGATTACCTTCCTGGGTCTGATGCCGCCGCTGCAT
GAAGGCCGTCGCGAAGATGGTGAAGACGCCACGGTGCGTTGGCTGGATGCTCAGC
CGGCGAAATCGGTGGTTTATGTCGCACTGGGCAGCGAAGTGCCGCTGGGTGTCGA
AAAAGTGCACGAACTGGCCCTGGGCCTGGAACTGGCAGGCACCCGCTTTCTGTGG
GCACTGCGTAAACCGACGGGCGTTAGCGATGCTGACCTGCTGCCGGCGGGTTTCG
AAGAACGCACCCGCGGCCGTGGTGTCGTGGCCACCCGTTGGGTGCCGCAAATGTC
CATTCTGGCTCATGCGGCCGTTGGCGCATTTCTGACCCACTGCGGTTGGAACAGCA
CGATCGAAGGCCTGATGTTTGGTCATCCGCTGATTATGCTGCCGATCTTCGGCGAT
CAGGGTCCGAACGCACGCCTGATCGAAGCCAAAAATGCAGGCCTGCAAGTTGCGC
GTAACGATGGCGACGGTAGCTTTGACCGCGAAGGTGTCGCAGCTGCGATTCGTGC
TGTGGCGGTTGAAGAAGAAAGCAGCAAAGTCTTCCAGGCCAAAGCGAAAAAACT
GCAAGAAATCGTGGCTGATATGGCGTGTCATGAACGCTATATTGACGGCTTTATCC
AGCAACTGCGTTCTTACAAAGATGACAGTGGCTATAGTTCCTCATACGCCGCAGCT
GCGGGTATGCATGTTGTCATTTGCCCGTGGCTGGCGTTTGGTCACCTGCTGCCGTG
TCTGGATCTGGCACAGCGCCTGGCATCTCGCGGTCACCGTGTTTCGTTCGTCAGCA
CCCCGCGCAATATCAGTCGTCTGCCGCCGGTTCGTCCGGCGCTGGCGCCGCTGGTT
GCGTTCGTTGCACTGCCGCTGCCGCGTGTGGAAGGTCTGCCGGATGGTGCCGAATC
GACCAACGACGTTCCGCATGATCGTCCGGACATGGTCGAACTGCATCGTCGCGCCT
TTGATGGCCTGGCCGCACCGTTTAGCGAATTTCTGGGTACGGCCTGCGCAGATTGG
GTCATTGTGGACGTTTTTCACCACTGGGCGGCGGCGGCGGCGCTGGAACATAAAG
TGCCGTGTGCGATGATGCTGCTGGGTTCCGCCCACATGATTGCTTCAATCGCGGAT
CGTCGCCTGGAACGTGCCGAAACCGAAAGTCCGGCGGCGGCAGGCCAGGGTCGTC
CGGCGGCGGCACCGACCTTTGAAGTGGCACGTATGAAACTGATTCGCACGAAAGG
TTCTGGTGCAAACGCTGAACGTATGATAACGCGCGTCCACAGCCAACGTGAGCGT
TTGAACGAAACGCTTGTTTCTGAGAGAAACGAAGTCCTTGCCTTGCTTTCCAGGGT
TGAAGCCAAAGGTAAAGGTATTTTACAACAAAACCAGATCATTGCTGAATTCGAA
GCTTTGCCTGAACAAACCCGGAAGAAACTTGAAGGTGGTCCTTTCTTTGACCTTCT
CAAATCCACTCAGGAAGCAATTGTGTTGCCACCATGGGTTGCTCTAGCTGTGAGGC
CAAGGCCTGGTGTTTGGGAATACTTACGAGTCAATCTCCATGCTCTTGTCGTTGAA
GAACTCCAACCTGCTGAGTTTCTTCATTTCAAGGAAGAACTCGTTGATGGAGTTAA
GAATGGTAATTTCACTCTTGAGCTTGATTTCGAGCCATTCAATGCGTCTATCCCTCG
TCCAACACTCCACAAATACATTGGAAATGGTGTTGACTTCCTTAACCGTCATTTAT
CGGCTAAGCTCTTCCATGACAAGGAGAGTTTGCTTCCATTGCTTAAGTTCCTTCGT
CTTCACAGCCACCAGGGCAAGAACCTGATGTTGAGCGAGAAGATTCAGAACCTCA
ACACTCTGCAACACACCTTGAGGAAAGCAGAAGAGTATCTAGCAGAGCTTAAGTC
CGAAACACTGTATGAAGAGTTTGAGGCCAAGTTTGAGGAGATTGGTCTTGAGAGG
GGATGGGGAGACAATGCAGAGCGTGTCCTTGACATGATACGTCTTCTTTTGGACCT
TCTTGAGGCGCCTGATCCTTGCACTCTTGAGACTTTTCTTGGAAGAGTACCAATGG
TGTTCAACGTTGTGATCCTCTCTCCACATGGTTACTTTGCTCAGGACAATGTTCTTG
GTTACCCTGACACTGGTGGACAGGTTGTTTACATTCTTGATCAAGTTCGTGCTCTG
GAGATAGAGATGCTTCAACGTATTAAGCAACAAGGACTCAACATTAAACCAAGGA
TTCTCATTCTAACTCGACTTCTACCTGATGCGGTAGGAACTACATGCGGTGAACGT
CTCGAGAGAGTTTATGATTCTGAGTACTGTGATATTCTTCGTGTGCCCTTCAGAAC
AGAGAAGGGTATTGTTCGCAAATGGATCTCAAGGTTCGAAGTCTGGCCATATCTA
GAGACTTACACCGAGGATGCTGCGGTTGAGCTATCGAAAGAATTGAATGGCAAGC
CTGACCTTATCATTGGTAACTACAGTGATGGAAATCTTGTTGCTTCTTTATTGGCTC
ACAAACTTGGTGTCACTCAGTGTACCATTGCTCATGCTCTTGAGAAAACAAAGTAC
CCGGATTCTGATATCTACTGGAAGAAGCTTGACGACAAGTACCATTTCTCATGCCA
GTTCACTGCGGATATTTTCGCAATGAACCACACTGATTTCATCATCACTAGTACTTT
CCAAGAAATTGCTGGAAGCAAAGAAACTGTTGGGCAGTATGAAAGCCACACAGCC
TTTACTCTTCCCGGATTGTATCGAGTTGTTCACGGGATTGATGTGTTTGATCCCAAG
TTCAACATTGTCTCTCCTGGTGCTGATATGAGCATCTACTTCCCTTACACAGAGGA
GAAGCGTAGATTGACTAAGTTCCACTCTGAGATCGAGGAGCTCCTCTACAGCGAT
GTTGAGAACAAAGAGCACTTATGTGTGCTCAAGGACAAGAAGAAGCCGATTCTCT
TCACAATGGCTAGGCTTGATCGTGTCAAGAACTTGTCAGGTCTTGTTGAGTGGTAC
GGGAAGAACACCCGCTTGCGTGAGCTAGCTAACTTGGTTGTTGTTGGAGGAGACA
GGAGGAAAGAGTCAAAGGACAATGAAGAGAAAGCAGAGATGAAGAAAATGTATG
ATCTCATTGAGGAATACAAGCTAAACGGTCAGTTCAGGTGGATCTCCTCTCAGATG
GACCGGGTAAGGAACGGTGAGCTGTACCGGTACATCTGTGACACCAAGGGTGCTT
TTGTCCAACCTGCATTATATGAAGCCTTTGGGTTAACTGTTGTGGAGGCTATGACT
TGTGGTTTACCGACTTTCGCCACTTGCAAAGGTGGTCCAGCTGAGATCATTGTGCA
CGGTAAATCGGGTTTCCACATTGACCCTTACCATGGTGATCAGGCTGCTGATACTC
TTGCTGATTTCTTCACCAAGTGTAAGGAGGATCCATCTCACTGGGATGAGATCTCA
AAAGGAGGGCTTCAGAGGATTGAGGAGAAATACACTTGGCAAATCTATTCACAGA
GGCTCTTGACATTGACTGGTGTGTATGGATTCTGGAAGCATGTCTCGAACCTTGAC
CGTCTTGAGGCTCGCCGTTACCTTGAAATGTTCTATGCATTGAAGTATCGCCCATT
GGCTCAGGCTGTTCCTCTTGCACAAGATGATTGA
HV1-AtSUS1 fusion enzyme: Amino Acid Sequence (SEQ ID NO: 27)
MDGNSSSSPLHVVICPWLALGHLLPCLDIAERLASRGHRVSFVSTPRNIARLPPLRPAV
APLVDFVALPLPHVDGLPEGAESTNDVPYDKFELHRKAFDGLAAPFSEFLRAACAEGA
GSRPDWLIVDTFHHWAAAAAVENKVPCVMLLLGAATVIAGFARGVSEHAAAAVGKE
RPAAEAPSFETERRKLMTTQNASGMTVAERYFLTLMRSDLVAIRSCAEWEPESVAALT
TLAGKPVVPLGLLPPSPEGGRGVSKEDAAVRWLDAQPAKSVVYVALGSEVPLRAEQV
HELALGLELSGARFLWALRKPTDAPDAAVLPPGFEERTRGRGLVVTGWVPQIGVLAH
GAVAAFLTHCGWNSTIEGLLFGHPLIMLPISSDQGPNARLMEGRKVGMQVPRDESDGS
FRREDVAATVRAVAVEEDGRRVFTANAKKMQEIVADGACHERCIDGFIQQLRSYKAG
SGANAERMITRVHSQRERLNETLVSERNEVLALLSRVEAKGKGILQQNQIIAEFEALPE
QTRKKLEGGPFFDLLKSTQEAIVLPPWVALAVRPRPGVWEYLRVNLHALVVEELQPA
EFLHFKEELVDGVKNGNFTLELDFEPFNASIPRPTLHKYIGNGVDFLNRHLSAKLFHDK
ESLLPLLKFLRLHSHQGKNLMLSEKIQNLNTLQHTLRKAEEYLAELKSETLYEEFEAKF
EEIGLERGWGDNAERVLDMIRLLLDLLEAPDPCTLETFLGRVPMVFNVVILSPHGYFA
QDNVLGYPDTGGQVVYILDQVRALEIEMLQRIKQQGLNIKPRILILTRLLPDAVGTTCG
ERLERVYDSEYCDILRVPFRTEKGIVRKWISRFEVWPYLETYTEDAAVELSKELNGKP
DLIIGNYSDGNLVASLLAHKLGVTQCTIAHALEKTKYPDSDIYWKKLDDKYHFSCQFT
ADIFAMNHTDFIITSTFQEIAGSKETVGQYESHTAFTLPGLYRVVHGIDVFDPKFNIVSP
GADMSIYFPYTEEKRRLTKFHSEIEELLYSDVENKEHLCVLKDKKKPILFTMARLDRV
KNLSGLVEWYGKNTRLRELANLVVVGGDRRKESKDNEEKAEMKKMYDLIEEYKLN
GQFRWISSQMDRVRNGELYRYICDTKGAFVQPALYEAFGLTVVEAMTCGLPTFATCK
GGPAEIIVHGKSGFHIDPYHGDQAADTLADFFTKCKEDPSHWDEISKGGLQRIEEKYT
WQIYSQRLLTLTGVYGFWKHVSNLDRLEARRYLEMFYALKYRPLAQAVPLAQDD
HV1-AtSUS1 fusion enzyme: DNA Sequence (SEQ ID NO: 28)
ATGGATGGTAACTCCTCCTCCTCGCCGCTGCATGTGGTCATTTGTCCGTGGCTGGC
TCTGGGTCACCTGCTGCCGTGTCTGGATATTGCTGAACGTCTGGCGTCACGCGGCC
ATCGTGTCAGTTTTGTGTCCACCCCGCGCAACATTGCCCGTCTGCCGCCGCTGCGT
CCGGCTGTTGCACCGCTGGTTGATTTCGTCGCACTGCCGCTGCCGCATGTTGACGG
TCTGCCGGAGGGTGCGGAATCGACCAATGATGTGCCGTATGACAAATTTGAACTG
CACCGTAAGGCGTTCGATGGTCTGGCGGCCCCGTTTAGCGAATTTCTGCGTGCAGC
TTGCGCAGAAGGTGCAGGTTCTCGCCCGGATTGGCTGATTGTGGACACCTTTCATC
ACTGGGCGGCGGCGGCGGCGGTGGAAAACAAAGTGCCGTGTGTTATGCTGCTGCT
GGGTGCAGCAACGGTGATCGCTGGTTTCGCGCGTGGTGTTAGCGAACATGCGGCG
GCGGCGGTGGGTAAAGAACGTCCGGCTGCGGAAGCCCCGAGTTTTGAAACCGAAC
GTCGCAAGCTGATGACCACGCAGAATGCCTCCGGCATGACCGTGGCAGAACGCTA
TTTCCTGACGCTGATGCGTAGCGATCTGGTTGCCATCCGCTCTTGCGCAGAATGGG
AACCGGAAAGCGTGGCAGCACTGACCACGCTGGCAGGTAAACCGGTGGTTCCGCT
GGGTCTGCTGCCGCCGAGTCCGGAAGGCGGTCGTGGCGTTTCCAAAGAAGATGCT
GCGGTCCGTTGGCTGGACGCACAGCCGGCAAAGTCAGTCGTGTACGTCGCACTGG
GTTCGGAAGTGCCGCTGCGTGCGGAACAAGTTCACGAACTGGCACTGGGCCTGGA
ACTGAGCGGTGCTCGCTTTCTGTGGGCGCTGCGTAAACCGACCGATGCACCGGAC
GCCGCAGTGCTGCCGCCGGGTTTCGAAGAACGTACCCGCGGCCGTGGTCTGGTTGT
CACGGGTTGGGTGCCGCAGATTGGCGTTCTGGCTCATGGTGCGGTGGCTGCGTTTC
TGACCCACTGTGGCTGGAACTCTACGATCGAAGGCCTGCTGTTCGGTCATCCGCTG
ATTATGCTGCCGATCAGCTCTGATCAGGGTCCGAATGCGCGCCTGATGGAAGGCC
GTAAAGTCGGTATGCAAGTGCCGCGTGATGAATCAGACGGCTCGTTTCGTCGCGA
AGATGTTGCCGCAACCGTCCGCGCCGTGGCAGTTGAAGAAGACGGTCGTCGCGTC
TTCACGGCTAACGCGAAAAAGATGCAAGAAATTGTGGCCGATGGCGCATGCCACG
AACGTTGTATTGACGGTTTTATCCAGCAACTGCGCAGTTACAAGGCGGGTTCTGGT
GCAAACGCTGAACGTATGATAACGCGCGTCCACAGCCAACGTGAGCGTTTGAACG
AAACGCTTGTTTCTGAGAGAAACGAAGTCCTTGCCTTGCTTTCCAGGGTTGAAGCC
AAAGGTAAAGGTATTTTACAACAAAACCAGATCATTGCTGAATTCGAAGCTTTGC
CTGAACAAACCCGGAAGAAACTTGAAGGTGGTCCTTTCTTTGACCTTCTCAAATCC
ACTCAGGAAGCAATTGTGTTGCCACCATGGGTTGCTCTAGCTGTGAGGCCAAGGC
CTGGTGTTTGGGAATACTTACGAGTCAATCTCCATGCTCTTGTCGTTGAAGAACTC
CAACCTGCTGAGTTTCTTCATTTCAAGGAAGAACTCGTTGATGGAGTTAAGAATGG
TAATTTCACTCTTGAGCTTGATTTCGAGCCATTCAATGCGTCTATCCCTCGTCCAAC
ACTCCACAAATACATTGGAAATGGTGTTGACTTCCTTAACCGTCATTTATCGGCTA
AGCTCTTCCATGACAAGGAGAGTTTGCTTCCATTGCTTAAGTTCCTTCGTCTTCACA
GCCACCAGGGCAAGAACCTGATGTTGAGCGAGAAGATTCAGAACCTCAACACTCT
GCAACACACCTTGAGGAAAGCAGAAGAGTATCTAGCAGAGCTTAAGTCCGAAACA
CTGTATGAAGAGTTTGAGGCCAAGTTTGAGGAGATTGGTCTTGAGAGGGGATGGG
GAGACAATGCAGAGCGTGTCCTTGACATGATACGTCTTCTTTTGGACCTTCTTGAG
GCGCCTGATCCTTGCACTCTTGAGACTTTTCTTGGAAGAGTACCAATGGTGTTCAA
CGTTGTGATCCTCTCTCCACATGGTTACTTTGCTCAGGACAATGTTCTTGGTTACCC
TGACACTGGTGGACAGGTTGTTTACATTCTTGATCAAGTTCGTGCTCTGGAGATAG
AGATGCTTCAACGTATTAAGCAACAAGGACTCAACATTAAACCAAGGATTCTCAT
TCTAACTCGACTTCTACCTGATGCGGTAGGAACTACATGCGGTGAACGTCTCGAGA
GAGTTTATGATTCTGAGTACTGTGATATTCTTCGTGTGCCCTTCAGAACAGAGAAG
GGTATTGTTCGCAAATGGATCTCAAGGTTCGAAGTCTGGCCATATCTAGAGACTTA
CACCGAGGATGCTGCGGTTGAGCTATCGAAAGAATTGAATGGCAAGCCTGACCTT
ATCATTGGTAACTACAGTGATGGAAATCTTGTTGCTTCTTTATTGGCTCACAAACTT
GGTGTCACTCAGTGTACCATTGCTCATGCTCTTGAGAAAACAAAGTACCCGGATTC
TGATATCTACTGGAAGAAGCTTGACGACAAGTACCATTTCTCATGCCAGTTCACTG
CGGATATTTTCGCAATGAACCACACTGATTTCATCATCACTAGTACTTTCCAAGAA
ATTGCTGGAAGCAAAGAAACTGTTGGGCAGTATGAAAGCCACACAGCCTTTACTC
TTCCCGGATTGTATCGAGTTGTTCACGGGATTGATGTGTTTGATCCCAAGTTCAAC
ATTGTCTCTCCTGGTGCTGATATGAGCATCTACTTCCCTTACACAGAGGAGAAGCG
TAGATTGACTAAGTTCCACTCTGAGATCGAGGAGCTCCTCTACAGCGATGTTGAGA
ACAAAGAGCACTTATGTGTGCTCAAGGACAAGAAGAAGCCGATTCTCTTCACAAT
GGCTAGGCTTGATCGTGTCAAGAACTTGTCAGGTCTTGTTGAGTGGTACGGGAAG
AACACCCGCTTGCGTGAGCTAGCTAACTTGGTTGTTGTTGGAGGAGACAGGAGGA
AAGAGTCAAAGGACAATGAAGAGAAAGCAGAGATGAAGAAAATGTATGATCTCA
TTGAGGAATACAAGCTAAACGGTCAGTTCAGGTGGATCTCCTCTCAGATGGACCG
GGTAAGGAACGGTGAGCTGTACCGGTACATCTGTGACACCAAGGGTGCTTTTGTC
CAACCTGCATTATATGAAGCCTTTGGGTTAACTGTTGTGGAGGCTATGACTTGTGG
TTTACCGACTTTCGCCACTTGCAAAGGTGGTCCAGCTGAGATCATTGTGCACGGTA
AATCGGGTTTCCACATTGACCCTTACCATGGTGATCAGGCTGCTGATACTCTTGCT
GATTTCTTCACCAAGTGTAAGGAGGATCCATCTCACTGGGATGAGATCTCAAAAG
GAGGGCTTCAGAGGATTGAGGAGAAATACACTTGGCAAATCTATTCACAGAGGCT
CTTGACATTGACTGGTGTGTATGGATTCTGGAAGCATGTCTCGAACCTTGACCGTC
TTGAGGCTCGCCGTTACCTTGAAATGTTCTATGCATTGAAGTATCGCCCATTGGCT
CAGGCTGTTCCTCTTGCACAAGATGATTGA
Arabidopsis thaliana sucrose synthase I: Amino Acid Sequence (SEQ ID NO: 29)
MANAERMITRVHSQRERLNETLVSERNEVLALLSRVEAKGKGILQQNQIIAEFEALPE
QTRKKLEGGPFFDLLKSTQEAIVLPPWVALAVRPRPGVWEYLRVNLHALVVEELQPA
EFLHFKEELVDGVKNGNFTLELDFEPFNASIPRPTLHKYIGNGVDFLNRHLSAKLFHDK
ESLLPLLKFLRLHSHQGKNLMLSEKIQNLNTLQHTLRKAEEYLAELKSETLYEEFEAKF
EEIGLERGWGDNAERVLDMIRLLLDLLEAPDPCTLETFLGRVPMVFNVVILSPHGYFA
QDNVLGYPDTGGQVVYILDQVRALEIEMLQRIKQQGLNIKPRILILTRLLPDAVGTTCG
ERLERVYDSEYCDILRVPFRTEKGIVRKWISRFEVWPYLETYTEDAAVELSKELNGKP
DLIIGNYSDGNLVASLLAHKLGVTQCTIAHALEKTKYPDSDIYWKKLDDKYHFSCQFT
ADIFAMNHTDFIITSTFQEIAGSKETVGQYESHTAFTLPGLYRVVHGIDVFDPKFNIVSP
GADMSIYFPYTEEKRRLTKFHSEIEELLYSDVENKEHLCVLKDKKKPILFTMARLDRV
KNLSGLVEWYGKNTRLRELANLVVVGGDRRKESKDNEEKAEMKKMYDLIEEYKLN
GQFRWISSQMDRVRNGELYRYICDTKGAFVQPALYEAFGLTVVEAMTCGLPTFATCK
GGPAEIIVHGKSGFHIDPYHGDQAADTLADFFTKCKEDPSHWDEISKGGLQRIEEKYT
WQIYSQRLLTLTGVYGFWKHVSNLDRLEARRYLEMFYALKYRPLAQAVPLAQDD
Arabidopsis thaliana sucrose synthase I: DNA Sequence (SEQ ID NO: 30)
ATGGCAAACGCTGAACGTATGATTACCCGTGTCCACTCCCAACGCGAACGCCTGA
ACGAAACCCTGGTGTCGGAACGCAACGAAGTTCTGGCACTGCTGAGCCGTGTGGA
AGCTAAGGGCAAAGGTATTCTGCAGCAAAACCAGATTATCGCGGAATTTGAAGCC
CTGCCGGAACAAACCCGCAAAAAGCTGGAAGGCGGTCCGTTTTTCGATCTGCTGA
AATCTACGCAGGAAGCGATCGTTCTGCCGCCGTGGGTCGCACTGGCAGTGCGTCC
GCGTCCGGGCGTTTGGGAATATCTGCGTGTCAACCTGCATGCACTGGTGGTTGAAG
AACTGCAGCCGGCTGAATTTCTGCACTTCAAGGAAGAACTGGTTGACGGCGTCAA
AAACGGTAATTTTACCCTGGAACTGGATTTTGAACCGTTCAATGCCAGTATCCCGC
GTCCGACGCTGCATAAATATATTGGCAACGGTGTGGACTTTCTGAATCGCCATCTG
AGCGCAAAGCTGTTCCACGATAAAGAATCTCTGCTGCCGCTGCTGAAATTCCTGCG
TCTGCATAGTCACCAGGGCAAGAACCTGATGCTGTCCGAAAAAATTCAGAACCTG
AATACCCTGCAACACACGCTGCGCAAGGCGGAAGAATACCTGGCCGAACTGAAAA
GTGAAACCCTGTACGAAGAATTCGAAGCAAAGTTCGAAGAAATTGGCCTGGAACG
TGGCTGGGGTGACAATGCTGAACGTGTTCTGGATATGATCCGTCTGCTGCTGGACC
TGCTGGAAGCACCGGACCCGTGCACCCTGGAAACGTTTCTGGGTCGCGTGCCGAT
GGTTTTCAACGTCGTGATTCTGTCCCCGCATGGCTATTTTGCACAGGACAATGTGC
TGGGTTACCCGGATACCGGCGGTCAGGTTGTCTATATTCTGGATCAAGTTCGTGCG
CTGGAAATTGAAATGCTGCAGCGCATCAAGCAGCAAGGCCTGAACATCAAACCGC
GTATTCTGATCCTGACCCGTCTGCTGCCGGATGCAGTTGGTACCACGTGCGGTGAA
CGTCTGGAACGCGTCTATGACAGCGAATACTGTGATATTCTGCGTGTCCCGTTTCG
CACCGAAAAGGGTATTGTGCGTAAATGGATCAGTCGCTTCGAAGTTTGGCCGTATC
TGGAAACCTACACGGAAGATGCGGCCGTGGAACTGTCCAAGGAACTGAATGGCAA
ACCGGACCTGATTATCGGCAACTATAGCGATGGTAATCTGGTCGCATCTCTGCTGG
CTCATAAACTGGGTGTGACCCAGTGCACGATTGCACACGCTCTGGAAAAGACCAA
ATATCCGGATTCAGACATCTACTGGAAAAAGCTGGATGACAAATATCATTTTTCGT
GTCAGTTCACCGCGGACATTTTTGCCATGAACCACACGGATTTTATTATCACCAGT
ACGTTCCAGGAAATCGCGGGCTCCAAAGAAACCGTGGGTCAATACGAATCACATA
CCGCCTTCACGCTGCCGGGCCTGTATCGTGTGGTTCACGGTATCGATGTTTTTGAC
CCGAAATTCAATATTGTCAGTCCGGGCGCGGATATGTCCATCTATTTTCCGTACAC
CGAAGAAAAGCGTCGCCTGACGAAATTCCATTCAGAAATTGAAGAACTGCTGTAC
TCGGACGTGGAAAACAAGGAACACCTGTGTGTTCTGAAAGATAAAAAGAAACCG
ATCCTGTTTACCATGGCCCGTCTGGATCGCGTGAAGAATCTGTCAGGCCTGGTTGA
ATGGTATGGTAAAAACACGCGTCTGCGCGAACTGGCAAATCTGGTCGTGGTTGGC
GGTGACCGTCGCAAGGAATCGAAAGATAACGAAGAAAAGGCTGAAATGAAGAAA
ATGTACGATCTGATCGAAGAATACAAGCTGAACGGCCAGTTTCGTTGGATCAGCT
CTCAAATGGACCGTGTGCGCAATGGCGAACTGTATCGCTACATTTGCGATACCAA
GGGTGCGTTTGTTCAGCCGGCACTGTACGAAGCTTTCGGCCTGACCGTCGTGGAAG
CCATGACGTGCGGTCTGCCGACCTTTGCGACGTGTAAAGGCGGTCCGGCCGAAAT
TATCGTGCATGGCAAATCTGGTTTCCATATCGATCCGTATCACGGTGATCAGGCAG
CTGACACCCTGGCGGATTTCTTTACGAAGTGTAAAGAAGACCCGTCACACTGGGA
TGAAATTTCGAAGGGCGGTCTGCAACGTATCGAAGAAAAATATACCTGGCAGATT
TACAGCCAACGCCTGCTGACCCTGACGGGCGTCTACGGTTTTTGGAAACATGTGTC
TAATCTGGATCGCCTGGAAGCCCGTCGCTATCTGGAAATGTTTTACGCACTGAAGT
ATCGCCCGCTGGCACAAGCCGTTCCGCTGGCACAGGACGACTAA

1. A method of producing rebaudioside R6-6, the method comprising:
(I) preparing a reaction mixture comprising:
(i) rebaudioside R5-1;
(ii) one or more substrates selected from the group comprising of sucrose, uridine diphosphate (UDP), uridine diphosphate-glucose (UDP-glucose), and combinations thereof; and
(iii) an enzyme selected from the group consisting of:
(a) a UDP-glycosyltransferase (UGT);
(b) a UDP-glycosyltransferase and a sucrose synthase; and
(c) a UDP-glycosyltransferase fusion enzyme comprising a UDP-glycosyltransferase domain coupled to a sucrose synthase domain; and
(II) incubating the reaction mixture for a sufficient time to produce rebaudioside R6-5;
wherein the rebaudioside R5-1 has the structure of: 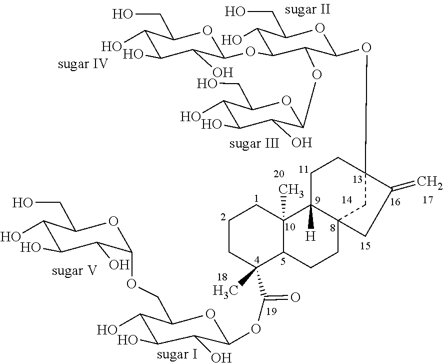
and wherein the rebaudioside R6-6 has the structure of:

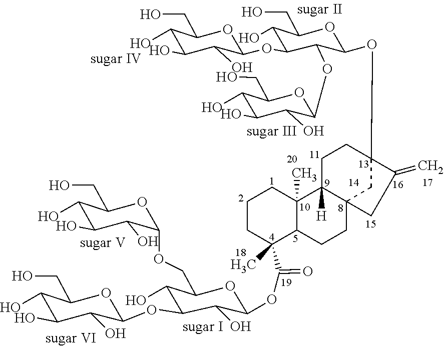

2. The method of claim 1, wherein a glucose is covalently coupled to sugar I of rebaudioside R5-1 by the enzyme to produce rebaudioside R6-6.

3. The method of claim 1, wherein the UGT is at least 80% identical to the amino acid sequence of any one of SEQ ID NOs: 9, 11, and 13.

4. The method of claim 3, wherein the UGT comprises the amino acid sequence of any one of SEQ ID NOs: 9, 11, and 13.

5. The method of claim 1, wherein the sucrose synthase or sucrose synthase domain is selected from the group consisting of an Arabidopsis sucrose synthase I, an Arabidopsis sucrose synthase 3 and a Vigna radiate sucrose synthase.

6. The method of claim 5, wherein the sucrose synthase or sucrose synthase domain is an Arabidopsis thaliana sucrose synthase I.

7. The method of claim 6, wherein the sucrose synthase or sucrose synthase domain is at least 80% identical to the amino acid sequence of SEQ ID NO: 29.

8. The method of claim 7, wherein the sucrose synthase or sucrose synthase domain comprises the amino acid sequence of SEQ ID NO: 29.

9. The method of claim 1, wherein the UDP-glycosyltransferase fusion enzyme is at least 80% identical to the amino acid sequence of any one of SEQ ID NOs: 17, 19, and 21.

10. The method of claim 9, wherein the UDP-glycosyltransferase fusion enzyme comprises the amino acid sequence of any one of SEQ ID NOs: 17, 19, and 21.

11. The method of claim 1, further comprising producing rebaudioside R5-1 by incubating rebaudioside A with a glucansucrase and a substrate selected from the group consisting of sucrose, UDP, UDP-glucose, and combinations thereof.

12. The method of claim 11, wherein a glucose is covalently coupled to sugar I of rebaudioside A by the glucansucrase to produce rebaudioside R5-1.

13. The method of claim 11, wherein the glucansucrase comprises an amino acid sequence that is at least 80% identical to SEQ ID NO: 1 or SEQ ID NO: 15.

14. The method of claim 13, wherein the glucansucrase comprises the amino acid sequence of SEQ ID NO: 1 or SEQ ID NO: 15.

15. The method of claim 1, wherein the reaction mixture is in vitro.

16. The method of claim 1, wherein the reaction mixture is a cell-based reaction mixture.

17. The method of claim 16, wherein the cell of the cell-based reaction mixture is selected from the group consisting of a yeast, a non-steviol glycoside producing plant, an alga, a fungus, and a bacterium.

18. A method of producing rebaudioside R7-5 or rebaudioside R6-5, the method comprising:
(I) preparing a reaction mixture comprising:
(i) rebaudioside R6-5 or rebaudioside R5-1;
(ii) one or more substrates selected from the group comprising of sucrose, uridine diphosphate (UDP), uridine diphosphate-glucose (UDP-glucose), and combinations thereof; and
(iii) an enzyme selected from the group consisting of:
(a) a UDP-glycosyltransferase (UGT);
(b) a UDP-glycosyltransferase and a sucrose synthase; and
(c) a UDP-glycosyltransferase fusion enzyme comprising a UDP-glycosyltransferase domain coupled to a sucrose synthase domain; and
(II) incubating the reaction mixture for a sufficient time to produce rebaudioside R7-5 or rebaudioside R6-5;
wherein the rebaudioside R5-1 has the structure of: 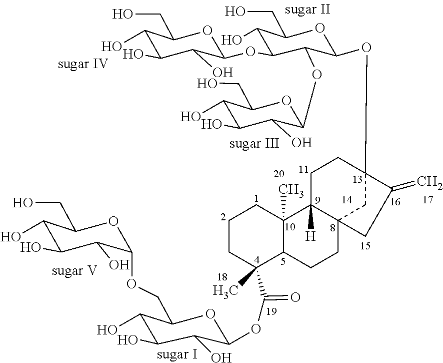
and wherein the rebaudioside R6-5 has the structure of: 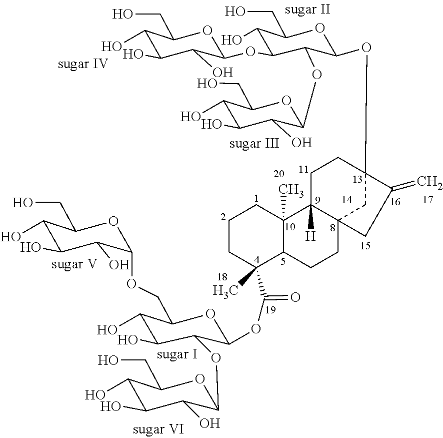
the rebaudioside R7-5 has the structure of:

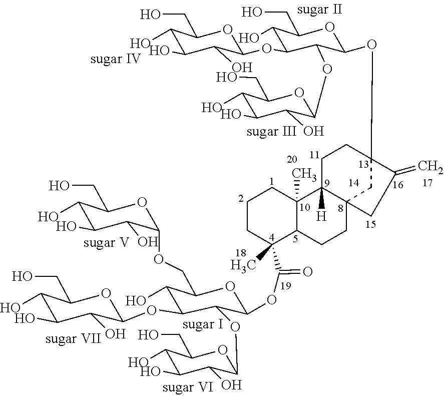

19.-55. (canceled)

56. A rebaudioside selected from:
(i) rebaudioside R6-5 having the structure: 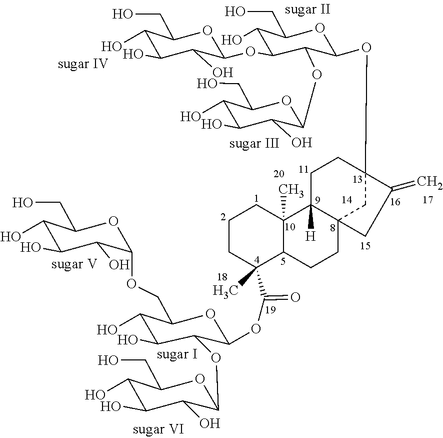
(ii) rebaudioside R6-6 having the structure: 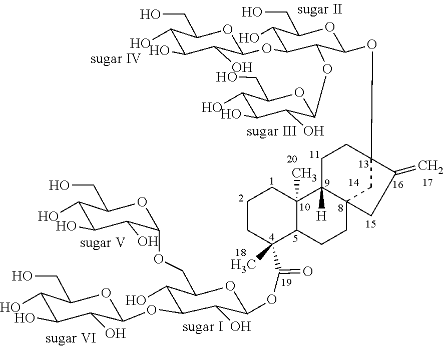
or
(iii) rebaudioside R7-5 having the structure:

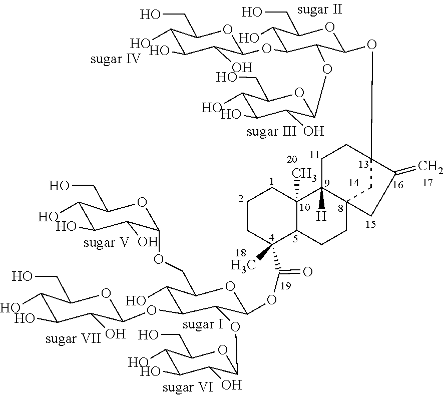

57. (canceled)

58. A composition comprising the rebaudioside of claim 56.
